(19) United States
(12) Patent Application Publication
(10) Pub. No.: US 2023/0144457 A1
(43) Pub. Date: May 11, 2023

(54) METHODS FOR TREATING VIRAL INFECTIONS, ORGAN INJURY, AND RELATED CONDITIONS

(71) Applicant: Akebia Therapeutics, Inc., Cambridge, MA (US)

(72) Inventors: Steven Burke, Cambridge, MA (US); Michael Rabinowitz, Cambridge, MA (US)

(21) Appl. No.: 17/974,000

(22) Filed: Oct. 26, 2022

Related U.S. Application Data
(60) Provisional application No. 63/272,437, filed on Oct. 27, 2021.

(51) Int. Cl. A61K 31/4418; A61P 11/00; A61P 29/00; A61P 31/14; A61K 9/00

(52) U.S. Cl. CPC A61K 31/4418; A61K 9/0053; A61P 11/00; A61P 29/00; A61P 31/14

(57) ABSTRACT

Disclosed herein are new therapeutic methods comprising administering compounds that can stabilize HIF and inhibit HIF prolyl hydroxylase (HIF-PH). In particular, methods described herein can be useful for treating/preventing a disease or condition in patients in need thereof, such as a patient having a viral infection such as a respiratory and/or pulmonary viral infection (e.g., an infection such as COVID-19 or a coronavirus infection). Methods described herein can also be useful for treating/preventing organ injury (e.g. organ injury that occurs concurrently or as a result of an infection). For example, methods described herein can be useful for treating or preventing acute lung injury, acute respiratory distress syndrome (ARDS), cardiovascular injury, injury to the liver, kidney diseases, and/or multi-organ failure.

### RELATED APPLICATIONS

This application claims benefit to U.S. Provisional Pat. Application No. 63/272,437, filed Oct. 27, 2021, the disclosure of which is hereby incorporated by reference in its entirety.

### BACKGROUND OF THE INVENTION

Hypoxia is a condition or state in which the supply of oxygen is insufficient for normal life function, for example, where there is low arterial oxygen supply. Hypoxia can lead to functional impairment of cells and tissue damage. The activation of cellular defense mechanisms during hypoxia is mediated by HIF (Hypoxia-inducible factor) protein. In fact, many studies demonstrate that stabilization of HIF can dampen tissue inflammation and promote its repair (Lee et al. (2019) Exp. Mol. Med. 51:68).

Coronaviruses such as the human CoV isolates 229E and OC43 cause mild and self-limiting infections of the respiratory tract such as the common cold. Novel isolates HCoV-NL63 and HCoV-HKU1 have also been associated with the common cold. Some novel emerging coronaviruses have resulted in serious global outbreaks such as Severe Acute Respiratory Syndrome (SARS) in 2003, Middle East Respiratory Syndrome (MERS) in 2012 and Coronavirus Disease 2019 (COVID-19). There is a need for new therapeutic methods for treating viral infections caused by novel coronaviruses and related conditions or in treating conditions in patients having such viral infections.

Acute lung injury (ALI) and acute respiratory distress syndrome (ARDS) are examples of clinical syndromes of acute respiratory failure with substantial morbidity and mortality. ALI and ARDS are seen in many critically ill patients infected with coronavirus. There is a need for new therapeutic methods to improve clinical outcomes and reduce mortality and morbidity from ALI and ARDS.

Described herein are new therapeutic methods comprising administering compounds that can stabilize HIF and inhibit HIF prolyl hydroxylase (HIF-PH). In particular, methods described herein can be useful for treating/preventing a disease or condition in patients in need thereof, such as a patient having a viral infection such as a respiratory and/or pulmonary viral infection (e.g., an infection such as COVID-19 or a coronavirus infection). Methods described herein can also be useful for treating/preventing organ injury (e.g. organ injury that occurs concurrently or as a result of an infection). For example, methods described herein can be useful for treating or preventing acute lung injury, acute respiratory distress syndrome (ARDS), cardiovascular injury, injury to the liver, kidney diseases and/or multi-organ failure. Exemplary embodiments are described herein.

### SUMMARY OF THE INVENTION

Described herein are therapeutic methods comprising administering to a patient in need thereof a compound that is a HIF prolyl hydroxylase inhibitor or a HIF-alpha stabilizer, or a pharmaceutically acceptable salt thereof. For example, methods described herein can be beneficial for the prevention and/or treatment of Acute Respiratory Distress Syndrome (ARDS), including ARDS in hospitalized patients with coronavirus disease 2019 (COVID-19).

In one aspect, the invention features a method of treating a respiratory viral infection, comprising administering to a patient in need thereof an oral, once daily dose of vadadustat, or a pharmaceutically acceptable salt thereof.

In embodiments, a respiratory viral infection is induced by or associated with a virus that could lead to viral pneumonia.

In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus.

In embodiments, a respiratory viral infection is induced by or associated with a coronavirus.

In embodiments, a coronavirus is the SARS-CoV, MERS-CoV, or SARS-CoV-2.

In embodiments, a respiratory viral infection is lower respiratory infection.

In embodiments, a respiratory viral infection is pulmonary viral infection.

In embodiments, a respiratory viral infection is upper respiratory infection.

In embodiments, a respiratory viral infection is COVID-19.

In embodiments, a respiratory viral infection induces a lung disease.

In embodiments, a lung disease is selected from acute lung injury (ALI), bronchitis, pneumonia, pulmonary fibrosis, asthma, or acute respiratory distress syndrome (ARDS).

In embodiments, a patient develops pulmonary hypertension.

In embodiments, a patient develops multi-organ failure.

In embodiments, multi-organ failure comprises heart failure, liver failure, lung failure, kidney failure, and gastrointestinal (GI) system failure.

In another aspect, the invention features a method of treating a coronavirus infection, comprising administering to a patient in need thereof an oral, once daily dose of vadadustat, or a pharmaceutically acceptable salt thereof.

In another aspect, the invention features a method of treating or preventing a lung disease that is acute lung injury (ALI) in a patient in need thereof an oral, once daily dose of vadadustat, or a pharmaceutically acceptable salt thereof.

In embodiments, a patient has a viral infection, sepsis, pneumonia, aspiration, trauma, pancreatitis, blood transfusion, and/or smoke or toxic gas inhalation.

In embodiments, an acute lung injury (ALI) is induced by or associated with a viral infection, sepsis, pneumonia, aspiration, trauma, pancreatitis, blood transfusion, and/or smoke or toxic gas inhalation.

In embodiments, a patient is mechanically ventilated.

In another aspect, the invention features a method of treating or preventing a lung disease that is acute lung injury (ALI) in a patient having a respiratory viral infection, comprising administering to the patient an oral, once daily dose of vadadustat, or a pharmaceutically acceptable salt thereof.

In another aspect, the invention features a method of treating or preventing a lung disease that is acute respiratory distress syndrome (ARDS) in a patient having a respiratory viral infection, comprising administering to the patient an oral, once daily dose of vadadustat, or a pharmaceutically acceptable salt thereof.

In embodiments, a respiratory viral infection is a pulmonary infection. In embodiments, a respiratory viral infection is infected by a coronavirus, an influenza A, an influenza B, a Rhinovirus, a respiratory syncytial virus (RSV), an adenovirus, a human parainfluenza virus (HPIV) or an enterovirus.

In embodiments, a respiratory viral infection is infected by a coronavirus.

In embodiments, a coronavirus is the SARS-CoV, MERS-CoV, or SARS-CoV-2.

In embodiments, a respiratory viral infection is COVID-19.

In embodiments, an acute lung injury develops acute respiratory distress syndrome (ARDS).

In embodiments, a patient with ALI or ARDS develops pulmonary hypertension.

In embodiments, a patient with ALI or ARDS develops multi-organ failure.

In embodiments, multi-organ failure comprise heart failure, liver failure, lung failure kidney failure, and gastrointestinal (GI) system failure.

In embodiments, the ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

In embodiments, the ALI or ARDS is induced by or associated with the viral infection.

In another aspect, the invention features a method of treating or preventing a lung disease that is acute lung injury (ALI) in a patient infected with a coronavirus, comprising administering to the patient an oral, once daily dose of vadadustat, or a pharmaceutically acceptable salt thereof.

In another aspect, the invention features a method of treating or preventing a lung disease that is acute respiratory distress syndrome (ARDS) in a patient infected with a coronavirus, comprising administering to the patient an oral, once daily dose of vadadustat, or a pharmaceutically acceptable salt thereof.

In embodiments, a coronavirus is the SARS-CoV, MERS-CoV, or SARS-CoV-2.

In embodiments, a patient is infected with SARS-CoV-2.

In embodiments, a patient with ALI or ARDS develops pulmonary hypertension.

In embodiments, a patient with ALI or ARDS develops multi-organ failure.

In embodiments, multi-organ failure comprises heart failure, liver failure, lung failure kidney failure, and gastrointestinal (GI) system failure.

In embodiments, the ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

In embodiments, the ALI or ARDS is induced by or associated with the viral infection.

In another aspect, the invention features a method of reducing mortality and morbidity related to acute lung injury (ALI) or acute respiratory distress syndrome (ARDS) or reducing incidence, severity, or risk of acute lung injury (ALI) or acute respiratory distress syndrome (ARDS) in a patient having a respiratory viral infection, said method comprising administering to a patient in need thereof an oral, once daily dose of vadadustat, or a pharmaceutically acceptable salt thereof, optionally wherein the respiratory viral infection is a pulmonary viral infection.

In embodiments, a viral infection is induced by or associated with a virus that could lead to viral pneumonia.

In embodiments, a viral infection is induced by or associated with a coronavirus, an influenza A, an influenza B, a Rhinovirus, a respiratory syncytial virus (RSV), an adenovirus, a human parainfluenza virus (HPIV) or an enterovirus.

In another aspect, the invention features a method of reducing mortality and morbidity related to acute lung injury (ALI) or acute respiratory distress syndrome (ARDS) in a patient infected with a coronavirus, or reducing incidence, severity, or risk of acute lung injury (ALI) or acute respiratory distress syndrome (ARDS), said method comprising administering to a patient in need thereof an oral, once daily dose of vadadustat, or a pharmaceutically acceptable salt thereof.

In embodiments, an acute lung injury (ALI) or acute respiratory distress syndrome (ARDS) is induced by or associated with the virus.

In embodiments, a patient is infected by a coronavirus that is SARS-CoV, MERS-CoV, or SARS-CoV-2.

In embodiments, a patient has COVID-19.

In embodiments, a patient is receiving mechanical ventilation.

In embodiments, a patient has ventilator induced ALI.

In another aspect, the invention features a method of treating or preventing lung inflammation or pneumonia, reducing mortality and morbidity related to lung inflammation or pneumonia, or reducing incidence, severity, or risk of lung inflammation or pneumonia in a patient having a respiratory viral infection,
said method comprising administering to a patient in need thereof an oral, once daily dose of vadadustat, or a pharmaceutically acceptable salt thereof, and
optionally wherein the respiratory viral infection is a pulmonary viral infection.

In embodiments, a patient has a lower respiratory infection.

In embodiments, a patient has an upper respiratory infection.

In embodiments, a viral infection is induced by or associated with a virus that could lead to viral pneumonia.

In embodiments, a viral infection is induced by or associated with a coronavirus, an influenza A, an influenza B, a Rhinovirus, a respiratory syncytial virus (RSV), an adenovirus, a human parainfluenza virus (HPIV) or an enterovirus.

In another aspect, the invention features a method of treating or preventing lung inflammation or pneumonia reducing mortality and morbidity related to lung inflammation or pneumonia, or reducing incidence, severity, or risk of lung inflammation or pneumonia in a patient infected with a coronavirus, said method comprising administering to a patient in need thereof an oral, once daily dose of vadadustat, or a pharmaceutically acceptable salt thereof.

In embodiments, a patient is infected by a coronavirus that is SARS-CoV, MERS-CoV, or SARS-CoV-2.

In embodiments, a patient has COVID-19.

In another aspect, the invention features a method of treating or preventing COVID-19 related pneumonia, comprising administering to a patient in need thereof an oral, once daily dose of vadadustat, or a pharmaceutically acceptable salt thereof, and wherein the patient has acute lung injury(ALI), acute respiratory distress syndrome (ARDS), and/or organ failure.

In embodiments, any of the acute lung injury (ALI), acute respiratory distress syndrome (ARDS), and/or organ failure is associated with COVID-19.

In another aspect, the invention features a method of treating or preventing COVID-19 related acute lung injury (ALI) or COVID-19 related acute respiratory distress syndrome (ARDS) in a patient having pneumonia, comprising administering to a patient in need thereof an oral, once daily dose of vadadustat, or a pharmaceutically acceptable salt thereof.

In embodiments, a patient is receiving mechanical ventilation.

In another aspect, the invention features a method of treating or preventing cardiac dysfunction, reducing mortality and morbidity related to an adverse cardiac event, or reducing incidence, severity, or risk of an adverse cardiac event in a patient having a respiratory viral infection, said method comprising administering to a patient in need thereof an oral, once daily dose of vadadustat, or a pharmaceutically acceptable salt thereof, optionally wherein the respiratory viral infection is a pulmonary viral infection.

In another aspect, the invention features a method of treating or preventing hypotension in a patient having a respiratory viral infection, reducing mortality and morbidity related to hypotension, or reducing incidence, severity, or risk of hypotension, said method comprising administering to a patient in need thereof an oral, once daily dose of vadadustat, or a pharmaceutically acceptable salt thereof, optionally wherein the respiratory viral infection is a pulmonary viral infection.

In another aspect, the invention features a method of reducing incidence, severity, or risk of disseminated intravascular coagulation, treating or preventing disseminated intravascular coagulation, or reducing mortality and morbidity related to disseminated intravascular coagulation in a patient having a respiratory viral infection, said method comprising administering to a patient in need thereof an oral, once daily dose of vadadustat, or a pharmaceutically acceptable salt thereof.

In another aspect, the invention features a method of treating or preventing kidney diseases, reducing mortality and morbidity related to kidney diseases, or reducing incidence, severity, or risk of kidney diseases in a patient having a respiratory viral infection, said method comprising administering to a patient in need thereof an oral, once daily dose of vadadustat, or a pharmaceutically acceptable salt thereof, optionally wherein the respiratory viral infection is a pulmonary viral infection.

In another aspect, the invention features a method of treating or preventing liver failure, reducing mortality and morbidity related to liver failure, or reducing incidence, severity, or risk of liver failure in a patient having a respiratory viral infection, said method comprising administering to a patient in need thereof an oral, once daily dose of vadadustat, or a pharmaceutically acceptable salt thereof, optionally wherein said respiratory viral infection is a pulmonary viral infection.

In another aspect, the invention features a method of treating or preventing pancreas injury, reducing mortality and morbidity related to pancreas injury, or reducing incidence, severity, or risk of pancreas injury in a patient having a respiratory viral infection, said method comprising administering to a patient in need thereof an oral, once daily dose of vadadustat, or a pharmaceutically acceptable salt thereof, optionally wherein said respiratory viral infection is a pulmonary viral infection.

In another aspect, the invention features a method of treating or preventing multi-organ failure, reducing mortality and morbidity related to multi-organ failure, or reducing incidence, severity, or risk of multi-organ failure in a patient having a respiratory viral infection, in a patient having a respiratory viral infection, said method comprising administering to a patient in need thereof an oral, once daily dose of vadadustat, or a pharmaceutically acceptable salt thereof, optionally wherein the respiratory viral infection is a pulmonary viral infection.

In another aspect, the invention features a method of treating or preventing septic shock, reducing mortality and morbidity related to sepsis, or reducing incidence, severity, or risk of sepsis in a patient having a respiratory viral infection in a patient having a respiratory viral infection, said method comprising administering to a patient in need thereof an oral, once daily dose of vadadustat, or a pharmaceutically acceptable salt thereof, optionally wherein said respiratory viral infection is a pulmonary viral infection.

In another aspect, the invention features a method of treating or preventing cytokine release syndrome, reducing mortality and morbidity related to cytokine release syndrome, or reducing incidence, severity, or risk of cytokine release syndrome in a patient having a respiratory viral infection, said method comprising administering to a patient in need thereof an oral, once daily dose of vadadustat, or a pharmaceutically acceptable salt thereof, optionally wherein the respiratory viral infection is a pulmonary viral infection.

In another aspect, the invention features a method of treating or preventing neurological disorder, reducing mortality and morbidity related to neurological disorder, or reducing incidence, severity, or risk of neurological disorder in a patient having a respiratory viral infection, said method comprising administering to a patient in need thereof an oral, once daily dose of vadadustat, or a pharmaceutically acceptable salt thereof, optionally wherein said respiratory viral infection is a pulmonary viral infection.

In another aspect, the invention features a method of treating or preventing pulmonary barotrauma, reducing mortality and morbidity related to pulmonary barotrauma, or reducing incidence, severity, or risk related to pulmonary barotrauma in a patient having a respiratory viral infection, said method comprising administering to a patient in need thereof an oral, once daily dose of vadadustat, or a pharmaceutically acceptable salt thereof, optionally wherein said respiratory viral infection is a pulmonary viral infection.

In embodiments, a patient has a lower respiratory infection.

In embodiments, a patient has an upper respiratory infection.

In embodiments, a viral infection is induced by or associated with a virus that could lead to viral pneumonia.

In embodiments, a viral infection is induced by or associated with a coronavirus, an influenza A, an influenza B, a Rhinovirus, a respiratory syncytial virus (RSV), an adenovirus, a human parainfluenza virus (HPIV) or an enterovirus.

In another aspect, the invention features a method of treating or preventing cardiac dysfunction, reducing mortality and morbidity related to an adverse cardiac event, or reducing incidence, severity, or risk of an adverse cardiac event in a patient infected with a coronavirus, said method comprising administering to a patient in need thereof an oral, once daily dose of vadadustat, or a pharmaceutically acceptable salt thereof.

In another aspect, the invention features a method of treating or preventing hypotension, reducing mortality and morbidity related to hypotension, or reducing incidence, severity, or risk of hypotension in a patient infected with a coronavirus, said method comprising administering to a patient in need thereof an oral, once daily dose of vadadustat, or a pharmaceutically acceptable salt thereof.

In another aspect, the invention features a method of treating or preventing disseminated intravascular coagulation, reducing mortality and morbidity related to disseminated intravascular coagulation, or reducing incidence, severity, or risk of disseminated intravascular coagulation in a patient infected with a coronavirus, said method comprising administering to a patient in need thereof an oral, once daily dose of vadadustat, or a pharmaceutically acceptable salt thereof.

In another aspect, the invention features a method of treating or preventing kidney diseases, reducing mortality and morbidity related to kidney diseases, or reducing incidence, severity, or risk of kidney diseases in a patient infected with a coronavirus, said method comprising administering to a patient in need thereof an oral, once daily dose of vadadustat, or a pharmaceutically acceptable salt thereof.

In another aspect, the invention features a method of treating or preventing liver failure, reducing mortality and morbidity related to liver failure, or reducing incidence, severity, or risk of liver failure in a patient infected with a coronavirus, said method comprising administering to a patient in need thereof an oral, once daily dose of vadadustat, or a pharmaceutically acceptable salt thereof.

In another aspect, the invention features a method of treating or preventing pancreas injury, reducing mortality and morbidity related to pancreas injury, or reducing incidence, severity, or risk of pancreas injury in a patient infected with a coronavirus, said method comprising administering to a patient in need thereof an oral, once daily dose of vadadustat, or a pharmaceutically acceptable salt thereof.

In another aspect, the invention features a method of treating or preventing multi-organ failure, reducing mortality and morbidity related to multi-organ failure, or reducing incidence, severity, or risk of multi-organ failure in a patient having a coronavirus infection, said method comprising administering to a patient in need thereof an oral, once daily dose of vadadustat, or a pharmaceutically acceptable salt thereof.

In another aspect, the invention features a method of treating or preventing septic shock, reducing mortality and morbidity related to septic shock, or reducing incidence, severity, or risk of septic shock in a patient infected with a coronavirus, said method comprising administering to a patient in need thereof an oral, once daily dose of vadadustat, or a pharmaceutically acceptable salt thereof.

In another aspect, the invention features a method of treating or preventing sepsis, reducing mortality and morbidity related to sepsis, or reducing incidence, severity, or risk of sepsis in a patient infected with a coronavirus, said method comprising administering to a patient in need thereof an oral, once daily dose of vadadustat, or a pharmaceutically acceptable salt thereof.

In another aspect, the invention features a method of treating or preventing cytokine release syndrome, reducing mortality and morbidity related to cytokine release syndrome, or reducing incidence, severity, or risk of cytokine release syndrome in a patient infected with a coronavirus, said method comprising administering to a patient in need thereof an oral, once daily dose of vadadustat, or a pharmaceutically acceptable salt thereof.

In another aspect, the invention features a method of treating or preventing neurological disorder, reducing mortality and morbidity related to neurological disorder, or reducing incidence, severity, or risk of neurological disorder in a patient infected with a coronavirus, said method comprising administering to a patient in need thereof an oral, once daily dose of vadadustat, or a pharmaceutically acceptable salt thereof.

In another aspect, the invention features a method of treating or preventing pulmonary barotrauma, reducing mortality and morbidity related to pulmonary barotrauma, or reducing incidence, severity, or risk of pulmonary barotrauma in a patient infected with a coronavirus, said method comprising administering to a patient in need thereof an oral, once daily dose of vadadustat, or a pharmaceutically acceptable salt thereof.

In embodiments, a patient is infected by a coronavirus that is SARS-CoV, MERS-CoV, or SARS-CoV-2.

In embodiments, a patient has COVID-19.

In embodiments, the cardiac dysfunction is induced by or associated with the viral infection, optionally wherein the cardiac dysfunction is induced by or associated with COVID-19.

In embodiments, an adverse cardiac event is induced by or associated with the viral infection, optionally wherein an adverse cardiac event is induced by or associated with COVID-19.

In embodiments, the hypotension is induced by or associated with the viral infection, optionally wherein the hypotension is induced by or associated with COVID-19.

In embodiments, the disseminated intravascular coagulation is induced by or associated with the viral infection, optionally wherein the disseminated intravascular coagulation is induced by or associated with COVID-19.

In embodiments, the kidney diseases is induced by or associated with the viral infection, optionally wherein the kidney diseases is induced by or associated with COVID-19.

In embodiments, the liver failure is induced by or associated with the viral infection, optionally wherein the liver failure is induced by or associated with COVID-19.

In embodiments, the pancreas injury is induced by or associated with the viral infection, optionally wherein the pancreas injury is induced by or associated with COVID-19.

In embodiments, the multi-organ failure is induced by or associated with the viral infection, optionally wherein the multi-organ failure is induced by or associated with COVID-19.

In embodiments, the septic shock is induced by or associated with the viral infection, optionally wherein the septic shock is induced by or associated with COVID-19.

In embodiments, the sepsis is induced by or associated with the viral infection, optionally wherein the sepsis is induced by or associated with COVID-19.

In embodiments, the cytokine release syndrome is induced by or associated with the viral infection, optionally wherein the cytokine release syndrome is induced by or associated with COVID-19.

In embodiments, the neurological disorder is induced by or associated with the viral infection, optionally wherein the neurological disorder is induced by or associated with COVID-19.

In embodiments, the pulmonary barotrauma is induced by or associated with the viral infection, optionally wherein the pulmonary barotrauma is induced by or associated with COVID-19.

In embodiments, the patient is receiving mechanical ventilation.

In another aspect, the invention feafures a method of treating or preventing organ dysfunction induced by or associated with acute lung injury (ALI), acute respiratory distress syndrome (ARDS), pneumonia, lung inflammation, or any combination thereof, comprising administering to a patient in need thereof an oral, once daily dose of vadadustat, or a pharmaceutically acceptable salt thereof.

In embodiments, the patient is infected with coronavirus.

In embodiments, the patient is infected by a coronavirus that is SARS-CoV, MERS-CoV, or SARS-CoV-2.

In embodiments, the patient has COVID-19.

In embodiments, the patient has a respiratory viral infection, optionally wherein the respiratory viral infection is a pulmonary infection.

In embodiments, the patient has a lower respiratory infection.

In embodiments, the patient has an upper respiratory infection.

In another aspect, the invention feafures a method for improving lung volumes in a patient in need thereof, comprising administering to a patient in need thereof an oral, once daily dose of vadadustat, or a pharmaceutically acceptable salt thereof.

In another aspect, the invention feafures a method for improving pulmonary compliance in a patient in need thereof, comprising administering to a patient in need thereof an oral, once daily dose of vadadustat, or a pharmaceutically acceptable salt thereof.

In another aspect, the invention feafures a method for promoting fibroproliferative repair in a patient in need thereof, comprising administering to a patient in need thereof an oral, once daily dose of vadadustat, or a pharmaceutically acceptable salt thereof.

In another aspect, the invention feafures a method for regulating innate and adaptive immunity, comprising administering to a patient in need thereof an oral, once daily dose of vadadustat, or a pharmaceutically acceptable salt thereof.

In another aspect, the invention feafures a method for promoting regulatory T-cells, lymphocytes and B cells, comprising administering to a patient in need thereof an oral, once daily dose of vadadustat, or a pharmaceutically acceptable salt thereof.

In another aspect, the invention feafures a method for treating/preventing bronchial inflammation, comprising administering to a patient in need thereof an oral, once daily dose of vadadustat, or a pharmaceutically acceptable salt thereof.

In another aspect, the invention feafures a method for reducing cytokine production, comprising administering to a patient in need thereof an oral, once daily dose of vadadustat, or a pharmaceutically acceptable salt thereof.

In another aspect, the invention feafures a method for improving dyspnea, comprising administering to a patient in need thereof an oral, once daily dose of vadadustat, or a pharmaceutically acceptable salt thereof.

In another aspect, the invention feafures a method for improving arterial PaO₂, comprising administering to a patient in need thereof an oral, once daily dose of vadadustat, or a pharmaceutically acceptable salt thereof.

In embodiments, the patient has a viral infection, optionally wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

In embodiments, the viral infection is induced by or associated with coronavirus, optionally wherein the coronavirus is the SARS-CoV, MERS-CoV, or SARS-CoV-2.

In embodiments, the patient has COVID-19.

In embodiments, said patient is an adult.

In embodiments, said patient exhibits one or more symptoms of hypoxia, optionally wherein the one or more symptoms of hypoxia include changes in the color of the skin, confusion, cough, fast heart rate, rapid breathing, shortness of breath, sweating, and/or wheezing; and/or said patient has a low oxygen saturation of hemoglobin, optionally an oxygen saturation of hemoglobin that is about 94% or less.

In another aspect, the invention feafures a method of treating or preventing acute respiratory distress syndrome (ARDS) in a hospitalized patient with COVID-19, the method comprising administering to said patient an oral, once daily dose of vadadustat or a pharmaceutically acceptable salt thereof.

In embodiments, said patient is an adult.

In embodiments, said patient exhibits one or more symptoms of hypoxia.

In embodiments, the patient has a lower oxygen saturation of hemoglobin compared to normal oxygen saturation.

In embodiments, the patient has an oxygen saturation of hemoglobin that is about 94% or less.

In embodiments, the patient has a pre-existing respiratory or pulmonary disease.

In embodiments, the pre-existing respiratory or pulmonary disease is acute lung injury, bronchitis, pneumonia, pulmonary fibrosis, asthma, and acute respiratory distress syndrome, or pulmonary hypertension.

In embodiments, the patient does not have a pre-existing respiratory or pulmonary disease.

In embodiments, the patient is an adult aged 45 years and above, aged 65 years and above, aged 80 years and above, over 80 years of age, aged 45-60 years, aged 60-80 years, or has underlying medical conditions.

In embodiments, the underlying medical conditions comprise liver disease, heart conditions, kidney disease, obesity, diabetes, or being immunocompromised, optionally wherein the patient has a body mass index (BMI) that is 25 or above or is 30 or above.

In embodiments, the patient has one or more clinically-recognized symptoms associated with the respiratory viral infection or the coronavirus infection, wherein the respiratory viral infection or the coronavirus infection is COVID-19, optionally wherein the one or more symptoms associated with COVID-19 are fever, dry cough, fatigue, coughing up sputum from the lungs, bone or joint pain, sore throat, headache, chills, nausea or vomiting, stuffy nose, pressure or pain in the chest, shortness of breath, sudden confusion, digestive issues, conjunctivitis, bluish face or lips, or loss of smell or taste.

In embodiments, the patient has decreased oxygen saturation relative to normal levels, optionally wherein the decreased oxygen saturation is an oxygen saturation of about 94% or less.

In embodiments, the patient has an elevated level of an inflammation marker.

In embodiments, the elevated level of an inflammation marker is detected in the serum of the patient.

In embodiments, the elevated level of an inflammation marker is detected in the lung of the patient.

In embodiments, the inflammation marker is a presence of ground-glass opacity.

In embodiments, the patient has pulmonary inflammation.

In embodiments, the pulmonary inflammation is characterized by body weight change, cell differentials, total nucleated cell counts, BAL (bronchoalveolar lavage) cytokine concentrations, peribronchiolar lymphoid inflammation width, lung histopathology scores, or lung summed inflammation scores.

In embodiments, the body weight change is body weight loss.

In embodiments, the patient has changes in cell differentials following commencement of administering vadadustat to the patient.

In embodiments, the patient has a decreased total nucleated cell counts following commencement of administering vadadustat to the patient.

In embodiments, the patient has a decreased BAL (bronchoalveolar lavage) cytokine concentrations following commencement of administering vadadustat to the patient.

In embodiments, the cytokine is IL-6 (Interleukin-6) or TNFα (Tumor Necrosis Factor-α).

In embodiments, the lung histopathology score is characterized by neutrophil, interstitial inflammation score, peribronchiolar/perivascular edema, and/or airway epithelial damage.

In embodiments, the patient has an improvement in lung histopathology score following commencement of administering vadadustat to the patient.

In embodiments, the patient has reduced neutrophil and/or interstitial inflammation scores.

In embodiments, the patient has a reduced lung summed inflammation score following commencement of administering vadadustat to the patient.

In embodiments, the patient has a positive diagnostic test for SARS-CoV-2.

In embodiments, vadadustat (AKB-6548) is administered at a dose of 150 mg to 1000 mg per day.

In embodiments, vadadustat (AKB-6548) is administered at a dose of 150 mg to 1800 mg per day.

In embodiments, vadadustat (AKB-6548) is administered at a dose of 900 mg to 1800 mg per day.

In embodiments, vadadustat (AKB-6548) is administered at a dose of 900, 1050, 1200, 1350, 1500, 1650, or 1800 mg per day.

In embodiments, vadadustat (AKB-6548) is administered at an initial dose of 900 mg per day.

In embodiments, vadadustat (AKB-6548) is administered at a dose of 900 mg per day throughout the treatment period.

In embodiments, vadadustat is administered to the patient for up to about one month.

In embodiments, vadadustat is administered to the patient for up to about four weeks (about 28 days), three weeks (about 21 days), two weeks (about 14 days), or one week (about 7 days).

In embodiments, vadadustat is administered in combination with another treatment.

In embodiments, treatment comprises an antiviral agent or oxygen therapy.

In embodiments, oxygen therapy comprises mechanical ventilation.

In embodiments, the severity of disease is maintained or reduced following commencement of administering vadadustat to the patient.

In embodiments, the severity of disease is assessed according to the following ordinal scale where increasing numbers denote increased severity:

| Score | Descriptor | Patient State | |
|---|---|---|---|
| 0 | No clinical or virological evidence of infection | Uninfected | Uninfected/ Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | Infected -Hospitalized (Severe Disease) | |
| 6 | Patient is hospitalized and receives oxygen therapy that is non-invasive ventilation or high flow oxygen devices | | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |

In embodiments, the severity of disease is maintained and the patient’s condition stabilizes following commencement of administering vadadustat to the patient.

In embodiments, a patient’s score does not change and the patient’s condition stabilizes following commencement of administering vadadustat to the patient.

In embodiments, the severity of disease decreases following commencement of administering vadadustat to the patient.

In embodiments, a patient’s score decreases by at least one point following commencement of administering vadadustat to the patient.

In embodiments, a patient’s severity of disease is assessed as uninfected/ambulatory.

In embodiments, a patient’s severity of disease is assessed as hospitalized but not intubated.

In embodiments, a patient’s severity of disease is assessed as hospitalized and intubated.

In embodiments, a patient’s category decreases by at least one category following commencement of administering the vadadustat to the patient.

In embodiments, a patient is hypoxic.

In embodiments, a patient has an oxygen saturation value that is about 94% or less.

In embodiments, a patient’s oxygen saturation value improves to a value of about 95% or greater following commencement of administering vadadustat to the patient.

In embodiments, a patient has hypotension.

In embodiments, a patient receives vasopressor therapy.

In embodiments, a vasopressor therapy is reduced or discontinued following commencement of administering vadadustat to the patient.

In embodiments, a patient is hospitalized.

In embodiments, a patient does not receive oxygen therapy.

In embodiments, a patient has a score of 1, 2, and/or 3 according to the ordinal scale described herein.

In embodiments, a patient receives oxygen therapy.

In embodiments, a patient has a score of 4, 5, 6, or 7 according to the ordinal scale described herein.

In embodiments, a patient receives oxygen therapy by mask or nasal prongs.

In embodiments, a patient receives oxygen therapy by intubation and mechanical ventilation.

In embodiments, a patient has multi-organ injuries and receives additional organ support therapy.

In embodiments, a patient has any of a cardiovascular injury, a neurological injury, a kidney disease, a liver injury, or a pancreas injury.

In embodiments, a patient has a heart injury and/or a lung injury.

In embodiments, a patient receives organ support therapy that is vapopressor therapy, renal replacement therapy (RRT), and/or extracorporeal membrane oxygenation (ECMO).

In embodiments, an organ support therapy received by the patient is discontinued following commencement of administering vadadustat to the patient.

In embodiments, an oxygen therapy received by the patient is discontinued following commencement of administering vadadustat to the patient.

In embodiments, a patient has increased ventilator-free survival following discontinuation of mechanical ventilation.

In embodiments, a patient receives vadadustat, or a pharmaceutically acceptable salt thereof.

### BRIEF DESCRIPTION OF THE DRAWINGS

FIG. 1 compares the mean body weight change from study day -2 for mice receiving different treatments. Significantly increased body weight loss was observed at 6 hour post-LPS in mice treated with 300 mg/kg Compound 1 as compared to the vehicle disease control group.

FIG. 2 compares the mean cell type of total cells per milliliter in BAL fluids for mice receiving different treatments. Significant changes in cell differentials were observed at 24 hours post-LPS in mice treated with 100 or 300 mg/kg Compound 1 as compared to the vehicle disease control group.

FIG. 3 compares the mean BAL total nucleated cells for mice receiving different treatments. Significantly reduced total nucleated cell counts were observed at 24 hours post-LPS in mice treated with 100 or 300 mg/kg Compound 1 as compared to the vehicle disease control group.

FIG. 4 compares the mean cytokines (e.g., IL-6 and TNFα) for mice receiving different treatments. Significantly increased IL-6 and TNFα levels were observed at 24 hours post-LPS in mice treated with 100 mg/kg Compound 1 as compared to the vehicle disease control group.

FIG. 5 compares the mean peribronchiolar lymphoid inflammation width for mice receiving different treatments.

FIG. 6 compares the mean histopathology (e.g., neutrophil, interstitial inflammation score, peribronchiolar/perivascular edema, and airway epithelial damage) for mice receiving different treatments. Significantly reduced Neutrophil and interstitial inflammation scores were observed at 6 hours post-LPS in mice treated with 100 or 300 mg/kg Compound 1, and observed at 24 hours post-LPS in mice treated with 300 mg/kg Compound 1, as compared to the respective vehicle disease control groups.

FIG. 7 compares the mean summed scores for mice receiving different treatments. Significantly reduced Summed lung scores were observed at 6 hours post-LPS in mice treated with 100 or 300 mg/kg Compound 1, and observed at 24 hours post-LPS in mice treated with 300 mg/kg Compound 1, as compared to the respective vehicle disease control groups.

### DETAILED DESCRIPTION OF THE INVENTION

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference for all purposes.

Animal: As used herein, the term “animal” refers to any member of the animal kingdom. In some embodiments, “animal” refers to humans, at any stage of development. In some embodiments, “animal” refers to non-human animals, at any stage of development. In embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the term “biologically active” refers to a characteristic of any agent that has activity in a biological system, and particularly in an organism. For instance, an agent that, when administered to an organism, has a biological effect on that organism, is considered to be biologically active.

Dose(s): As used herein, the term “dose(s)” means a quantity of the compound or a pharmaceutically acceptable salt, solvate, or hydrate thereof to be administered at one time. A dose may comprise a single unit dosage form, or alternatively may comprise more than a single unit dosage form (e.g., a single dose may comprise two tablets), or even less than a single unit dosage form (e.g., a single dose may comprise half of a tablet).

Daily dose: As used herein, the term “daily dose” means a quantity of the compound, or a pharmaceutically acceptable salt, solvate, or hydrate thereof that is administered in a 24-hour period. Accordingly, a daily dose may be administered all at once (i.e., once daily dosing) or alternatively the daily dosing may be divided such that administration of the compound is twice daily, three times daily, or even four times daily.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control sample or subject (or multiple control samples or subjects) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Administration: As used herein, the term “administration” typically refers to the administration of a composition to a subject or system. Those of ordinary skill in the art will be aware of a variety of routes that may, in appropriate circumstances, be utilized for administration to a subject, for example a human subject. For example, in embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In embodiments, administration may involve dosing that is intermittent (e.g., a plurality of doses separated in time) and/or periodic (e.g., individual doses separated by a common period of time) dosing. In embodiments, administration may involve continuous dosing (e.g., perfusion) for at least a selected period of time.

Hospitalization: The term “hospitalization”, as used herein, is defined as an overnight admission with observation of a minimum of 24 hours.

Pharmaceutically acceptable: The term “pharmaceutically acceptable”, as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Subject and Patient: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. As used herein, the term “patient” refers to a human. In embodiments, a patient may be suffering from, and/or susceptible to a disease, disorder, and/or condition. A patient can be afflicted with or is susceptible to a disease or disorder but may or may not display symptoms of the disease or disorder. A subject can be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one-unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Preventing: The term “prevent,” “preventing,” or “prevention,” as used herein refers to an effect that mitigates an undesired effect, e.g., an undesirable drug-drug interaction or the formation of a drug-iron chelate. Prevention does not require the 100% elimination of the possibility of an event. Rather, it denotes that the likelihood of the occurrence of the event has been reduced by the compound or method.

As used herein, the term “HIF prolyl hydroxylase” is art-recognized and may be abbreviated as “PHD”. HIF prolyl hydroxylase is also known as “prolyl hydroxylase domain-containing protein” which may be abbreviated as “PHD.” In this regard, there are three different PHD isoforms, PHD1, PHD2, and PHD3, also referred to as EGLN2, EGLN1, and EGLN3, or HPH3, HPH2, and HPH1, respectively.

Alkyl: As used herein, the term “alkyl” means acyclic linear and branched hydrocarbon groups, e.g. “C₁-C₂₀ alkyl” refers to alkyl groups having 1-20 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentyl, hexyl, isohexyl, and the like. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, haloalkyl (e.g., —CH₂F and —CH₂Cl, as well as perhaloalkyl groups such as CF₃ and CCl₃), —CO₂R′, —CN, —OH, —OR′, —NH₂, —NHR′, —N(R′)₂, —SR′ or—SO₂R′, wherein each instance of R′ independently is C₁-C₃ alkyl. In embodiments, the alkyl is unsubstituted. In embodiments, the alkyl is substituted (e.g., with 1, 2, 3, 4, 5, or 6 substituent groups as described herein).

Alkoxy: The term “alkoxy,” as used herein, a monovalent substituent which consists of a linear or branched, substituted or unsubstituted, acyclic or cyclic alkyl group linked through an ether oxygen and having its free valence bond from the ether oxygen such as, for example methoxy, ethoxy, 1- and 2-propoxy, 1-butoxy, 1,2-dimethylethoxy, 1- and 2-pentoxy-, 3-hexoxy-and the like.

Alkylene: The term “alkylene,” as used herein, represents a saturated divalent straight or branched chain hydrocarbon group, e.g., a “C₁-C₁₀ alkylene” group having 1-10 carbons. Alkylene groups are exemplified by methylene, ethylene, isopropylene and the like. An alkylene may be unsubstituted or substituted with substituent groups as described herein. In embodiments, an alkylene group is unsubstituted

Aryl: The terms “aryl” and “ar-”, used alone or as part of a larger moiety, e.g., “aralkyl”, “aralkoxy”, or “aryloxyalkyl”, refer to an optionally substituted C₆₋₁₄ aromatic hydrocarbon moiety comprising one to three aromatic rings. For example, the aryl group is a C₆₋₁₀ aryl group (i.e., phenyl and naphthyl). Aryl groups include, without limitation, optionally substituted phenyl, naphthyl, or anthracenyl. The terms “aryl” and “ar-”, as used herein, also include groups in which an aryl ring is fused to one or more cycloaliphatic rings to form an optionally substituted cyclic structure such as a tetrahydronaphthyl, indenyl, or indanyl ring. The term “aryl” may be used interchangeably with the terms “aryl group”, “aryl ring”, and “aromatic ring”.

Benzyloxy: As used herein, the term “benzyloxy” refers to a monovalent substituent which consists of a substituted or unsubstituted benzyl group linked through an ether oxygen and having its free valence bond from the ether oxygen.

Cycloalkyl: As used herein, the term “cycloalkyl” means a nonaromatic, saturated, cyclic group, e.g. “C₃-C₁₀ cycloalkyl.” In embodiments, a heterocyclyl is monocyclic. In embodiments, a heterocyclyl is polycyclic (e.g., bicyclic or tricyclic). In polycyclic cycloalkyl groups, individual rings can be fused, bridged, or spirocyclic. Examples of a cycloalkyl group include cyclopropyl, cyclobutyl, cyclopentyl, cyclohexyl, norbornanyl, bicyclo[3.2.1]octanyl, octahydro-pentalenyl, and spiro[4.5]decanyl, and the like. The term “cycloalkyl” may be used interchangeably with the term “carbocycle”. A cycloalkyl group may be unsubstituted or substituted with one or more substituent groups as described herein. For example, a cycloalkyl group may be substituted with one or more (e.g., 1, 2, 3, 4, 5, or 6 independently selected substituents) of halogen,—CO₂R′, —CN, —OH, —OR′, —NH₂, —NHR′, —N(R′)₂, —SR′ or—SO₂R′, wherein each instance of R′ independently is C₁-C₃ alkyl. In embodiments, the cycloalkyl is unsubstituted. In embodiments, the cycloalkyl is substituted (e.g., with 1, 2, 3, 4, 5, or 6 substituent groups as described herein).

Halo or Halogen: As used herein, the term “halo” or “halogen” means fluorine, chlorine, bromine, or iodine.

Heterocyclyl: As used herein, the term “heterocyclyl” means a nonaromatic, cyclic structure having at least one of any type of heteroatom as ring atoms, having any degree of unsaturation, and excludes aromatic heterocyclic rings that are defined as “heteroaryl” herein. For example, a “3- to 10-membered heterocyclyl” refers to heterocyclics having 3-10 ring atoms that are carbon or heteroatoms as described herein. The one or more heteroatoms may be selected from nitrogen, sulfur, and oxygen. The term “heterocycle”, “heterocyclyl”, “heterocyclic”, “heterocycloalkyl,” and “heterocyclic ring” can be used interchangeably. A heterocyclyl group can be attached as a substituent via a carbon atom or a heteroatom (e.g. a nitrogen atom). In embodiments, a heterocyclyl is monocyclic. In embodiments, a heterocyclyl is polycyclic (e.g., bicyclic or tricyclic). Examples include 2-tetrahydrofuranyl, 3-tetrahydrofuranyl, dihydrofuranyl, tetrahydropyranyl, pyranyl, thiopyranyl, 2-tetrahydrothiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 1,3-oxazoIidin-3-yI, isothiazolidinyl, 1,3-thiazolidin-3-yl, 1,2-pyrazolidin-2-yl, 1,3-pyrazolidin-1-yl, 1,2- tetrahydrothiazin-2-yl, 1,3-tetrahydrothiazin-3-yl, tetrahydrothiadiazinyl, 1,2- tetrahydrodiazin-2-yl, 1,3-tetrahydrodiazin-1-yl, tetrahydroazepinyl, piperizin-2-onyl, piperizin-3-onyl, 2-pyrrolinyl, 3-pyrrolinyl, imidazolidinyl, 2-imidazolidinyl, 1,4- dioxanyl, and 4-thiazolidinyl, and the like. A heterocyclyl group may be unsubstituted or substituted with one or more substituent groups as described herein. For example, a heterocyclyl group may be substituted with one or more (e.g., 1, 2, 3, 4, 5, or 6 independently selected substituents) of halogen,—CO₂R′, —CN, —OH, —OR′, —NH₂, —NHR′, —N(R′)₂, —SR′ or—SO₂R′, wherein each instance of R′ independently is C₁-C₃ alkyl. In embodiments, the heterocyclyl is unsubstituted. In embodiments, the heterocyclyl is substituted (e.g., with 1, 2, 3, 4, 5, or 6 substituent groups as described herein).

Heteroaryl: As used herein, the term “heteroaryl” means an aromatic moiety having at least one of any type of heteroatom as ring atoms. For example, a “5- to 14-membered heteroaryl” refers to heteroaryls having 5-14 ring atoms that are carbon or heteroatoms as described herein. The one or more heteroatoms may be selected from nitrogen, sulfur, and oxygen. A heteroaryl group can be attached as a substituent via a carbon atom or a heteroatom (e.g. a nitrogen atom). In embodiments, a heteroaryl is monocyclic. In embodiments, a heteroaryl is polycyclic (e.g., bicyclic or tricyclic). In embodiments, polycyclic heteroaryls comprise a cyclic group that is nonaromatic (e.g., a heteroaryl fused to a cycloalkyl or a heterocyclyl group as described herein). The term “heteroaryl” may be used interchangeably with the term “heteroaryl ring” or the term “heteroaromatic”. Examples include 5-membered monocyclic rings such as pyrrolyl, imidazolyl, pyrazolyl, triazolyl, furyl, oxazolyl, isoxazolyl, oxadiazolyl, thiadiazolyl, thiazolyl, isothiazolyl, and the like; and 6-membered monocyclic rings such as pyridyl, pyrazinyl, pyrimidinyl, pyridazinyl, triazinyl and the like. For further examples, see, e.g., Katritzky, Handbook of Heterocyclic Chemistry. Further specific examples of heteroaryl rings include 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl and carbazolyl. The term “heteroaryl” also refers to rings that are optionally substituted. A heteroaryl group may be optionally substituted with one or more functional groups discussed below. Still other examples include indolyl, azaindolyl, benzimidazolyl, indazolyl, imidazopyridinyl, imidazopyrimidinyl, pyrrolopyrimidinyl, pyrrolopyridinyl, pyrazolopyridinyl, pyrazolopyrimidinyl, quinolyl, isoquinolyl, benzoxazolyl, benzathiazolyl, benzothiophenyl, benzofuranyl, and isobenzofuranyl, and the like. A heteroaryl group may be unsubstituted or substituted with one or more substituent groups as described herein. For example, a heteroaryl group may be substituted with one or more (e.g., 1, 2, 3, 4, 5, or 6 independently selected substituents) of halogen,—CO₂R′, —CN, —OH, —OR′, —NH₂, —NHR′, —N(R′)₂, —SR′ or—SO₂R′, wherein each instance of R′ independently is C₁-C₃ alkyl. In embodiments, the heteroaryl is unsubstituted. In embodiments, the heteroaryl is substituted (e.g., with 1, 2, 3, 4, 5, or 6 substituent groups as described herein).

### Hypoxia-Inducible Factor (HIF) and HIF Prolyl 4-Hydroxylases (HIF PH)

Described herein are therapeutic methods comprising administering to a patient in need thereof a compound that is a HIF prolyl hydroxylase inhibitor or a HIF-alpha stabilizer, or a pharmaceutically acceptable salt thereof.

Hypoxia-inducible factor (HIF) is a heterodimeric transcription factor consisting of an oxygen-sensitive HIF-α subunit and a constitutively expressed HIF-β subunit. Under normoxic conditions, the HIF-α subunit is produced and continuously degraded with a t½ of approximately 5 minutes. In response to hypoxic conditions, levels of HIFα are elevated in most cells because of a decrease in HIFα prolyl hydroxylation.

Prolyl hydroxylation of HIFα is accomplished by a family of proteins variously termed the prolyl hydroxylase domain-containing proteins (PHD1, 2, and 3), also known as HIF prolyl hydroxylases (HIF-PH-3, 2, and 1) or EGLN-2, 1, and 3, which use physiological levels of oxygen to hydroxylate specific proline residues of HIF-α, signaling proteasomal degradation of HIF-α (Ke et al. (2006) Mol. Pharmacol. 70 (5): 1469-80). Under hypoxic conditions the activity of HIF-PHs is decreased, degradation of HIF-α is inhibited and cellular levels of HIF-α are increased. Stabilization of HIF-α allows it to translocate to the nucleus and dimerize with the HIF-β subunit where it binds to cis-acting hypoxia-response elements and activates the transcription of target genes involved in erythropoiesis and iron homeostasis. The HIF-PHs thus function as critical sensors of cellular oxygen levels, and regulate oxygen homeostasis in response to hypoxia through inhibition of hydroxylation thus leading to stabilization of HIF-α, which in turn signals gene transcription and translation of proteins necessary for the cellular and organ responses to poor oxygenation (Myllylharju et al. (2013) Acta Physiol 208 (2):148-65).

HIF Prolyl 4-Hydroxylases (HIF-PH), are 2-oxoglutarate-dependent dioxygenases that require oxygen for hydroxylation of proline residues in the oxygen-dependent degradation domain of HIF α, and also require Fe²⁺ and ascorbate as essential cofactors. Each of the 3 known HIF-PHs regulate HIF 1α and HIF 2α in a non-redundant manner that is dependent upon the degree of hypoxia and the relative abundance of the specific HIF-PH in different tissues. PHD2 is the abundant isoform present in most cells during normoxia (Appelhoff 2004). During hypoxia, messenger ribonucleic acid (mRNA) and protein for PHD2 and 3 were shown to increase with marked upregulation of PHD3 (Appelhoff 2004). Although all 3 HIF-PHs contribute to the regulation of HIF 1α and HIF 2α, PHD2 was shown to preferentially interact with HIF-1α, while PHD3 and PHD1 preferentially inhibit HIF 2α (Appelhoff et al. (2004) J. Biol. Chem. 279 (37):38458-65).

### Compounds of the Invention

Provided herein are exemplary compounds for use in methods for treating and/or preventing a disease or condition, methods for reducing mortality and morbidity related to a disease or condition, and methods for reducing incidence, severity, or risk of a disease or condition, wherein the disease or condition in is as described anywhere herein. Compounds that can be used with the compositions and formulations provided herein are HIF prolyl hydroxylase inhibitors or HIF-alpha stabilizers.

Non-limiting examples of HIF prolyl hydroxylase inhibitors or HIF-alpha stabilizers include vadadustat (AKB-6548), roxadustat (FG-4592), daprodustat (GSK-12788363), molidustat (BAY 85-3934), enarodustat (JTZ-951), or desidustat (ZYAN1), and compounds described in U.S. Pat. Nos. 8,759,345, 8,937,078, 8,796,263, 9,273,034, 8,530,404, 7,696,223, 7,629,357, 8,927,591, 8,269,008, 8,952,160, 8,952,160, 8,927,591, 8,921,389, 8,916,585, 8,703,795, 8,921,389, 7,662,854, and 9,040,522; in International Publication No. WO2020/072645; in U.S. Provisional Pat. Application Nos. 62/947,170, 62/947,179, 62/992,585, 62/992,606, and 62/992,616; and in “Recent Advances in Developing Inhibitors for Hypoxia-Inducible Factor Prolyl Hydroxylases and Their Therapeutic Implications” (Kim et al. Molecules 2015, 20, 20551-20568; see, e.g., any of the compounds described therein, including any of the compounds described in any of Tables 2, 3, or 4), each of which is hereby incorporated by reference in its entirety. In embodiments, a suitable compound is described in any of International Publication No. WO2020/072645; and in U.S. Provisional Pat. Application Nos. 62/947,170, 62/947,179, 62/992,585, 62/992,606, and 62/992,616.

Certain exemplary compounds are described herein.

### Formula (I): Vadadustat and Other Compounds

Exemplary compounds that can be used in any of the methods described herein include those described in U.S. Pat. Nos. 7,811,595, 8,343,952, 8,323,671, 8,598,210, 8,722,895, 8,940,773, and 9,598,370; and in U.S. Publication No. US 20190192494A1, each of which is incorporated by reference in its entirety. In embodiments, a compound, or pharmaceutically acceptable salt thereof, is described in any of claims 1-32 of U.S. Pat. No. 7,811,595.

In embodiments, suitable compounds include a compound having a structure according to Formula (I),

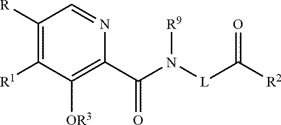

or a pharmaceutically acceptable salt thereof, wherein, R and R¹ are each independently:
(i) hydrogen
(ii) substituted or unsubstituted phenyl;
(iii) substituted or unsubstituted heteroaryl; or
(iv) substituted or unsubstituted alkyl;
said substitution selected from:
(i) C₁-C₄ alkyl;
(ii) C₃-C₄ cycloalkyl;
(iii) C₁-C₄ alkoxy;
(iv) C₃-C₄ cycloalkoxy;
(v) C₁-C₄ haloalkyl;
(vi) C₃-C4 halocycloalkyl;
(vii) halogen;
(viii) cyano;
(ix) NHC(O)R⁴;
(x) C(O)NR^{5a}R^{5b}_{;}
(xi) phenyl; and
(xii) heteroaryl; or
(xiii) two substituents are taken together to form a fused ring having from 5 to 7 atoms;
R⁴ is a C₁-C₄ alkyl or C₃-C₄ cycloalkyl;
R^{5a} and R^{5b} are each independently selected from:
   (i) hydrogen;
   (ii) C₁-C₄ alkyl;
   (iii) C₃-C₄ cycloalkyl; or
   (iv) R^{5a} and R^{5b} are taken together to form a ring having from 3 to 7 atoms;
R² is selected from:
   (i) OR⁶
   (ii) NR^{7a}R^{7b}; and
R⁶ is selected from hydrogen and C₁-C₄ alkyl or C₃-C₄ cycloalkyl;
R^{7a} and R^{7b} are each independently selected from:
   (i) hydrogen;
   (ii) C₁-C₄ alkyl or C₃-C₄ cycloalkyl; or
   (iii) R^{7a} and R^{7b} are taken together to form a ring having from 3 to 7 atoms;
R³ is selected from hydrogen, methyl, and ethyl;
L is a linking unit having a structure -[C(R^{8a}R^{8b})]ₙ-
R^{8a} and R^{8b} are each independently selected from hydrogen, methyl and ethyl;
n is an integer from 1 to 3; and
R⁹ is selected from hydrogen and methyl.

In embodiments, R is hydrogen.

In embodiments, R is unsubstituted phenyl. In embodiments, R is substituted phenyl. In embodiments, R is phenyl substituted with C₁-C₄ alkyl. In embodiments, R is phenyl substituted with C₃-C₄ cycloalkyl. In embodiments, R is phenyl substituted with C₁-C₄ alkoxy. In embodiments, R is phenyl substituted with C₃-C₄ cycloalkoxy. In embodiments, R is phenyl substituted with C₁-C₄ haloalkyl. In embodiments, R is phenyl substituted with C₃-C₄ halocycloalkyl. In embodiments, R is phenyl substituted with halogen. In embodiments, R is phenyl substituted with cyano. In embodiments, R is phenyl substituted with NHC(O)R⁴, wherein R⁴ is a C₁-C₄ alkyl or C₃-C₄ cycloalkyl. In embodiments, R is phenyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is phenyl substituted with phenyl. In embodiments, R is phenyl substituted with heteroaryl. In embodiments, R is phenyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is unsubstituted heteroaryl. In embodiments, R is substituted heteroaryl. In embodiments, R is heteroaryl substituted with C₁-C₄ alkyl. In embodiments, R is heteroaryl substituted with C₃-C₄ cycloalkyl. In embodiments, R is heteroaryl substituted with C₁-C₄ alkoxy. In embodiments, R is heteroaryl substituted with C₃-C₄ cycloalkoxy. In embodiments, R is heteroaryl substituted with C₁-C₄ haloalkyl. In embodiments, R is heteroaryl substituted with C₃-C₄ halocycloalkyl. In embodiments, R is heteroaryl substituted with halogen. In embodiments, R is heteroaryl substituted with cyano. In embodiments, R is heteroaryl substituted with NHC(O)R⁴, wherein R⁴ is a C₁-C₄ alkyl or C₃-C₄ cycloalkyl. In embodiments, R is heteroaryl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is heteroaryl substituted with phenyl. In embodiments, R is heteroaryl substituted with heteroaryl. In embodiments, R is heteroaryl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is unsubstituted alkyl. In embodiments, R is substituted alkyl. In embodiments, R is alkyl substituted with C₁-C₄ alkyl. In embodiments, R is alkyl substituted with C₃-C₄ cycloalkyl. In embodiments, R is alkyl substituted with C₁-C₄ alkoxy. In embodiments, R is alkyl substituted with C₃-C₄ cycloalkoxy. In embodiments, R is alkyl substituted with C₁-C₄ haloalkyl. In embodiments, R is alkyl substituted with C₃-C₄ halocycloalkyl. In embodiments, R is alkyl substituted with halogen. In embodiments, R is alkyl substituted with cyano. In embodiments, R is alkyl substituted with NHC(O)R⁴, wherein R⁴ is a C₁-C₄ alkyl or C₃-C₄ cycloalkyl. In embodiments, R is alkyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is alkyl substituted with phenyl. In embodiments, R is alkyl substituted with heteroaryl. In embodiments, R is alkyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is substituted or unsubstituted phenyl. In embodiments, R is chosen from 2-fluorophenyl, 3-fluorophenyl, 4-fluorophenyl, 2-chlorophenyl, 3-chlorophenyl, and 4-chlorophenyl. In embodiments, R is chosen from 2-methoxyphenyl, 3-methoxyphenyl, 4-methoxyphenyl, 2-ethoxyphenyl, 3-ethoxyphenyl, 4-ethoxyphenyl, 2-iso-propoxyphenyl, 3-iso-propoxyphenyl, 4-iso-propoxyphenyl, 2-cyanophenyl, 3-cyanophenyl, and 4-cyanophenyl.

In embodiments, R is a unit having the formula:

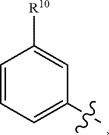

, where exemplary groups for R¹⁰ are as described herein.

In embodiments, R¹⁰ has the formula —C(O)NR^{5a}R^{5b}; R^{5a} and R^{5b}are each hydrogen or R^{5a} and R^{5b} are taken together to from a ring having 5 or 6 atoms. In embodiments, R^{5a} and R^{5b} are taken together to form a pyrrolidin-1-yl ring.

In embodiments, R¹⁰ has the formula —NHC(O)R⁴; R⁴ is a unit chosen from methyl, ethyl, n-propyl, iso-propyl, and cyclopropyl.

In embodiments, R¹⁰ is a heteroaryl unit chosen from 1,2,3,4-tetrazol-5-yl, [1,2,4]triazol-5-yl, imidazol-2-yl, furan-2-yl, furan-3-yl, thiophene-2-yl, and thiophene-3-yl.

In embodiments, R is substituted or unsubstituted heteroaryl. In embodiments, R is a unit chosen from pyridin-2-yl, pyridin-3-yl, pyridin-4-yl, pyrimidin-2-yl, pyrimidin-4-yl, pyrimidin-5-yl, isoquinolin-1-yl, isoquinolin-3-yl, and isoquinolin-4-yl.

In embodiments, R is a unit chosen from thiazol-2-yl, thiazol-4-yl, 1,2,3,4-tetrazol-5-yl, [1,2,4]triazol-5-yl, imidazol-2-yl, furan-2-yl, furan-3-yl, thiophene-2-yl, and thiophene-3-yl.

In embodiments, R¹ is hydrogen.

In embodiments, R¹ is unsubstituted phenyl. In embodiments, R¹ is substituted phenyl. In embodiments, R¹ is phenyl substituted with C₁-C₄ alkyl. In embodiments, R¹ is phenyl substituted with C₃-C₄ cycloalkyl. In embodiments, R¹ is phenyl substituted with C₁-C₄ alkoxy. In embodiments, R¹ is phenyl substituted with C₃-C₄ cycloalkoxy. In embodiments, R¹ is phenyl substituted with C₁-C₄ haloalkyl. In embodiments, R¹ is phenyl substituted with C₃-C₄ halocycloalkyl. In embodiments, R¹ is phenyl substituted with halogen. In embodiments, R¹ is phenyl substituted with cyano. In embodiments, R¹ is phenyl substituted with NHC(O)R⁴, wherein R⁴ is a C₁-C₄ alkyl or C₃-C₄ cycloalkyl. In embodiments, R¹ is phenyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is phenyl substituted with phenyl. In embodiments, R¹ is phenyl substituted with heteroaryl. In embodiments, R¹ is phenyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is unsubstituted heteroaryl. In embodiments, R¹ is substituted heteroaryl. In embodiments, R¹ is heteroaryl substituted with C₁-C₄ alkyl. In embodiments, R¹ is heteroaryl substituted with C₃-C₄ cycloalkyl. In embodiments, R¹ is heteroaryl substituted with C₁-C₄ alkoxy. In embodiments, R¹ is heteroaryl substituted with C₃-C₄ cycloalkoxy. In embodiments, R¹ is heteroaryl substituted with C₁-C₄ haloalkyl. In embodiments, R¹ is heteroaryl substituted with C₃-C₄ halocycloalkyl. In embodiments, R¹ is heteroaryl substituted with halogen. In embodiments, R¹ is heteroaryl substituted with cyano. In embodiments, R¹ is heteroaryl substituted with NHC(O)R⁴, wherein R⁴ is a C₁-C₄ alkyl or C₃-C₄ cycloalkyl. In embodiments, R¹ is heteroaryl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is heteroaryl substituted with phenyl. In embodiments, R¹ is heteroaryl substituted with heteroaryl. In embodiments, R¹ is heteroaryl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is unsubstituted alkyl. In embodiments, R¹ is substituted alkyl. In embodiments, R¹ is alkyl substituted with C₁-C₄ alkyl. In embodiments, R¹ is alkyl substituted with C₃-C₄ cycloalkyl. In embodiments, R¹ is alkyl substituted with C₁-C₄ alkoxy. In embodiments, R¹ is alkyl substituted with C₃-C₄ cycloalkoxy. In embodiments, R¹ is alkyl substituted with C₁-C₄ haloalkyl. In embodiments, R¹ is alkyl substituted with C₃-C₄ halocycloalkyl. In embodiments, R¹ is alkyl substituted with halogen. In embodiments, R¹ is alkyl substituted with cyano. In embodiments, R¹ is alkyl substituted with NHC(O)R⁴, wherein R⁴ is a C₁-C₄ alkyl or C₃-C₄ cycloalkyl. In embodiments, R¹ is alkyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is alkyl substituted with phenyl. In embodiments, R¹ is alkyl substituted with heteroaryl. In embodiments, R¹ is alkyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is chosen from 2-methoxyphenyl, 3-methoxyphenyl, 4-methoxyphenyl, 2-ethoxyphenyl, 3-ethoxyphenyl, 4-ethoxyphenyl, 2-iso-propoxyphenyl, 3-iso-propoxyphenyl, 4-iso-propoxyphenyl, 2-cyanophenyl, 3-cyanophenyl, and 4-cyanophenyl.

In embodiments, R² is OR⁶, wherein R⁶ is hydrogen, C₁-C₄ alkyl or C₃-C₄ cycloalkyl. In embodiments, R² is NR^{7a}R^{7b}, wherein R^{7a} and R^{7b} are each independently selected from the group consisting of hydrogen, C₁-C₄ alkyl, C₃-C₄ cycloalkyl, or R^{7a} and R^{7b} are taken together to form a ring having from 3 to 7 atoms.

In embodiments, R⁶ is hydrogen. In embodiments, R⁶ is methyl.

In embodiments, R² is —NR^{7a}R^{7b}; R^{7a} and R^{7b} are each independently hydrogen, methyl, or ethyl. In embodiments, R² is chosen from —NH₂, —NHCH₃, and —N(CH₃)₂.

In embodiments, R³ is hydrogen. In embodiments, R³ is methyl. In embodiments, R³ is ethyl.

In embodiments, L is a linking unit having a structure -[C(R^{8a}R^{8b})]ₙ-, wherein R^{8a} and R^{8b} are each independently selected from the group consisting of hydrogen, methyl and ethyl.

In embodiments, L is chosen from —CH₂—, —CH₂CH₂—, and —C(CH₃)₂—. In embodiments, L is —CH₂—. In embodiments, L is —CH₂CH₂—. In embodiments, L is —C(CH₃)₂—.

In embodiments, n is an integer from 1 to 3. In embodiments, n is 1. In embodiments, n is 2. In embodiments, n is 3.

In embodiments, R⁹ is hydrogen. In embodiments, R⁹ is methyl.

In embodiments, a compound according to Formula (I) has the following structure:

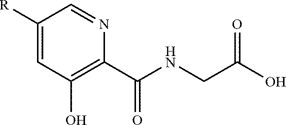

or a pharmaceutically acceptable salt thereof, wherein
R is as defined anywhere herein.

In embodiments, a compound according to Formula (I) has the following structure:

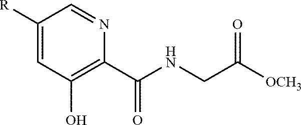

or a pharmaceutically acceptable salt thereof, wherein
R is as defined anywhere herein.

In embodiments, a compound according to Formula (I) has the following structure:

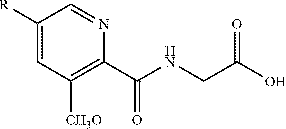

or a pharmaceutically acceptable salt thereof, wherein
R is as defined anywhere herein.

In embodiments, R is a heteroaryl unit chosen from thiazol-2-yl, thiazol-4-yl, 1,2,3,4-tetrazol-5-yl, [1,2,4]triazol-5-yl, imidazol-2-yl, furan-2-yl, furan-3-yl, thiophene-2-yl, thiophene-3-yl, pyridin-2-yl, pyridin-3-yl, pyridin-4-yl, pyrimidin-2-yl, pyrimidin-4-yl, pyrimidin-5-yl, isoquinolin-1-yl, isoquinolin-3-yl, and isoquinolin-4-yl.

In embodiments, a compound according to Formula (I) has the following structure:

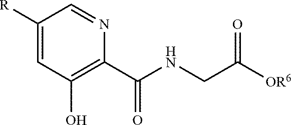

or a pharmaceutically acceptable salt thereof, wherein
R is selected from 2-chlorophenyl, 3-chlorophenyl, 4-chlorophenyl, 2-cyanophenyl, 3-cyanophenyl, and 4-cyanophenyl; and
R⁶ is selected from hydrogen, methyl, and ethyl.

In embodiments, a compound is selected from the group consisting of:
{[5-(3-Chloro-phenyl)-3-hydroxy-pyridine-2-carbonyl]-amino}-acetic acid methyl ester;
{[5-(4-Chloro-phenyl)-3-hydroxy-pyridine-2-carbonyl]-amino}-acetic acid methyl ester;
{[5-(2-Chloro-phenyl)-3-hydroxy-pyridine-2-carbonyl]-amino}-acetic acid methyl ester;
{[5-(4-Fluoro-phenyl)-3-hydroxy-pyridine-2-carbonyl]-amino}-acetic acid methyl ester;
[(3-Hydroxy-5-(4-methylphenyl)-pyridine-2-carbonyl)-amino]-acetic acid methyl ester;
{[3-Hydroxy-5-(4-isopropyl-phenyl)-pyridine-2-carbonyl]-amino}-acetic acid methyl ester;
{[5-(4-Ethyl-phenyl)-3-hydroxy-pyridine-2-carbonyl]-amino}-acetic acid methyl ester;
{[3-Hydroxy-5-(3-trifluoromethyl-phenyl)-pyridine-2-carbonyl]-amino}-acetic acid methyl ester;
{[5-(4-Cyano-phenyl)-3-hydroxy-pyridine-2-carbonyl]-amino}-acetic acid methyl ester;
{[5-(3-Cyano-phenyl)-3-hydroxy-pyridine-2-carbonyl]-amino}-acetic acid methyl ester;
{[5-(3-Carbamoyl-phenyl)-3-hydroxy-pyridine-2-carbonyl]-amino}-acetic acid methyl ester;
({3-Hydroxy-5-[3-(pyrrolidine-1-carbonyl)-phenyl]-pyridine-2-carbonyl}-amino)-acetic acid methyl ester;
({5-[3-(Cyclopropanecarbonyl-amino)-phenyl]-3-hydroxy-pyridine-2-carbonyl}-amino)-acetic acid methyl ester;
({3-Hydroxy-5-[3-(2H-tetrazol-5-yl)-phenyl]-pyridine-2-carbonyl}-amino)-acetic acid methyl ester;
[(5-Hydroxy-[3,3′]bipyridinyl-6-carbonyl)-amino]-acetic acid methyl ester
[(5′-Hydroxy-[2,3′]bipyridinyl-6′-carbonyl)-amino]-acetic acid methyl ester;
[(3-Hydroxy-5-pyrimidin-5-yl-pyridine-2-carbonyl)-amino]-acetic acid methyl ester;
[(3-Hydroxy-5-isoquinolin-4-yl-pyridine-2-carbonyl)-amino]-acetic acid methyl ester;
[(3-Hydroxy-5-thiazol-2-yl-pyridine-2-carbonyl)-amino]-acetic acid methyl ester;
{[5-(3-Chlorophenyl)-3-hydroxypyridine-2-carbonyl]amino}-acetic acid;
{[5-(4-Chlorophenyl)-3-hydroxypyridine-2-carbonyl]amino}-acetic acid;
{[5-(2-Chlorophenyl)-3-hydroxypyridine-2-carbonyl]amino}-acetic acid;
{[5-(4-Fluorophenyl)-3-hydroxypyridine-2-carbonyl]amino}-acetic acid;
[(3-Hydroxy-5-(4-methylphenyl)pyridine-2-carbonyl)amino]-acetic acid;
{[5-(4-Ethylphenyl)-3-hydroxypyridine-2-carbonyl]amino}-acetic acid;
{[3-Hydroxy-5-(4-isopropylphenyl)pyridine-2-carbonyl]amino}-acetic acid;
{[3-Hydroxy-5-(3-trifluoromethylphenyl)pyridine-2-carbonyl]amino}-acetic acid;
{[5-(4-Cyanophenyl)-3-hydroxypyridine-2-carbonyl]amino}-acetic acid;
{[5-(3-Cyanophenyl)-3-hydroxypyridine-2-carbonyl]amino}-acetic acid;
{[5-(5-Chloro-2-methylphenyl)-3-hydroxypyridine-2-carbonyl]amino}-acetic acid;
{[3-Hydroxy-5-(4-isopropoxyphenyl)pyridine-2-carbonyl]amino}-acetic acid;
({5-[3-(Cyclopropanecarbonylamino)phenyl]-3-hydroxy-pyridine-2-carbonyl}-amino)-acetic acid;
({3-Hydroxy-5-[3-(pyrrolidine-1-carbonyl)phenyl]-pyridine-2-carbonyl}amino)-acetic acid;
({3-Hydroxy-5-[3-(2H-tetrazol-5-yl)phenyl]-pyridine-2-carbonyl}-amino)-acetic acid;
[(5′-Hydroxy-[2,3′]bipyridinyl-6′-carbonyl)-amino]-acetic acid;
[(5-Hydroxy-[3,3′]bipyridinyl-6-carbonyl)-amino]-acetic acid;
[(3-Hydroxy-5-pyrimidin-5-yl-pyridine-2-carbonyl)-amino]-acetic acid;
[(3-Hydroxy-5-isoquinolin-4-yl-pyridine-2-carbonyl)-amino]-acetic acid;
[(3-Hydroxy-5-thiazol-2-yl-pyridine-2-carbonyl)-amino]-acetic acid;
{[5-(2,3-Dihydro-benzo[1,4]dioxin-6-yl)-3-hydroxy-pyridine-2-carbonyl]-amino}-acetic acid;
5-(Chlorophenyl)-N-(2-amino-2-oxo-1,1-dimethylethyl)-3-hydroxylpyridin-2-yl amide;
5-(Chlorophenyl)-N-(2-amino-2-oxoethyl)-3-hydroxylpyridin-2-yl amide;
5-(Chlorophenyl)-N-(2-amino-2-oxo-1-methylethyl)-3-hydroxylpyridin-2-yl amide;
5-(4-Methylphenyl)-N-(2-methylamino-2-oxoethyl)-3-hydroxylpyridin-2-yl amide;
5-(3-Chlorophenyl)-N-(2-methylamino-2-oxoethyl)-3-hydroxylpyridin-2-yl amide;
2-{[5-(3-Chloro-phenyl)-3-hydroxy-pyridine-2-carbonyl]-amino}-2-methyl-propionic acid methyl ester;
2-{[5-(3-Chlorophenyl)-3-hydroxy-pyridine-2-carbonyl]-amino}-2-methyl-propionic acid
3-[(3-Hydroxy-5-(4-methylphenyl)-pyridine-2-carbonyl)-amino]-propionic acid ethyl ester;
3-[(3-Hydroxy-5-(3-chlorophenyl)-pyridine-2-carbonyl)-amino]-propionic acid ethyl ester;
3-{[5-(3-Chlorophenyl)-3-hydroxy-pyridine-2-carbonyl]-amino}-propionic acid;
3-[(3-Hydroxy-5-(4-methylphenyl)-pyridine-2-carbonyl)-amino]-propionic acid;
5-(Chlorophenyl)-N-(3-amino-3-oxo-1,1-dimethylpropyl)-3-hydroxylpyridin-2-yl amide; and
5-(3-Chlorophenyl)-N-(2-dimethylamino-2-oxoethyl)-3-hydroxylpyridin-2-yl amide; or
a pharmaceutically acceptable salt thereof.

In embodiments, compounds are salts comprising anions chosen from chloride, bromide, iodide, sulfate, bisulfate, carbonate, bicarbonate, phosphate, formate, acetate, propionate, butyrate, pyruvate, lactate, oxalate, malonate, maleate, succinate, tartrate, fumarate, and citrate.

In embodiments, compounds are salts comprising cations chosen from sodium, lithium, potassium, calcium, magnesium, and bismuth.

In embodiments, a compound is {[5-(3-chlorophenyl)-3-hydroxypyridine-2-carbonyl]amino}acetic acid (Compound 1), or a pharmaceutically acceptable salt thereof. Compound 1, also referred to as vadadustat or AKB-6548, has the following structure:

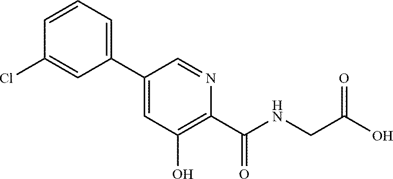 [SMILES: O=C(O)CNC(=O)c1ncc(-c2cccc(Cl)c2)cc1O]

In embodiments, a compound is {[5-(3-fluorophenyl)-3-hydroxypyridine-2-carbonyl]amino}acetic acid (Compound 1a), or a pharmaceutically acceptable salt thereof. Compound 1a, also referred to as AKB-6899, has the following structure:

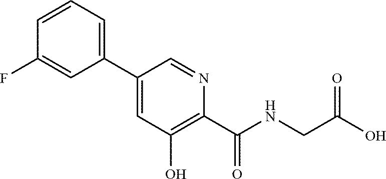 [SMILES: O=C(O)CNC(=O)c1ncc(-c2cccc(F)c2)cc1O]

### Formula (II): Enarodustat and Other Compounds

Exemplary compounds that can be used in any of the methods described herein include those described in U.S. Pat. No. 8,283,465, U.S. Publication No. US20160145254A1, and U.S. Publication No. US20200017492A1, each of which is incorporated by reference in its entirety. In embodiments, a compound, or pharmaceutically acceptable salt thereof, is described in any of claims 1-30 of U.S. Pat. No. 8,283,465.

In embodiments, suitable compounds include a compound having a structure according to Formula (II),

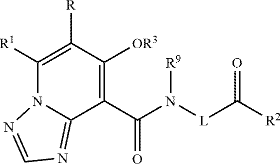

or a pharmaceutically acceptable salt thereof, wherein R and R¹ are each independently:
(i) hydrogen
(ii) substituted or unsubstituted phenyl;
(iii) substituted or unsubstituted heteroaryl; or
(iv) substituted or unsubstituted alkyl;
said substitution selected from:
(i) C₁-C₄ alkyl;
(ii) C₃-C₄ cycloalkyl;
(iii) C₁-C₄ alkoxy;
(iV) C₃-C₄ cycloalkoxy;
(v) C₁-C₄ haloalkyl;
(vi) C₃-C4 halocycloalkyl;
(vii) halogen;
(viii) cyano;
(ix) NHC(O)R⁴;
(x) C(O)NR^{5a}R^{5b};
(xi) phenyl; and
(xii) heteroaryl; or
(xiii) two substituents are taken together to form a fused ring having from 5 to 7 atoms;
R⁴ is a C₁-C₄ alkyl or C₃-C₄ cycloalkyl;
R^{5a} and R^{5b} are each independently selected from:
(i) hydrogen;
(ii) C₁-C₄ alkyl;
(iii) C₃-C₄ cycloalkyl; or
(iv) R^{5a} and R^{5b} are taken together to form a ring having from 3 to 7 atoms;
R² is selected from:
(i) OR⁶
(ii) NR^{7a}R^{7b}; and
R⁶ is selected from hydrogen and C₁-C₄ alkyl or C₃-C₄ cycloalkyl;
R^{7a} and R^{7b} are each independently selected from:
(i) hydrogen;
(ii) C₁-C₄ alkyl or C₃-C₄ cycloalkyl; or
(iii) R^{7a} and R^{7b} are taken together to form a ring having from 3 to 7 atoms;
R³ is selected from hydrogen, methyl, and ethyl;
L is a linking unit having a structure -[C(R^{8a}R^{8b})]ₙ-
R^{8a} and R^{8b} are each independently selected from hydrogen, methyl and ethyl;
n is an integer from 1 to 3; and
R⁹ is selected from hydrogen and methyl.

In embodiments, R is hydrogen.

In embodiments, R is unsubstituted phenyl. In embodiments, R is substituted phenyl. In embodiments, R is phenyl substituted with C₁-C₄ alkyl. In embodiments, R is phenyl substituted with C₃-C₄ cycloalkyl. In embodiments, R is phenyl substituted with C₁-C₄ alkoxy. In embodiments, R is phenyl substituted with C₃-C₄ cycloalkoxy. In embodiments, R is phenyl substituted with C₁-C₄ haloalkyl. In embodiments, R is phenyl substituted with C₃-C₄ halocycloalkyl. In embodiments, R is phenyl substituted with halogen. In embodiments, R is phenyl substituted with cyano. In embodiments, R is phenyl substituted with NHC(O)R⁴, wherein R⁴ is a C₁-C₄ alkyl or C₃-C₄ cycloalkyl. In embodiments, R is phenyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is phenyl substituted with phenyl. In embodiments, R is phenyl substituted with heteroaryl. In embodiments, R is phenyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is unsubstituted heteroaryl. In embodiments, R is substituted heteroaryl. In embodiments, R is heteroaryl substituted with C₁-C₄ alkyl. In embodiments, R is heteroaryl substituted with C₃-C₄ cycloalkyl. In embodiments, R is heteroaryl substituted with C₁-C₄ alkoxy. In embodiments, R is heteroaryl substituted with C₃-C₄ cycloalkoxy. In embodiments, R is heteroaryl substituted with C₁-C₄ haloalkyl. In embodiments, R is heteroaryl substituted with C₃-C₄ halocycloalkyl. In embodiments, R is heteroaryl substituted with halogen. In embodiments, R is heteroaryl substituted with cyano. In embodiments, R is heteroaryl substituted with NHC(O)R⁴, wherein R⁴ is a C₁-C₄ alkyl or C₃-C₄ cycloalkyl. In embodiments, R is heteroaryl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is heteroaryl substituted with phenyl. In embodiments, R is heteroaryl substituted with heteroaryl. In embodiments, R is heteroaryl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is unsubstituted alkyl. In embodiments, R is substituted alkyl. In embodiments, R is alkyl substituted with C₁-C₄ alkyl. In embodiments, R is alkyl substituted with C₃-C₄ cycloalkyl. In embodiments, R is alkyl substituted with C₁-C₄ alkoxy. In embodiments, R is alkyl substituted with C₃-C₄ cycloalkoxy. In embodiments, R is alkyl substituted with C₁-C₄ haloalkyl. In embodiments, R is alkyl substituted with C₃-C₄ halocycloalkyl. In embodiments, R is alkyl substituted with halogen. In embodiments, R is alkyl substituted with cyano. In embodiments, R is alkyl substituted with NHC(O)R⁴, wherein R⁴ is a C₁-C₄ alkyl or C₃-C₄ cycloalkyl. In embodiments, R is alkyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is alkyl substituted with phenyl. In embodiments, R is alkyl substituted with heteroaryl. In embodiments, R is alkyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is substituted or unsubstituted alkyl. In embodiments, R is a C₁-C₆ alkyl. In embodiments, R is a C₃-C₈ cycloalkyl. In embodiments, R is a C₆-C₁₄ aryl. In embodiments, R is a C₆-C₁₄ aryl. In embodiments, R is a C₆-C₁₄ aryl-C₁-C₆ alkyl group.

In embodiments, R¹ is hydrogen.

In embodiments, R¹ is unsubstituted phenyl. In embodiments, R¹ is substituted phenyl. In embodiments, R¹ is phenyl substituted with C₁-C₄ alkyl. In embodiments, R¹ is phenyl substituted with C₃-C₄ cycloalkyl. In embodiments, R¹ is phenyl substituted with C₁-C₄ alkoxy. In embodiments, R¹ is phenyl substituted with C₃-C₄ cycloalkoxy. In embodiments, R¹ is phenyl substituted with C₁-C₄ haloalkyl. In embodiments, R¹ is phenyl substituted with C₃-C₄ halocycloalkyl. In embodiments, R¹ is phenyl substituted with halogen. In embodiments, R¹ is phenyl substituted with cyano. In embodiments, R¹ is phenyl substituted with NHC(O)R⁴, wherein R⁴ is a C₁-C₄ alkyl or C₃-C₄ cycloalkyl. In embodiments, R¹ is phenyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is phenyl substituted with phenyl. In embodiments, R¹ is phenyl substituted with heteroaryl. In embodiments, R¹ is phenyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is unsubstituted heteroaryl. In embodiments, R¹ is substituted heteroaryl. In embodiments, R¹ is heteroaryl substituted with C₁-C₄ alkyl. In embodiments, R¹ is heteroaryl substituted with C₃-C₄ cycloalkyl. In embodiments, R¹ is heteroaryl substituted with C₁-C₄ alkoxy. In embodiments, R¹ is heteroaryl substituted with C₃-C₄ cycloalkoxy. In embodiments, R¹ is heteroaryl substituted with C₁-C₄ haloalkyl. In embodiments, R¹ is heteroaryl substituted with C₃-C₄ halocycloalkyl. In embodiments, R¹ is heteroaryl substituted with halogen. In embodiments, R¹ is heteroaryl substituted with cyano. In embodiments, R¹ is heteroaryl substituted with NHC(O)R⁴, wherein R⁴ is a C₁-C₄ alkyl or C₃-C₄ cycloalkyl. In embodiments, R¹ is heteroaryl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is heteroaryl substituted with phenyl. In embodiments, R¹ is heteroaryl substituted with heteroaryl. In embodiments, R¹ is heteroaryl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is unsubstituted alkyl. In embodiments, R¹ is substituted alkyl. In embodiments, R¹ is alkyl substituted with C₁-C₄ alkyl. In embodiments, R¹ is alkyl substituted with C₃-C₄ cycloalkyl. In embodiments, R¹ is alkyl substituted with C₁-C₄ alkoxy. In embodiments, R¹ is alkyl substituted with C₃-C₄ cycloalkoxy. In embodiments, R¹ is alkyl substituted with C₁-C₄ haloalkyl. In embodiments, R¹ is alkyl substituted with C₃-C₄ halocycloalkyl. In embodiments, R¹ is alkyl substituted with halogen. In embodiments, R¹ is alkyl substituted with cyano. In embodiments, R¹ is alkyl substituted with NHC(O)R⁴, wherein R⁴ is a C₁-C₄ alkyl or C₃-C₄ cycloalkyl. In embodiments, R¹ is alkyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is alkyl substituted with phenyl. In embodiments, R¹ is alkyl substituted with heteroaryl. In embodiments, R¹ is alkyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is C₁-C₁₀ alkyl. In embodiments, R¹ is C₃-C₈ unsubstituted cycloalkyl. In embodiments, R¹ is substituted C₃-C₈ cycloalkyl. In embodiments, R¹ is C₃-C₈ cycloalkyl substituted with 1 to 5 substituents independently selected from the group consisting of halogen, C₁-C₆ alkyl, C₃-C₈ cycloalkyl, cyano, and C₁-C₆ haloalkyl. In embodiments, R¹ is unsubstituted C₃-C₈ cycloalkenyl. In embodiments, R¹ is substituted C₃-C₈ cycloalkenyl. In embodiments, R¹ is C₃-C₈ cycloalkenyl substituted with 1 to 5 substituents independently selected from the group consisting of halogen, C₁-C₆ alkyl, C₃-C₈ cycloalkyl, cyano, and C₁-C₆ haloalkyl.

In embodiments, R¹ is unsubstituted C₆-C₁₄ aryl. In embodiments, R¹ is substituted C₆-C₁₄ aryl. In embodiments, R¹ is C₆-C₁₄ aryl substituted with 1 to 5 substituents independently selected from the group consisting of halogen, C₁-C₆ alkyl, C₃-C₈ cycloalkyl, cyano, and C₁-C₆ haloalkyl. In embodiments, R¹ is unsubstituted heteroaryl, wherein the heteroaryl has, besides carbon atom, 1 to 6 hetero atoms selected from nitrogen atom, oxygen atom and sulfur atom. In embodiments, R¹ is substituted C₆-C₁₄ heteroaryl, wherein the heteroaryl has, besides carbon atom, 1 to 6 hetero atoms selected from nitrogen atom, oxygen atom and sulfur atom. In embodiments, R¹ is heteroaryl substituted with 1 to 5 substituents independently selected from the group consisting of halogen, C₁-C₆ alkyl, C₃-C₈ cycloalkyl, cyano, and C₁-C₆ haloalkyl, and wherein the heteroaryl has, besides carbon atom, 1 to 6 hetero atoms selected from nitrogen atom, oxygen atom and sulfur atom.

In embodiments, R¹ is a C₆-C₁₄ aryl-C₁-C₆ alkyl group, wherein C₆-C₁₄ aryl is optionally substituted by the same or different 1 to 5 substituents selected from the group consisting of halogen, C₁-C₆ alkyl, C₃-C₈ cycloalkyl, cyano, and C₁-C₆ haloalkyl. In embodiments, R¹ is a C₃—Cs cycloalkyl-C₁-C₆ alkyl group, wherein C₃-C₈ cycloalkyl is optionally substituted with 1 to 5 substituents independently selected from the group consisting of halogen, C₁-C₆ alkyl, C₃-C₈ cycloalkyl, cyano, and C₁-C₆ haloalkyl. In embodiments, R¹ is a phenyl-C₁-C₆ alkyl group.

In embodiments, R² is OR⁶, wherein R⁶ is hydrogen, C₁-C₄ alkyl or C₃-C₄ cycloalkyl. In embodiments, R² is NR^{7a}R^{7b}, wherein R^{7a} and R^{7b} are each independently selected from the group consisting of hydrogen, C₁-C₄ alkyl, C₃-C₄ cycloalkyl, or R^{7a} and R^{7b} are taken together to form a ring having from 3 to 7 atoms.

In embodiments, R³ is hydrogen. In embodiments, R³ is methyl. In embodiments, R³ is ethyl.

In embodiments, L is a linking unit having a structure -[C(R^{8a}R^{8b})]ₙ-, wherein R^{8a} and R^{8b} are each independently selected from the group consisting of hydrogen, methyl and ethyl.

In embodiments, n is an integer from 1 to 3. In embodiments, n is 1. In embodiments, n is 2. In embodiments, n is 3.

In embodiments, R⁹ is hydrogen. In embodiments, R⁹ is methyl.

In embodiments, a compound is [(7-hydroxy-5-phenethyl [1,2,4] triazolo [1,5-a] pyridine-8-carbonyl)aminoacetic acid (Compound 2), or a pharmaceutically acceptable salt thereof. Compound 2, also referred to as enarodustat, or JTZ-951, has the following structure:

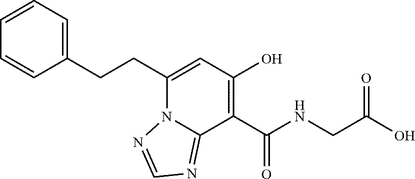 [SMILES: O=C(O)CNC(=O)c1c(O)cc(CCc2ccccc2)n2ncnc12]

### Formula (III): Desidustat and Related Compounds

Exemplary compounds that can be used in any of the methods described herein include those described in U.S. Pat. No. 9,394,300, and U.S. Publication No. US 20190359574A1, each of which is incorporated by reference in its entirety. In embodiments, a compound, or pharmaceutically acceptable salt thereof, is described in any of claims 1-10 of U.S. Pat. No. 9,394,300.

In embodiments, suitable compounds include a compound having a structure according to Formula (III),

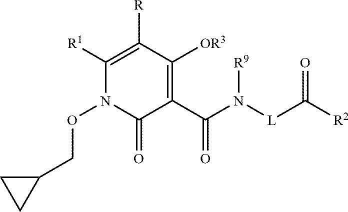

or a pharmaceutically acceptable salt thereof, wherein, R and R¹ are each independently:
(i) hydrogen
(ii) substituted or unsubstituted phenyl;
(iii) substituted or unsubstituted heteroaryl; or
(iv) substituted or unsubstituted alkyl;
said substitution selected from:
(i) C₁-C₄ alkyl;
(ii) C₃-C₄ cycloalkyl;
(iii) C₁-C₄ alkoxy;
(iv) C₃-C₄ cycloalkoxy;
(v) C₁-C₄ haloalkyl;
(vi) C₃-C4 halocycloalkyl;
(vii) halogen;
(viii) cyano;
(ix) NHC(O)R⁴;
(x) C(O)NR^{5a}R^{5b};
(xi) phenyl; and
(xii) heteroaryl; or
(xiii) two substituents are taken together to form a fused ring having from 5 to 7 atoms;
R⁴ is a C₁-C₄ alkyl or C₃-C₄ cycloalkyl;
R^{5a} and R^{5b} are each independently selected from:
(i) hydrogen;
(ii) C₁-C₄ alkyl;
(iii) C₃-C₄ cycloalkyl; or
(iv) R^{5a} and R^{5b} are taken together to form a ring having from 3 to 7 atoms;
R² is selected from:
(i) OR⁶
(ii) NR^{7a}R^{7b}; and
R⁶ is selected from hydrogen and C₁-C₄ alkyl or C₃-C₄ cycloalkyl;
R^{7a} and R^{7b} are each independently selected from:
(i) hydrogen;
(ii) C₁-C₄ alkyl or C₃-C₄ cycloalkyl; or
(iii) R^{7a} and R^{7b} are taken together to form a ring having from 3 to 7 atoms;
R³ is selected from hydrogen, methyl, and ethyl;
L is a linking unit having a structure -[C(R^{8a}R^{8b})]ₙ-
R^{8a} and R^{8b} are each independently selected from hydrogen, methyl and ethyl;
n is an integer from 1 to 3; and
R⁹ is selected from hydrogen and methyl.

In embodiments, R is hydrogen.

In embodiments, R is unsubstituted phenyl. In embodiments, R is substituted phenyl. In embodiments, R is phenyl substituted with C₁-C₄ alkyl. In embodiments, R is phenyl substituted with C₃-C₄ cycloalkyl. In embodiments, R is phenyl substituted with C₁-C₄ alkoxy. In embodiments, R is phenyl substituted with C₃-C₄ cycloalkoxy. In embodiments, R is phenyl substituted with C₁-C₄ haloalkyl. In embodiments, R is phenyl substituted with C₃-C₄ halocycloalkyl. In embodiments, R is phenyl substituted with halogen. In embodiments, R is phenyl substituted with cyano. In embodiments, R is phenyl substituted with NHC(O)R⁴, wherein R⁴ is a C₁-C₄ alkyl or C₃-C₄ cycloalkyl. In embodiments, R is phenyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is phenyl substituted with phenyl. In embodiments, R is phenyl substituted with heteroaryl. In embodiments, R is phenyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is unsubstituted heteroaryl. In embodiments, R is substituted heteroaryl. In embodiments, R is heteroaryl substituted with C₁-C₄ alkyl. In embodiments, R is heteroaryl substituted with C₃-C₄ cycloalkyl. In embodiments, R is heteroaryl substituted with C₁-C₄ alkoxy. In embodiments, R is heteroaryl substituted with C₃-C₄ cycloalkoxy. In embodiments, R is heteroaryl substituted with C₁-C₄ haloalkyl. In embodiments, R is heteroaryl substituted with C₃-C₄ halocycloalkyl. In embodiments, R is heteroaryl substituted with halogen. In embodiments, R is heteroaryl substituted with cyano. In embodiments, R is heteroaryl substituted with NHC(O)R⁴, wherein R⁴ is a C₁-C₄ alkyl or C₃-C₄ cycloalkyl. In embodiments, R is heteroaryl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is heteroaryl substituted with phenyl. In embodiments, R is heteroaryl substituted with heteroaryl. In embodiments, R is heteroaryl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R is unsubstituted alkyl. In embodiments, R is substituted alkyl. In embodiments, R is alkyl substituted with C₁-C₄ alkyl. In embodiments, R is alkyl substituted with C₃-C₄ cycloalkyl. In embodiments, R is alkyl substituted with C₁-C₄ alkoxy. In embodiments, R is alkyl substituted with C₃-C₄ cycloalkoxy. In embodiments, R is alkyl substituted with C₁-C₄ haloalkyl. In embodiments, R is alkyl substituted with C₃-C₄ halocycloalkyl. In embodiments, R is alkyl substituted with halogen. In embodiments, R is alkyl substituted with cyano. In embodiments, R is alkyl substituted with NHC(O)R⁴, wherein R⁴ is a C₁-C₄ alkyl or C₃-C₄ cycloalkyl. In embodiments, R is alkyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R is alkyl substituted with phenyl. In embodiments, R is alkyl substituted with heteroaryl. In embodiments, R is alkyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is hydrogen.

In embodiments, R¹ is unsubstituted phenyl. In embodiments, R¹ is substituted phenyl. In embodiments, R¹ is phenyl substituted with C₁-C₄ alkyl. In embodiments, R¹ is phenyl substituted with C₃-C₄ cycloalkyl. In embodiments, R¹ is phenyl substituted with C₁-C₄ alkoxy. In embodiments, R¹ is phenyl substituted with C₃-C₄ cycloalkoxy. In embodiments, R¹ is phenyl substituted with C₁-C₄ haloalkyl. In embodiments, R¹ is phenyl substituted with C₃-C₄ halocycloalkyl. In embodiments, R¹ is phenyl substituted with halogen. In embodiments, R¹ is phenyl substituted with cyano. In embodiments, R¹ is phenyl substituted with NHC(O)R⁴, wherein R⁴ is a C₁-C₄ alkyl or C₃-C₄ cycloalkyl. In embodiments, R¹ is phenyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is phenyl substituted with phenyl. In embodiments, R¹ is phenyl substituted with heteroaryl. In embodiments, R¹ is phenyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is unsubstituted heteroaryl. In embodiments, R¹ is substituted heteroaryl. In embodiments, R¹ is heteroaryl substituted with C₁-C₄ alkyl. In embodiments, R¹ is heteroaryl substituted with C₃-C₄ cycloalkyl. In embodiments, R¹ is heteroaryl substituted with C₁-C₄ alkoxy. In embodiments, R¹ is heteroaryl substituted with C₃-C₄ cycloalkoxy. In embodiments, R¹ is heteroaryl substituted with C₁-C₄ haloalkyl. In embodiments, R¹ is heteroaryl substituted with C₃-C₄ halocycloalkyl. In embodiments, R¹ is heteroaryl substituted with halogen. In embodiments, R¹ is heteroaryl substituted with cyano. In embodiments, R¹ is heteroaryl substituted with NHC(O)R⁴, wherein R⁴ is a C₁-C₄ alkyl or C₃-C₄ cycloalkyl. In embodiments, R¹ is heteroaryl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is heteroaryl substituted with phenyl. In embodiments, R¹ is heteroaryl substituted with heteroaryl. In embodiments, R¹ is heteroaryl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R¹ is unsubstituted alkyl. In embodiments, R¹ is substituted alkyl. In embodiments, R¹ is alkyl substituted with C₁-C₄ alkyl. In embodiments, R¹ is alkyl substituted with C₃-C₄ cycloalkyl. In embodiments, R¹ is alkyl substituted with C₁-C₄ alkoxy. In embodiments, R¹ is alkyl substituted with C₃-C₄ cycloalkoxy. In embodiments, R¹ is alkyl substituted with C₁-C₄ haloalkyl. In embodiments, R¹ is alkyl substituted with C₃-C₄ halocycloalkyl. In embodiments, R¹ is alkyl substituted with halogen. In embodiments, R¹ is alkyl substituted with cyano. In embodiments, R¹ is alkyl substituted with NHC(O)R⁴, wherein R⁴ is a C₁-C₄ alkyl or C₃-C₄ cycloalkyl. In embodiments, R¹ is alkyl substituted with C(O)NR^{5a}R^{5b}, wherein R^{5a} and R^{5b} are each independently selected from hydrogen, C₁-C₄ alkyl, C₃-C₄ cycloalkyl; or R^{5a} and R^{5b} are taken together to form a ring having from 3 to 7 atoms. In embodiments, R¹ is alkyl substituted with phenyl. In embodiments, R¹ is alkyl substituted with heteroaryl. In embodiments, R¹ is alkyl substituted with two substituents independently selected from the group consisting of C₁-C₄ alkyl, C₃-C₄ cycloalkyl, C₁-C₄ alkoxy, C₃-C₄ cycloalkoxy, C₁-C₄ haloalkyl, C₃-C₄ halocycloalkyl, NHC(O)R⁴, C(O)NR^{5a}R^{5b}, phenyl and heteroaryl, wherein the two substituents are taken together to form a fused ring having from 5 to 7 atoms, and wherein R⁴ is a C₁-C₄ alkyl or C₃-C₄ cycloalkyl, R^{5a} and R^{5b} are each independently selected from hydrogen, C₁-C₄ alkyl, C₃-C₄ cycloalkyl, or R^{5a} and R^{5b} are taken together to form a ring having from 3 to 7 atoms.

In embodiments, R and R¹ are taken together to form a fused ring having from 5 to 7 atoms. In embodiments, R and R¹ are taken together to form a fused ring that is an aryl. In embodiments, R and R¹ are taken together to form a fused ring that is a phenyl.

In embodiments, R² is OR⁶, wherein R⁶ is hydrogen, C₁-C₄ alkyl or C₃-C₄ cycloalkyl. In embodiments, R² is NR^{7a}R^{7b}, wherein R^{7a} and R^{7b} are each independently selected from the group consisting of hydrogen, C₁-C₄ alkyl, C₃-C₄ cycloalkyl, or R^{7a} and R^{7b} are taken together to form a ring having from 3 to 7 atoms.

In embodiments, R³ is hydrogen. In embodiments, R³ is methyl. In embodiments, R³ is ethyl.

In embodiments, L is a linking unit having a structure -[C(R^{8a}R^{8b})]ₙ-, wherein R^{8a} and R^{8b} are each independently selected from the group consisting of hydrogen, methyl and ethyl.

In embodiments, n is an integer from 1 to 3. In embodiments, n is 1. In embodiments, n is 2. In embodiments, n is 3.

In embodiments, R⁹ is hydrogen. In embodiments, R⁹ is methyl.

In embodiments, a compound is 2-(1-(cyclopropylmethoxy)-4-hydroxy-2-oxo-1,2-dihydroquinoline-3-carboxamido) acetic acid (Compound 3), or a pharmaceutically acceptable salt thereof. Compound 3, also referred to as desidustat, or ZYAN1, has the following structure:

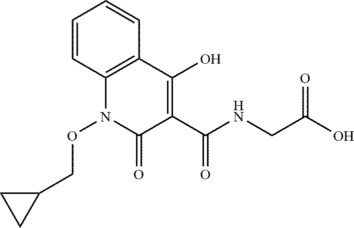 [SMILES: O=C(O)CNC(=O)c1c(O)c2ccccc2n(OCC2CC2)c1=O]

### Formula (IV): Roxadustat and Related Compounds

Still other compounds that can be useful in the methods described herein include those described in U.S. Pat. No. 7,323,475, which is incorporated by reference in its entirety. In embodiments, a compound, or a pharmaceutically acceptable salt thereof, is described in any of claims 1-46 of U.S. Pat. No. 7,323,475.

In embodiments, suitable compounds include a compound having a structure according to Formula (IV),

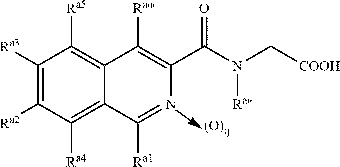

or a pharmaceutically acceptable salt thereof, wherein
q is zero or one;
R^{a1} is selected from the group consisting of hydrogen, alkyl, Substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR^{a6} where X is oxygen, — S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted hetero cyclic, and R^{a7} is hydrogen, alkyl or aryl or, when X is —NR^{a7}— , then R^{a7} and R^{a6}, together with the nitrogen atom to which they are bound, can be joined to form a heterocyclic or substituted heterocyclic group;
R^{a2} and R^{a3} are independently selected from the group consisting of hydrogen, alkyl, substituted alkyl, aryl, substituted aryl, heteroaryl, substituted heteroaryl, halo, hydroxy, cyano. —S(O)ₙ— N(R^{a6})—R^{a6} where n is 0, 1, or 2, —NR^{a6}C(O)NR^{a6}R^{a6}, —XR^{a6} where X is oxygen, —S(O)ₙ—, or — NR^{a7}— where n is zero, one or two, each R^{a6} is independently selected from the group consisting of hydrogen, alkyl, Substituted alkyl, aryl, substituted aryl, cycloalkyl, substituted cycloalkyl, heteroaryl, substituted heteroaryl, heterocyclic and substituted heterocyclic provided that when when X is —SO— or —SO₂— , then R^{a6} is not hydrogen, and R^{a7} is selected from the group consisting of hydrogen, alkyl, aryl, or R^{a2}, R^{a3} together with the carbon atom pendent thereto, form an aryl substituted aryl, heteroaryl, or substituted heteroaryl;
R^{a4} and R^{a5} are independently selected from the group consisting of hydrogen, halo, alkyl, substituted alkyl, alkoxy, Substituted alkoxy, aryl, substituted aryl, heteroaryl, substituted heteroaryl and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl or, when X is —NR^{a7}—, then R^{a7} and R^{a6}, together with the nitrogen atom to which they are bound, can be joined to form a heterocyclic or Substituted heterocyclic group;
R^{a″} is selected from the group consisting of hydrogen, alkyl and substituted alkyl;
R^{a‴} is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)—R^{a10} wherein R^{a10} is selected from the group consisting of alkyl, substituted alkyl, cycloalkyl, substituted cycloalkyl, aryl, substituted aryl, heteroaryl and substituted heteroaryl and n is zero, one or two;
or a pharmaceutically acceptable salt, ester, or prodrug thereof;
with the proviso that when R^{a″} is hydrogen and q is zero, then at least one of the following occurs:
1) R^{a1} is fluoro, bromo, iodo, alkyl, substituted alkyl, alkoxy, aminoacyl, Substituted alkoxy, aryl, substituted aryl, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl; or
2) R^{a2} is substituted alkyl, aryl, substituted aryl, heteroaryl, substituted heteroaryl, fluoro, bromo, iodo, cyano, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
a) when R^{a2} is substituted alkyl such a substituent does not include trifluoromethyl;
b) —XR^{a6} is not alkoxy; and
c) when —XR^{a6} is substituted alkoxy such a substituent does not include benzyl or benzyl substituted by a substituent selected from the group consisting of (C₁-C₅) alkyl and (C₁-C₅) alkoxy or does not include a fluoroalkoxy substituent of the formula:
—O—[CH₂]ₓ—C_{f}H(_{2f+i-g})F_{g}
where x is zero or one; f is an integer of from 1 to 5; and
g is an integer of from 1 to (2f+1); or
3) R^{a3} is substituted alkyl, aryl, substituted aryl, heteroaryl, substituted heteroaryl, bromo, iodo, —XR^{a6} where X is oxygen—S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
a) when R^{a3} is substituted alkyl such a substituent does not include trifluoromethyl;
b) —XR^{a6} is not alkoxy; and
c) when XR^{a6} is substituted alkoxy such a substituent does not include benzyl or benzyl substituted by a substituent selected from the group consisting of (C₁-C₅) alkyl and (C—C) alkoxy or does not include a fluoroalkoxy substituent of the formula:
—O—[CH₂]ₓ—C_{f}H(_{2f+i-g})F_{g}
where x is zero or one; f is an integer of from 1 to 5; and
g is an integer of from 1 to (2f+1); or
4) R^{a4} is iodo, substituted alkyl, aryl, substituted aryl, heteroaryl, substituted heteroaryl, —XR^{a6} where X is oxygen—S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
a) when R^{a4} is substituted alkyl such a substituent does not include trifluoromethyl;
b) —XR^{a6} is not alkoxy; and
c) when XR^{a6} is substituted alkoxy such a substituent does not include benzyl or benzyl substituted by a substituent selected from the group consisting of (C₁-C₅) alkyl and (C—C) alkoxy or does not include a fluoroalkoxy substituent of the formula:
—O—[CH₂]_{x—}C_{f}H_{(2f+1-g)}F_{g}
where x is zero or one; f is an integer of from 1 to 5; and
g is an integer of from 1 to (2f+1); or
5) R^{a5} is iodo, substituted alkyl, aryl, substituted aryl, heteroaryl, substituted heteroaryl, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
a) when R^{a5} is substituted alkyl such a substituent does not include trifluoromethyl;
b) —XR^{a6} is not alkoxy; and
c) when XR^{a6} is substituted alkoxy such a substituent does not include benzyl or benzyl substituted by a substituent selected from the group consisting of (C₁-C₅) alkyl and (C—C) alkoxy or does not include a fluoroalkoxy substituent of the formula:
—O—[CH₂]ₓ—C_{f}H(_{2f+i-g})F_{g}
where x is zero or one; f is an integer of from 1 to 5; and
g is an integer of from 1 to (2f+1);
and with the further following proviso:
that when R^{a1}, R^{a3}, R^{a4}, and R^{a5} are hydrogen, then R^{a2} is not bromo.

In embodiments, a compound according to Formula (IV) has the following structure:

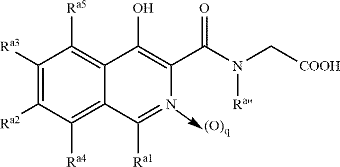

or a pharmaceutically acceptable salt thereof, wherein
q is zero or one;
R^{a″} is selected from hydrogen and alkyl:
R^{a1} is selected from the group consisting of hydrogen, alkyl, Substituted alkyl, alkoxy, substituted alkoxy, aryl, substituted aryl, halo, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, Substituted alkyl, aryl, sub stituted aryl, heteroaryl, substituted heteroaryl, heterocyclic and substituted heterocyclic, and R^{a7} is hydrogen, alkyl or aryl;
R^{a2} and R^{a3} are independently selected from the group consisting of hydrogen, alkyl, substituted alkyl, aryl, substituted aryl, heteroaryl, substituted heteroaryl, halo, hydroxy, cyano, —XR^{a6} where X is oxygen, —S(O)ₙ—, or —NR^{a7}— where n is zero, one or two, R^{a6} is selected from the group consisting of alkyl, Substituted alkyl, aryl, substituted aryl, heteroaryl, substituted heteroaryl, heterocyclic and substituted heterocyclic, and R^{a7} is hydrogen, alkyl or aryl;
R^{a4} and R^{a5} are independently selected from the group consisting of hydrogen, halo, alkyl, substituted alkyl, alkoxy, substituted alkoxy, aryl, substituted aryl, heteroaryl, substituted heteroaryl, and —XR^{a6} where X is oxygen, —S(O)ₙ—, or —NR^{a7}— where n is zero, one or two, R^{a6} is selected from the group consisting of alkyl, Substituted alkyl, aryl, substituted aryl, heteroaryl, substituted heteroaryl, heterocyclic and substituted heterocyclic, and R^{a7} is hydrogen, alkyl or aryl; or a pharmaceutically acceptable salt or prodrug thereof.

In embodiments, a compound according to Formula (IV) has the following structure:

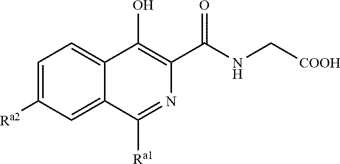

or a pharmaceutically acceptable salt thereof, wherein
R^{a1} is selected from the group consisting of hydrogen, halo, alkyl, substituted alkyl, alkoxy, substituted alkoxy, amino, substituted amino, aminoacyl, aryl, substituted aryl, heteroaryl, substituted heteroaryl, heterocyclic, substituted heterocyclic, and —XR^{a6} where X is oxygen, —S(O)ₙ—, or — NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl or, when X is —NR^{a7}— , then R^{a7} and R^{a6}, together with the nitrogen atom to which they are bound, can be joined to form a heterocyclic or substituted heterocyclic group; and
R^{a2} is selected from the group consisting of hydrogen, alkyl, substituted alkyl, aryl, substituted aryl, heteroaryl, substituted heteroaryl, halo, hydroxy, cyano, —S(O)ₙ—N(R^{a6})—R^{a6} where n is 0, 1 or 2, —NR^{a6}C(O)NR^{a6}R^{a6}, XR^{a6} where X is oxygen, —S(O)ₙ—, or —NR^{a7}— where n is zero, one or two, each R^{a6} is independently selected from the group consisting of hydrogen, alkyl, substituted alkyl, aryl, substituted aryl, heteroaryl, substituted heteroaryl, heterocyclic and substituted heterocyclic, provided that when X is —SO— or —SO²— , then R^{a6} is not hydrogen, and R^{a7} is selected from the group consisting of hydrogen, alkyl, aryl, or R^{a2}, R^{a3} together with the carbon atom pendent thereto, form an aryl, substituted aryl, heteroaryl, or substituted heteroaryl;
or a pharmaceutically acceptable salt, ester, or prodrug thereof;
with the proviso that at least one of the following occurs:
1) R^{a1} is fluoro, bromo, iodo, alkyl, substituted alkyl, alkoxy, aminoacyl, substituted alkoxy, aryl, substituted aryl, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl; or
2) R^{a2} is substituted alkyl, aryl, substituted aryl, heteroaryl, substituted heteroaryl, fluoro, bromo, iodo, cyano, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a2} is substituted alkyl such a substituent does not include trifluoromethyl;
   b) —XR^{a6} is not alkoxy; and
   c) when —XR^{a6} is substituted alkoxy such a substituent does not include benzyl or benzyl substituted by a substituent selected from the group consisting of (C₁-C₅) alkyl and (C₁-C₅) alkoxy or does not include a fluoroalkoxy substituent of the formula:
   —O—[CH₂]ₓ—C_{f}H_{(2f+1-g)}F_{g}
where x is zero or one; f is an integer of from 1 to 5; and
g is an integer of from 1 to (2f+1).

In embodiments, a compound is (1-methyl-4-hydroxy-7-phenoxy-isoquinoline-3-carbonyl)-amino-acetic acid (Compound 4), or a pharmaceutically acceptable salt thereof. Compound 4, also referred to as roxadustat, or FG-4592, has the following structure:

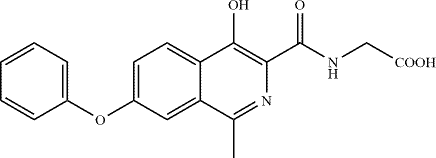 [SMILES: Cc1nc(C(=O)NCC(=O)O)c(O)c2ccc(Oc3ccccc3)cc12]

### Formula (V): Daprodustat and Related Compounds

Still other compounds that can be useful in the methods described herein include those described in U.S. Pat. No. 8,324,208, which is incorporated by reference in its entirety. In embodiments, a compound, or a pharmaceutically acceptable salt thereof, is described in any of claims 1-16 of U.S. Pat. No. 8,324,208.

In embodiments, suitable compounds include a compound having a structure according to Formula (V),

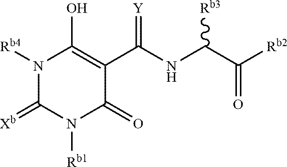

or a pharmaceutically acceptable salt thereof, wherein
R^{b1} and R^{b4} are each independently selected from the group consisting of hydrogen, —NR^{b5}R^{b6}, C₁-C₁₀ alkyl, C₂-C₁₀ alkenyl, C₂-C₁₀ alkynyl, C₃-C₈ cycloalkyl, C₃-C₈ cycloalkyl-C₁-C₁₀ alkyl, C₅-C₈ cycloalkenyl, C₅-C₈ cycloalkenyl- C₁-C₁₀ alkyl, C₃-C₈ heterocycloalkyl, C₃-C₈ heterocycloalkyl-C₁-C₁₀ alkyl, aryl, aryl- C₁-C₁₀ alkyl, heteroaryl and heteroaryl- C₁-C₁₀ alkyl;
R^{b2} is —NR^{b7}R^{b8} or —OR^{b9};
R^{b3} is H or C₁-C₄ alkyl;
where R^{b5} and R^{b6} are each independently selected from the group consisting of hydrogen, C₁-C₁₀ alkyl, C₃-C₈ cycloalkyl, C₃-C₈ cycloalkyl-C₁-C₁₀ alkyl, C₃-C₈ heterocycloalkyl, C₃-C₈ heterocycloalkyl-C₁-C₁₀ alkyl, aryl, aryl- C₁-C₁₀ alkyl, heteroaryl and heteroaryl- C₁-C₁₀ alkyl, — C(O)C₁-C₄ alkyl, —C(O)C₃—C₆ cycloalkyl, —C(O)C₃—C₆ heterocycloalkyl, —C(O)aryl, — C(O)heteroaryl and —S(O)₂ C₁-C₄ alkyl, or, when R^{b5} and R^{b6} are attached to the same nitrogen, R^{b5} and R^{b6} taken together with the nitrogen to which they are attached forma 5- or 6- or 7-membered saturated ring optionally containing one other heteroatom selected from oxygen, nitrogen and sulphur,
R^{b7} and R^{b8} are each independently selected from the group consisting of hydrogen, C₁-C₁₀ alkyl, C₂-C₁₀ alkenyl, C₃-C₈ cycloalkyl, C₃-C₈ heterocycloalkyl, aryl, and heteroaryl, and
R^{b9} is H or a cation, or C₁-C₁₀ alkyl which is unsubstituted or substituted with one or more substituents independently selected from the group consisting of C₃-C₆ cycloalkyl, heterocycloalkyl, aryl and heteroaryl;
X^{b} is O or S; and
Y is O or S;
where any carbon or heteroatom of R^{b1}, R^{b2}, R^{b3}, R^{b4}, R^{b5}, R^{b6}, R^{b7}, R^{b8}, R^{b9} is unsubstituted or is substituted with one or more substituents independently selected from C₁-C₆ alkyl, C₁-C₆ haloalkyl, halogen, — OR^{b10}, —NR^{b5}R^{b6}, oxo, cyano, nitro, —C(O)R^{b10}, —C(O)OR^{b10} —SR^{b10}, — S(O)R^{b10}, — S(O)₂R^{b10}, — CONR^{b5}R^{b6}, — N(R^{b5})C(O)R^{b10}, —N(R^{b5})C(O)OR^{b10}, —OC(O)NR^{b5}R^{b6}', — N(R^{b5})C(O)NR^{b5}R^{b6}, — SO₂NR^{b5}R^{b6}, — N(R^{b5})SO₂R^{b10}, C₂-C₁₀ alkenyl, C₂-C₁₀ alkynyl, C₃-C₆ cycloalkyl, C₃-C₆ heterocycloalkyl, aryl, C₁-C₆ alkyl-aryl, heteroaryl and C₁-C₆ alkyl-heteroaryl, wherein R^{b5} and R^{b6} are the same as defined above and R^{b10} is selected from hydrogen, C₁-C₁₀ alkyl, C₂-C₁₀ alkenyl, C₂-C₁₀ alkynyl. —C(O)C₁C₄ alkyl, -C(O)aryl, -C(O)heteroaryl, —C(O)C₃—C₆ cycloalkyl, —C(O)C₃—C₆ heterocycloalkyl, —S(O)₂C₁—C₄ alkyl, C₃-C₈ cycloalkyl, C₃-C₈ heterocycloalkyl, C₆-C₁₄ aryl, aryl-C₁-C₁₀ alkyl, heteroaryl and heteroaryl-C₁-C₁₀ alkyl;

In embodiments, X^{b} is O. In embodiments, Y is O.

In embodiments, R^{b1} and R^{b4} are each independently selected from the group consisting of hydrogen, —NR^{b5}R^{b6}, C₁-C₁₀ alkyl, C₂-C₁₀ alkenyl, C₂-C₁₀ alkynyl, C₃-C₈ cycloalkyl, C₃-C₈ cycloalkyl-C₁-C₁₀ alkyl, C₅-C₈ cycloalkenyl, C₅-C₈ cycloalkenyl- C₁-C₁₀ alkyl, C₃-C₈ heterocycloalkyl, C₃-C₈ heterocycloalkyl-C₁-C₁₀ alkyl, aryl, aryl- C₁-C₁₀ alkyl, heteroaryl and heteroaryl- C₁-C₁₀ alkyl. In embodiments, R^{b1} is C₃-C₈ cycloalkyl. In embodiments, R^{b4} is C₃-C₈ cycloalkyl.

In embodiments, R^{b2} is —OR^{b9}, wherein R^{b9} is H or a cation. In embodiments, R^{b2} is OH.

In embodiments, R^{b3} is H.

In embodiments, a compound is N-(1,3-Dicyclohexyl-6-hydroxy-2,4-dioxo-1,2,3,4-tetrahydro-5-pyrimidinyl)carbonylglycine (Compound 5), or a pharmaceutically acceptable salt thereof. Compound 5, also referred to as daprodustat, or GSK-12788363, has the following structure:

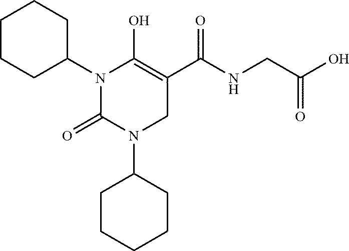 [SMILES: O=C(O)CNC(=O)C1=C(O)N(C2CCCCC2)C(=O)N(C2CCCCC2)C1]

### Formula (VI): Molidustat and Related Compounds

Still other compounds that can be useful in the methods described herein include those described in U.S. Pat. No. 8,389,520, which is incorporated by reference in its entirety. In embodiments, a compound, or a pharmaceutically acceptable salt thereof, is described in any of claims 1-10 of U.S. Pat. No. 8,389,520.

In embodiments, suitable compounds include a compound having a structure according to Formula (VI),

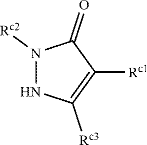

or a pharmaceutically acceptable salt thereof, wherein
R^{c1} represents a heteroaryl group of the formula

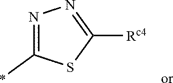

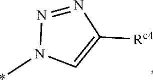

, wherein
* denotes the linkage point with the dihydropyrazolone ring and
R^{c4} denotes hydrogen, fluorine, chlorine, bromine, cyano, C₁-C₄ alkyl, trifluoromethyl, hydroxymethyl, C₁-C₄ alkoxy, trifluoromethoxy, hydroxycarbonyl or C₁-C₄ alkoxycarbonyl;
R^{c2} represents a heteroaryl group of the formula

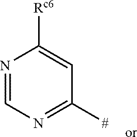

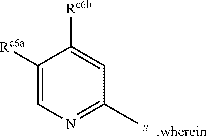

# denotes the linkage point with the dihydropyrazolone ring and
R^{c6}, R^{c6a} and R^{c6b} are identical or different and independently of one another denote hydrogen or a substituent chosen from the group consisting of fluorine, chlorine, bromine, cyano, C₁-C₆ alkyl, trifluoromethyl, hydroxyl, C₁-C₆ alkoxy, trifluoromethoxy, amino, mono-C₁-C₄ alkylamino, di-C₁-C₄ alkylamino, hydroxycarbonyl, C₁-C₄ alkoxycarbonyl, 4-to 6-membered heterocycloalkyl, phenyl and 5-or 6-membered heteroaryl, wherein
C₁-C₆ alkyl in its turn can be substituted by hydroxyl, C₁-C₄ alkoxy or amino and
4-to 6-membered heterocycloalkyl, phenyl and 5-or 6-membered heteroaryl in their turn can in each case be substituted once or twice in an identical or different manner by fluorine, chlorine, bromine, cyano, C₁-C₄ alkyl, trifluoromethyl, hydroxyl, C₁-C₄ alkoxy, trifluoromethoxy, oxo, amino, mono-C₁-C₄ alkylamino, di-C₁-C₄ alkylamino, hydroxycarbonyl or C₁-C₄ alkoxycarbonyl, and
R^{c3} represents hydrogen, or a salt thereof.

In embodiments, R^{c1} is a heteroaryl group of the formula

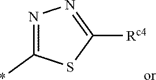

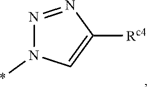

wherein * denotes the linkage point with the dihydropyrazolone ring, and wherein R^{c4} is hydrogen, fluorine, chlorine, bromine, cyano, C₁-C₄ alkyl, trifluoromethyl, hydroxymethyl, C₁-C₄ alkoxy, trifluoromethoxy, hydroxycarbonyl or C₁-C₄ alkoxycarbonyl.

In embodiments, R^{c2} is a heteroaryl group of the formula

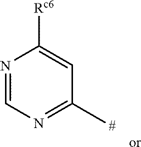

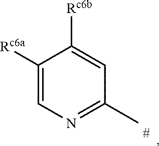

wherein # denotes the linkage point with the dihydropyrazolone ring and wherein R^{c6}, R^{c6a} and R^{c6b} are identical or different and independently of one another denote hydrogen or a substituent chosen from the group consisting of fluorine, chlorine, bromine, cyano, C₁-C₆ alkyl, trifluoromethyl, hydroxyl, C₁-C₆ alkoxy, trifluoromethoxy, amino, mono-C₁-C₄ alkylamino, di-C₁-C₄ alkylamino, hydroxycarbonyl, C₁-C₄ alkoxycarbonyl, 4-to 6-membered heterocycloalkyl, phenyl and 5-or 6-membered heteroaryl. In embodiments, a C₁-C₆ alkyl is unsubstituted C₁-C₆ alkyl. In embodiments, a C₁-C₆ alkyl is substituted C₁-C₆ alkyl. In embodiments, a C₁-C₆ alkyl is substituted with hydroxyl, C₁-C₄ alkoxy or amino. In embodiments, a 4-to 6-membered heterocycloalkyl is unsubstituted. In embodiments, a 4-to 6-membered heterocycloalkyl is substituted. In embodiments, a 4-to 6-membered heterocycloalkyl is substituted with once or twice in an identical or different manner by fluorine, chlorine, bromine, cyano, C₁-C₄ alkyl, trifluoromethyl, hydroxyl, C₁-C₄ alkoxy, trifluoromethoxy, oxo, amino, mono-C₁-C₄ alkylamino, di-C₁-C₄ alkylamino, hydroxycarbonyl or C₁-C₄ alkoxycarbonyl. In embodiments, a phenyl is unsubstituted. In embodiments, a phenyl is substituted. In embodiments, a phenyl is substituted with once or twice in an identical or different manner by fluorine, chlorine, bromine, cyano, C₁-C₄ alkyl, trifluoromethyl, hydroxyl, C₁-C₄ alkoxy, trifluoromethoxy, oxo, amino, mono-C₁-C₄ alkylamino, di-C₁-C₄ alkylamino, hydroxycarbonyl or C₁-C₄ alkoxycarbonyl. In embodiments, a 5-or 6-membered heteroaryl is unsubstituted. In embodiments, a 5-or 6-membered heteroaryl is substituted. In embodiments, a 5-or 6-membered heteroaryl is substituted with once or twice in an identical or different manner by fluorine, chlorine, bromine, cyano, C₁-C₄ alkyl, trifluoromethyl, hydroxyl, C₁-C₄ alkoxy, trifluoromethoxy, oxo, amino, mono-C₁-C₄ alkylamino, di-C₁-C₄ alkylamino, hydroxycarbonyl or C₁-C₄ alkoxycarbonyl.

In embodiments, R^{c3} is hydrogen.

In embodiments, a compound 2-(6-Morpholin-4-ylpyrimidin-4-yl)-4-(1H-1,2,3-triazol-1- yl)-1,2-dihydro-3H-pyrazol-3-one (Compound 6), or a pharmaceutically acceptable salt thereof. Compound 6, also referred to as molidustat, or BAY 85-3934, has the following structure:

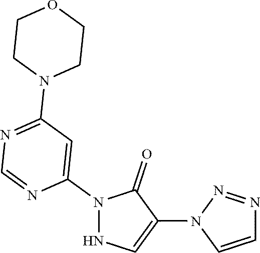 [SMILES: O=c1c(-n2ccnn2)c[nH]n1-c1cc(N2CCOCC2)ncn1]

### Methods of Treatment

### HIF Stabilization and Hypoxia Signaling in Human Diseases

Described herein are new therapeutic methods comprising administering compounds that can stabilize HIF and inhibit HIF prolyl hydroxylase (HIF-PH). In particular, methods described herein can be useful for treating a disease or condition in patients in need thereof, such as a patient having a viral infection (e.g., a respiratory or pulmonary viral infection such as COVID-19 or another coronavirus infection). For example, methods described herein can be useful for treating or preventing acute respiratory distress syndrome (ARDS) in a patient in need thereof (e.g., a patient with COVID-19 such as a hospitalized patient with COVID-19), where the method comprises administering a compound that can stabilize HIF and inhibit HIF prolyl hydroxylase (HIF-PH) (e.g., vadadustat).

Methods described herein can also be useful for treating/preventing organ injury (e.g. organ injury that occurs concurrently, or as induced by or associated with an infection). In embodiments, an organ injury is an acute organ injury. For example, methods described herein can be useful for treating or preventing acute lung injury (ALI), acute respiratory distress syndrome (ARDS), cardiovascular injury, injury to the liver, injury to the pancreas, neurological injury, kidney diseases, and/or multi-organ failure.

Suitable HIF prolyl hydroxylase inhibitors or HIF-alpha stabilizers include (but are not limited to) those described herein, including compounds according to any one of Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

Exemplary methods are described herein.

### Viral Infections

Methods described herein can be useful for treating patients having a viral infection, including pulmonary viral infections and/or respiratory viral infections. For example, methods described herein can be beneficial for patients having a coronavirus infection (e.g., COVID-19). Methods described herein also can be beneficial in treating or preventing organ injury in patients having a viral infection (e.g., a pulmonary or respiratory viral infection). For example, methods described herein can be useful for treating and/or preventing injury to the lung (e.g., acute lung injury (ALI) and/or acute respiratory distress syndrome (ARDS)), liver, and/or heart (e.g., cardiovascular injury), and kidney diseases. Further, methods described herein are useful for treating or preventing injury to multiple organs (e.g., multi-organ failure and/or septic shock).

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

Exemplary viral infections are described herein.

### Viruses

### Coronaviruses

In embodiments, methods described herein can treat: a coronavirus infection (e.g., a pulmonary coronavirus infection and/or a respiratory coronavirus infection), one or more conditions (e.g., organ injury) induced by or associated with a coronavirus infection (e.g., a pulmonary coronavirus infection and/or a respiratory coronavirus infection), and/or one of more conditions in a patient having a coronavirus infection (e.g., a pulmonary coronavirus infection and/or a respiratory coronavirus infection).

Coronaviruses (CoVs) are a family of single-stranded RNA viruses. CoVs have been demonstrated to cross species barriers and can cause illness in human ranging in degrees of severity. Coronavirus infection, as used herein, means an infection, including a patient being infected, with any coronavirus virus including HCoV-NL63, HCoV-OC43, HCoV-229E, HCoV-HKU1, SARS-CoV (Severe Acute Respiratory Syndrome-Coronavirus), CoV MERS (Middle East Respiratory Syndrome virus, previously called “EMC”), and SARS-CoV-2 (Severe Acute Respiratory Syndrome Coronavirus 2, the virus that causes COVID-19 disease).

Coronaviruses cause approximately 10-15% of all upper and lower respiratory tract infections. They account for significant hospitalizations of children under 18 years of age, the elderly and immunocompromised individuals. According to a number of international studies 5-10% of the acute respiratory diseases are caused by HCoV-NL63. These numbers are probably an underestimation since during diagnostic screening for respiratory viruses tests for HCoV’s are frequently not included. Another aspect HCoV-NL63 infection is the co-infection with other human coronaviruses, influenza A, respiratory syncytial virus (RSV), parainfluenza virus human metapneumovirus. In children they are associated with acute respiratory tract illness, pneumonia and Croup leading in many cases to hospitalization.

In particular, methods described herein can be beneficial to a patient infected by a coronavirus that is SARS-CoV, MERS-CoV, or SARS-CoV-2 (e.g., having a respiratory viral infection and/or a pulmonary viral infection). In embodiments, a patient is infected by SARS-CoV-2: patients with COVID-19 therefore can benefit from methods described herein for treating conditions induced by or associated with the viral infection or conditions present in the patient.

### Influenza Viruses

In embodiments, methods described herein can treat: an influenza virus infection (e.g., a pulmonary influenza virus infection and/or a respiratory influenza virus infection), one or more conditions (e.g., organ injury) induced by or associated with an influenza virus infection (e.g., a pulmonary influenza virus infection and/or a respiratory influenza virus infection), and/or one of more conditions in a patient having an influenza virus infection (e.g., a pulmonary influenza virus infection and/or a respiratory influenza virus infection).

Influenza viruses are divided into three types, type A, B and C, based upon differences in internal antigenic proteins. The Influenza A virus may be further classified into various subtypes according to the different HA and NA viral proteins displayed on the surface of the virus. Each subtype of virus can mutate into a variety of strains with differing pathogenic profiles. Currently, there are 16 known HA antigen subtypes (H1 to H16) and 9 known NA antigen subtypes (N1 to N9). Influenza A viruses can infect humans, birds, pigs, horses, and other animals. A subset of Influenza A virus subtypes, including but not limited to, H1N1, H2N2, H3N2, H5N1, H7N7, H1N2, H9N2, H7N2, H7N3, and H10N7 subtypes, have been confirmed to infect humans. All combinations of the 16 HA and 9 NA subtypes have been identified in avian species. In addition, Influenza B virus and Influenza C virus can also infect humans.

Due to viral recombination, prior immunity to one strain does not necessarily confer protection to the next. Upon infection, a new virus replicates unchecked, while the host mounts a highly inflammatory primary immune response. An influenza infection produces an acute set of symptoms including headache, cough, sore throat, rhinitis, fever and general malaise. In severe cases or situations involving pre-existing pulmonary or cardiovascular disease, hospitalization is required. Pneumonia due to direct viral infection or due to secondary bacterial or viral invasion is the most frequent complication.

The outcome of influenza infection is dependent on both the virus and the host. The genetic makeup of the HA and NA genes confers virulence. For example, introduction of HA and NA genes from pandemic H5N1 strains to a relatively mild virus transforms the virus into a highly virulent strain in mice. During replication, Influenza virus utilizes host protein production machinery and as a result, causes death of the infected cell (cytopathology). Such respiratory epithelial cell destruction produces an array of signals initiating an inflammatory reaction (cytokine cascade) that promotes the recruitment of inflammatory cells (e.g., neutrophils and CD4+/CD8+ T cells) to the delicate surface of the lung, leading to consolidation of air spaces and a decline in arterial oxygen saturation. In eliminating the virus, the host response causes further respiratory cell death, and the responding inflammatory cells (e.g., T cells) produce an additional battery of inflammatory mediators (e.g., TNFα and IFNγ), which in excess lead to a cytokine storm, causing capillary leak and resulting in pulmonary edema and leukocyte transudation into the airspaces, thereby initiating the acute respiratory distress syndrome (ARDS). More chronic symptoms of disease, such as cachexia, fever and appetite suppression, are directly linked to the concentration of systemic mediators/cytokines that accumulate. Therefore, the whole cascade is initiated by virus-induced cytopathology, but mortality is ultimately determined by the magnitude of the inflammation that results from the immune response. Both the viral-induced cytopathology and the host inflammatory response can predispose the infected subject to secondary bacterial infection, further increasing morbidity and mortality.

### Respiratory Syncytial Virus (RSV)

In embodiments, methods described herein can treat: a respiratory syncytial virus infection (e.g., a pulmonary RSV infection and/or a respiratory RSV infection), one or more conditions (e.g., organ injury) induced by or associated with a RSV infection (e.g., a pulmonary RSV infection and/or a respiratory RSV infection), and/or one of more conditions in a patient having a RSV infection (e.g., a pulmonary RSV infection and/or a respiratory RSV infection).

Respiratory syncytial virus, or RSV, is a member of Paramyxoviridae family of nonsegmented, negative-sense, single-stranded RNA genome viruses. It is a common respiratory virus that usually causes mild, cold-like symptoms. But it can cause serious lung infections. In fact, it is a leading cause of lower respiratory tract infections in infants, young children, and the elderly or immunocompromised.

### Adenovirus

In embodiments, methods described herein can treat: an adenovirus infection (e.g., a pulmonary adenovirus infection and/or a respiratory adenovirus infection), one or more conditions (e.g., organ injury) induced by or associated with an adenovirus infection (e.g., a pulmonary adenovirus infection and/or a respiratory adenovirus infection), and/or one of more conditions in a patient having an adenovirus infection (e.g., a pulmonary adenovirus infection and/or a respiratory adenovirus infection).

Adenovirus is a one of the common viruses that cause a range of illness, including cold-like symptoms, fever, sore throat, bronchitis, pneumonia, diarrhea, and pink eye (conjunctivitis). Adenovirus, a member of the Adenoviridae family, is a nonenveloped (without an outer lipid bilayer) virus with an icosahedral nucleocapsid containing a double stranded DNA genome. The classification of Adenoviridae can be complex. In humans, there are 57 accepted human adenovirus types (HAdV-1 to 57) in seven species (Human adenovirus A to G), and different types/serotypes are associated with different conditions.

### Human Parainfluenza Virus (HPIV)

In embodiments, methods described herein can treat: a human parainfluenza virus infection (e.g., a pulmonary human parainfluenza virus infection and/or a respiratory human parainfluenza virus infection), one or more conditions (e.g., organ injury) induced by or associated with a human parainfluenza virus infection (e.g., a pulmonary human parainfluenza virus infection and/or a respiratory human parainfluenza virus infection), and/or one of more conditions in a patient having a human parainfluenza virus infection (e.g., a pulmonary human parainfluenza virus infection and/or a respiratory human parainfluenza virus infection).

Human parainfluenza virus (HPIV). There are four viruses in this group (HPIV-1 to 4), each one of which causes different symptoms and illnesses. All forms of HPIV can cause an infection in either the upper or lower respiratory area of a person’s body. HPIV-1 is the leading cause of croup in children, which is a respiratory illness that manifests as swelling near the vocal cords and in other parts of the upper respiratory system. HPIV-1 is responsible for outbreaks of croup in the autumn.

### Parainfluenza Viruses (PIV)

In embodiments, methods described herein can treat: a parainfluenza virus infection (e.g., a pulmonary parainfluenza virus infection and/or a respiratory parainfluenza virus infection), one or more conditions (e.g., organ injury) induced by or associated with a parainfluenza virus infection (e.g., a pulmonary parainfluenza virus infection and/or a respiratory parainfluenza virus infection), and/or one of more conditions in a patient having a parainfluenza virus infection (e.g., a pulmonary parainfluenza virus infection and/or a respiratory parainfluenza virus infection).

Parainfluenza viruses (PIV) are paramyxoviruses of the order Mononegavirales, the family Paramyxoviridae, and the subfamily Paramyxovirinae. Human PIVs (HPIVs) are currently divided into 5 serotypes—HPIV-1, HPIV-2, HPIV-3, HPIV-4a, and HPIV-4b—in 2 different genera: Respirovirus (HPIV-1 and HPIV-3) and Rubulavirus (HPIV-2 and HPIV-4). Each one causes different symptoms and illnesses. HPIVs primarily affect young children, in whom the pathogenic spectrum includes upper and lower respiratory tract infections. They are responsible for 30%-40% of all acute respiratory tract infections in infants and children. These conditions include common cold with fever, laryngotracheobronchitis (croup), bronchiolitis, and pneumonia. HPIVs are also a cause of community-acquired respiratory tract infections of variable severity in adults.

### Enterovirus

In embodiments, methods described herein can treat: an enterovirus infection (e.g., a pulmonary enterovirus infection and/or a respiratory enterovirus infection), one or more conditions (e.g., organ injury) induced by or associated with an enterovirus infection (e.g., a pulmonary enterovirus infection and/or a respiratory enterovirus infection), and/or one of more conditions in a patient having an enterovirus infection (e.g., a pulmonary enterovirus infection and/or a respiratory enterovirus infection).

Enterovirus is a member of the picornavirus family, a large and diverse group of small RNA viruses characterized by a single positive-strand genomic RNA. The enterovirus genus includes fifteen species (enterovirus A to L and rhinovirus A to C). Enteroviruses affect millions of people worldwide each year and are often found in the respiratory secretions (e.g., saliva, sputum, or nasal mucus) and stool of an infected person. Historically, poliomyelitis was the most significant disease caused by an enterovirus, namely poliovirus. There are 81 non-polio and 3 polio enteroviruses that can cause disease in humans. Of the 81 non-polio types, there are 22 Coxsackie A viruses, 6 Coxsackie B viruses, 28 echoviruses, and 25 other enteroviruses.

### Rhinoviruses

In embodiments, methods described herein can treat: a rhinovirus infection (e.g., a pulmonary rhinovirus infection and/or a respiratory rhinovirus infection), one or more conditions (e.g., organ injury) induced by or associated with a rhinovirus infection (e.g., a pulmonary rhinovirus infection and/or a respiratory rhinovirus infection), and/or one of more conditions in a patient having a rhinovirus infection (e.g., a pulmonary rhinovirus infection and/or a respiratory rhinovirus infection).

Rhinoviruses is one of the most common causative agent in humans for the common cold. Rhinoviruses belong to the Enterovirus genus in the family Picornaviridae. There are three species of rhinovirus (A, B, and C) which include approximately 160 recognized types of human rhinovirus that differ according to the virus’s surface proteins. The species of rhinovirus have been associated with the severity of the infection caused by the virus. For example, rhinovirus C appear to cause more severe infections that rhinoviruses A and B. The mode of transmission for rhinoviruses are predominately via aerosols of respiratory droplets and from virus-contaminated surfaces.

### Respiratory Viral Infection and Pulmonary Viral Infection

Methods described herein can be useful for treating viral infections such as respiratory viral infections and/or pulmonary viral infections (e.g., COVID-19 related pneumonia). For example, methods described herein can be useful in treating conditions induced by or associated with a respiratory viral infection and/or a pulmonary viral infection. Further, methods described herein can be useful in treating a condition in a patient having a respiratory viral infection and/or a pulmonary viral infection.

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

Respiratory viral infection commonly affect the upper (sinuses, nasal passages, pharynx, and larynx) or lower (trachea, the bronchi and bronchioles, and the alveoli, which make up the lungs) respiratory tract. It may cause coughing, sneezing, runny noses, sore throats or fever. Respiratory means something that affects the lungs and airways (breathing passages). Viral means something that is caused by a virus. Viruses that cause VRIs include respiratory syncytial viruses (RSV), influenza viruses, parainfluenza viruses, adenoviruses and rhinoviruses. Rhinoviruses are the viruses that cause the common cold. Although respiratory infections can be classified by the causative virus, they are generally classified clinically according to syndrome, such as the common cold, bronchiolitis, croup, pneumonia. While specific pathogens commonly cause characteristic clinical manifestations (e.g., rhinovirus typically causes the common cold, respiratory syncytial virus (RSV) typically causes bronchiolitis), each can cause many of the viral respiratory syndromes. Severity of viral respiratory illness varies widely. Morbidity may result directly from viral infection or may be indirect, due to exacerbation underlying medical conditions.

In embodiments, the invention relates to a method for treating a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is the SARS-CoV, MERS-CoV, or SARS-CoV-2. In embodiments, a coronavirus is SARS-CoV-2.

In embodiments, a respiratory viral infection is lower respiratory infection. In embodiments, a respiratory viral infection is upper respiratory infection. In embodiments, a respiratory viral infection is pulmonary viral infection.

In embodiments, a respiratory viral infection is COVID-19.

In embodiments, a respiratory viral infection induces a lung disease. In embodiments, a lung disease is acute lung injury (ALI). In embodiments, a lung disease is bronchitis. In embodiments, a lung disease is pneumonia. In embodiments, a lung disease is pulmonary fibrosis. In embodiments, a lung disease is asthma. In embodiments, a lung disease is acute respiratory distress syndrome (ARDS).

In embodiments, the invention relates to a method for treating a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is the SARS-CoV, MERS-CoV, or SARS-CoV-2. In embodiments, a coronavirus is SARS-CoV-2.

In embodiments, a pulmonary viral infection is COVID-19.

In embodiments, a pulmonary viral infection induces a lung disease. In embodiments, a lung disease is acute lung injury (ALI). In embodiments, a lung disease is bronchitis. In embodiments, a lung disease is pneumonia. In embodiments, a lung disease is pulmonary fibrosis. In embodiments, a lung disease is asthma. In embodiments, a lung disease is acute respiratory distress syndrome (ARDS).

In embodiments, a patient with lung disease develops pulmonary hypertension. In embodiments, a patient with lung disease develops multiorgan failure. In embodiments, the multiorgan failure comprises heart failure, liver failure, lung failure kidney failure, and gastrointestinal (GI) system failure. In embodiments, a patient with lung disease develops heart failure. In embodiments, a patient with lung disease develops liver failure. In embodiments, a patient with lung disease develops lung failure. In embodiments, a patient with lung disease develops kidney failure. In embodiments, a patient with lung disease develops gastrointestinal (GI) system failure.

In embodiments, a respiratory and/or pulmonary infection induces heart diseases (e.g. cardiac dysfunction and/or adverse cardiac events, or hypotension), liver diseases (e.g. liver failure), kidney diseases, and/or other diseases or conditions (e.g. septic shock, sepsis, cytokine release syndrome, disseminated intravascular coagulation, pancreas injury, neurological disorder, and pulmonary barotrauma).

Exemplary methods of administration (including exemplary dosage amounts) are provided herein.

### COVID-19 and Coronavirus Infection

Methods described herein can be useful for treating viral infections such as coronavirus infections. For example, methods described herein can be useful in treating or preventing conditions induced by or associated with a coronavirus infection, such as COVID-19. Further, methods described herein can be useful in treating or preventing a condition in a patient having a coronavirus infection (e.g. a patient infected by SARS-CoV-2).

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

Coronaviruses such as the human CoV isolates 229E and OC43 cause mild and self-limiting infections of the respiratory tract such as the common cold. Novel isolates HCoV-NL63 and HCoV-HKU1 have also been associated with common cold.

Some novel emerging coronaviruses have resulted in serious global outbreaks such as SARS in 2003, MERS in 2012 and COVID-19 in 2019.

In embodiments, the invention relates to a method for treating a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus is the SARS-CoV, MERS-CoV, or SARS-CoV-2. In embodiments, a coronavirus is SARS-CoV-2.

In embodiments, a coronavirus infection induces a lung disease. In embodiments, a lung disease is acute lung injury (ALI). In embodiments, a lung disease is bronchitis. In embodiments, a lung disease is pneumonia. In embodiments, a lung disease is pulmonary fibrosis. In embodiments, a lung disease is asthma. In embodiments, a lung disease is acute respiratory distress syndrome (ARDS).

In embodiments, a coronavirus infection induces heart diseases (e.g. cardiac dysfunction and/or adverse cardiac events, or hypotension), liver diseases (e.g. liver failure), kidney diseases, and/or other diseases or conditions (e.g. septic shock, sepsis, cytokine release syndrome, disseminated intravascular coagulation, pancreas injury, neurological disorder, and pulmonary barotrauma).

In embodiments, a coronavirus infection is COVID-19.

In embodiments, the invention relates to a method for treating or preventing COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a patient with COVID-19 develops multiorgan failure. In embodiments, multiorgan failure comprises heart failure, liver failure, lung failure kidney failure, and gastrointestinal (GI) system failure.

In embodiments, COVID-19 induces lung diseases (e.g. acute lung injury, acute respiratory distress syndrome, lung inflammation, and pneumonia). In embodiments, the invention relates to a method for treating or preventing of lung diseases (e.g. acute lung injury, acute respiratory distress syndrome, lung inflammation, and pneumonia) induced by and/or related to COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, COVID-19 induces heart diseases (e.g. cardiac dysfunction and/or adverse cardiac events, or hypotension). In embodiments, the invention relates to a method for treating or preventing of heart diseases (e.g. cardiac dysfunction and/or adverse cardiac events, or hypotension) induced by and/or related to COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, COVID-19 induces liver diseases (e.g. liver failure). In embodiments, the invention relates to a method for treating or preventing of liver diseases (e.g. liver failure) induced by and/or related to COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, COVID-19 induces kidney diseases. In embodiments, the invention relates to a method for treating or preventing of kidney diseases induced by and/or related to COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, COVID-19 induces other diseases or conditions (e.g. septic shock, sepsis, cytokine release syndrome, disseminated intravascular coagulation, pancreas injury, neurological disorder, and pulmonary barotrauma). In embodiments, the invention relates to a method for treating or preventing of other diseases or conditions (e.g. septic shock, sepsis, cytokine release syndrome, disseminated intravascular coagulation, pancreas injury, neurological disorder, multiorgan disorder/dysfunction and pulmonary barotrauma) induced by and/or related to COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing COVID-19 related acute lung injury (ALI) in a patient in need thereof, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing COVID-19 related acute respiratory distress syndrome (ARDS) in a patient in need thereof, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing acute respiratory distress syndrome (ARDS) in a hospitalized patient with COVID-19, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing COVID-19 related pneumonia in a patient in need thereof, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing COVID-19 related organ failure in a patient in need thereof, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a patient has been diagnosed with COVID-19. In embodiments, a patient is suspected to have COVID-19. In embodiments, a patient is hospitalized.

Exemplary methods of administration (including exemplary dosage amounts) are provided herein.

### HIF Stabilization and Lung Injury (Including Lung Diseases)

Methods described herein can be useful for treating or prevention lung diseases such as acute lung injury (ALI), acute respiratory distress syndrome (ARDS), lung inflammation and/or pneumonia in a patient in need thereof. For example, methods described herein can be useful in treating or preventing lung diseases in a patient having a respiratory viral infection, a pulmonary viral infection, and/or a coronavirus infection (e.g., COVID-19 related pneumonia). In embodiments, the lung disease is induced by or associated with a viral infection. Further, methods described herein can also be useful in treating or preventing lung diseases in a patient without viral infection.

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

Hypoxia itself may contribute to lung injury and thereby perpetuate the injury by impairing endothelial cells, attracting white blood cells, and promoting inflammation. Stabilizing HIF has been shown to be protective during ALI. HIF has been suggested to promote repair and spreading of alveolar type 2 (ATII) cells after ALI. Alveolar type I (ATI) cells are extremely thin squamous cells that line the alveoli and are involved in gas exchange. The ATII cells synthesize, store, and release surfactant to lower surface tension to increase lung compliance and are also involved in repair following alveolar injury - the ATII cells can proliferate and differentiate into ATI cells. A third cell type, the alveolar macrophage, are phagocytic cells that ingest foreign particles. As with cardiac disease, long term upregulation of HIF may have deleterious effects on chronic lung conditions such as pulmonary hypertension, pulmonary fibrosis, and chronic obstructive pulmonary disease, although this seems less relevant to the acute application envisioned here.

### Acute Lung Injury (ALI) and Acute Respiratory Distress Syndrome (ARDS)

In embodiments, described herein are methods for treating or prevention lung diseases such as acute lung injury (ALI) and/or acute respiratory distress syndrome (ARDS). For example, methods described herein can be useful in treating or preventing lung diseases in a patient having a viral infection (e.g. respiratory viral infection, pulmonary viral infection, and/or coronavirus infection), sepsis, pneumonia, aspiration, trauma, pancreatitis, blood transfusion, and/or smoke or toxic gas inhalation. In embodiments, methods described herein can be useful for treating or preventing acute respiratory distress syndrome (ARDS) in a patient in need thereof (e.g., a patient with COVID-19 such as a hospitalized patient with COVID-19), where the method comprises administering a compound that can stabilize HIF and inhibit HIF prolyl hydroxylase (HIF-PH) (e.g., vadadustat).

Acute lung injury (ALI) and acute respiratory distress syndrome (ARDS) represent a spectrum of acute respiratory failure with diffuse, bilateral lung injury and severe hypoxemia caused by non-cardiogenic pulmonary edema. Failure may be initiated by pulmonary or extrapulmonary insults (e.g. pneumonia, sepsis, trauma, aspiration) that increase alveolar epithelial endothelial permeability, flood alveoli, and reduce lung compliance.

The severity of hypoxaemia distinguishes ALI from ARDS: when hypoxaemia is severe (partial arterial pressure of oxygen (PaO2)/fractional concentration of oxygen in inspired air (FIO2)<200 mmHg or 26.7 kPa), the disorder is termed ARDS, whereas less severe abnormalities of gas exchange (PaO2/FIO2<300 mmHg or 40 kPa) are termed ALI.

In embodiments, acute lung injury (ALI) or acute respiratory distress syndrome (ARDS) is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

In embodiments, the invention relates to a method for treating or preventing a lung disease that is acute lung injury (ALI) in a patient in need thereof, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, an acute lung injury (ALI) is induced by or associated with a viral infection, sepsis, pneumonia, aspiration, trauma, pancreatitis, blood transfusion, and/or smoke or toxic gas inhalation. In embodiments, an acute lung injury (ALI) is induced by or associated with a viral infection (e.g. respiratory viral infection, pulmonary viral infection, and/or coronavirus infection). In embodiments, an acute lung injury (ALI) is induced by or associated with a ventilator. In embodiment, an acute lung injury (ALI) is induced by or associated with COVID-19.

In embodiments, a patient has a viral infection, sepsis, pneumonia, aspiration, trauma, pancreatitis, blood transfusion, and/or smoke or toxic gas inhalation. In embodiments, a patient is mechanically ventilated. In embodiments, a patient has a viral infection.

In embodiments, the invention relates to a method for treating or preventing a lung disease that is acute lung injury (ALI) in a patient in need thereof, wherein said ALI is induced by or associated with a ventilator, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is acute lung injury (ALI) in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is acute respiratory distress syndrome (ARDS) in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is acute lung injury (ALI) in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is acute respiratory distress syndrome (ARDS) in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is acute lung injury (ALI) in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is acute respiratory distress syndrome (ARDS) in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing a lung disease that is acute lung injury (ALI) in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is acute respiratory distress syndrome (ARDS) in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is acute lung injury (ALI) in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is acute respiratory distress syndrome (ARDS) in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is acute lung injury (ALI) in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is acute respiratory distress syndrome (ARDS) in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing a lung disease that is acute lung injury (ALI) in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is acute respiratory distress syndrome (ARDS) in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is acute lung injury (ALI) in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is acute respiratory distress syndrome (ARDS) in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is acute lung injury (ALI) in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is acute respiratory distress syndrome (ARDS) in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, an acute lung injury (ALI) and/or acute respiratory distress syndrome (ARDS) is induced by or associated with a viral infection (e.g. respiratory viral infection, pulmonary viral infection, and/or coronavirus infection). In embodiment, an acute lung injury (ALI) and/or acute respiratory distress syndrome (ARDS) is induced by or associated with COVID-19. In embodiment, an acute lung injury (ALI) and/or acute respiratory distress syndrome (ARDS) is induced by or associated with COVID-19.

In embodiments, a patient with acute lung injury (ALI) develops acute respiratory distress syndrome (ARDS). In embodiments, a patient with ALI or ARDS develops pulmonary hypertension. In embodiments, a patient with ALI or ARDS develops multiorgan failure. In embodiments, multiorgan failure comprises heart failure, liver failure, lung failure kidney failure, and gastrointestinal (GI) system failure. In embodiments, a patient with lung disease develops heart failure. In embodiments, a patient with lung disease develops liver failure. In embodiments, a patient with lung disease develops lung failure. In embodiments, a patient with lung disease develops kidney failure. In embodiments, a patient with lung disease develops gastrointestinal (GI) system failure.

In embodiments, an acute lung injury (ALI) or acute respiratory distress syndrome (ARDS) induces organ dysfunction, and the invention relates to a method for treating or preventing of organ dysfunction induced by or associated with acute lung injury (ALI) or acute respiratory distress syndrome (ARDS), comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

In embodiments, the invention relates to a method for treating or preventing acute respiratory distress syndrome (ARDS) in a hospitalized patient with COVID-19, comprising administering to the patient a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

### Lung Inflammation and Pneumonia

Pneumonia is an inflammatory condition of the lung affecting primarily the small air sacs known as alveoli. It is usually caused by a virus, bacteria, fungi or other germs. It happens when an infection causes alveoli to fill with fluid or pus. That can make it hard for you to breathe in enough oxygen to reach your bloodstream. Symptoms typically include some combination of productive or dry cough, chest pain, fever and difficulty breathing, and the severity of the condition is variable.

In embodiments, “pulmonary inflammation” and “lung inflammation” can be used interchangeably.

In embodiments, the invention relates to a method for treating or preventing a lung disease that is lung inflammation in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is pneumonia in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is lung inflammation in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is pneumonia in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is lung inflammation in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is pneumonia in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing a lung disease that is lung inflammation in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is pneumonia in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is lung inflammation in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is pneumonia in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is lung inflammation in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is pneumonia in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing a lung disease that is lung inflammation in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating or preventing a lung disease that is pneumonia in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is lung inflammation in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to a lung disease that is pneumonia in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is lung inflammation in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of a lung disease that is pneumonia in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by SARS-CoV-2.

In embodiments, a coronavirus infection is COVID-19.

In embodiments, a lung inflammation and/or pneumonia is induced by or associated with a viral infection (e.g. respiratory viral infection, pulmonary viral infection, and/or coronavirus infection). In embodiment, a lung inflammation and/or pneumonia is induced by or associated with COVID-19.

In embodiments, methods described herein are useful in treating or preventing pulmonary inflammation (or lung inflammation), including inflammation associated with or caused by viral infections (e.g., respiratory viral infections, pulmonary viral infections, or coronavirus infections) or pneumonia, in a patient in need thereof.

In embodiments, methods described herein are useful in reducing mortality and morbidity related to pulmonary inflammation (or lung inflammation), including inflammation associated with or caused by viral infections (e.g., respiratory viral infections, pulmonary viral infections, or coronavirus infections) or pneumonia, in a patient in need thereof.

In embodiments, methods described herein are useful in reducing incidence, severity, or risk of pulmonary inflammation (or lung inflammation), including inflammation associated with or caused by viral infections (e.g., respiratory viral infections, pulmonary viral infections, or coronavirus infections) or pneumonia, in a patient in need thereof.

In embodiments, a patient with lung inflammation or pneumonia develops pulmonary hypertension. In embodiments, a patient with lung inflammation or pneumonia develops multiorgan failure. In embodiments, the multiorgan failure comprises heart failure, liver failure, lung failure kidney failure, and gastrointestinal (GI) system failure. In embodiments, a patient with lung inflammation or pneumonia develops heart failure. In embodiments, a patient with lung inflammation or pneumonia develops liver failure. In embodiments, a patient with lung inflammation or pneumonia develops lung failure. In embodiments, a patient with lung inflammation or pneumonia develops kidney failure. In embodiments, a patient with lung inflammation or pneumonia develops gastrointestinal (GI) system failure.

In embodiments, lung inflammation or pneumonia induces organ dysfunction, and the invention relates to a method for treating or preventing of organ dysfunction induced by or associated with lung inflammation or pneumonia, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a lung injury or lung disease described herein such as lung inflammation is characterized by body weight change (e.g, body weight loss). In embodiments, a method described herein can result in increased body weight change (e.g., body weight loss). In embodiments, a method described herein can result in decreased body weight change (e.g., body weight loss).

In embodiments, a lung injury or lung disease described herein such as lung inflammation is characterized by cell differentials (e.g, cell differentials in neutrophils, lymphocytes, macrophages, mast cells, eosinophils, and basophils). In embodiments, a method described herein can result in changes in cell differentials.

In embodiments, a lung injury or lung disease described herein such as lung inflammation is characterized by total nucleated cell counts. In embodiments, a method described herein can result in changes (e.g., decreases) in total nucleated cell counts.

In embodiments, a lung injury or lung disease described herein such as lung inflammation is characterized by BAL (bronchoalveolar lavage) cytokine (e.g, IL-6 (Interleukin-6) or TNFα (Tumor Necrosis Factor-α)) concentrations. In embodiments, a method described herein can result in changes (e.g., increases) in BAL cytokine (e.g, IL-6 (Interleukin-6) or TNFα(Tumor Necrosis Factor-α)) concentrations.

In embodiments, a lung injury or lung disease described herein such as lung inflammation is characterized by peribronchiolar lymphoid inflammation width. In embodiments, a method described herein can result in changes (e.g., decreases) in peribronchiolar lymphoid inflammation width.

In embodiments, lung inflammation described herein is characterized by lung histopathology scores (e.g., neutrophil, interstitial inflammation score, peribronchiolar/perivascular edema, and airway epithelial damage). In embodiments, a method described herein can result in improvements in histopathology scores (e.g., improvements in neutrophil, interstitial inflammation score, peribronchiolar/perivascular edema, and/or airway epithelial damage). In embodiments, a method described herein can result in changes (e.g., decreases) in neutrophil and interstitial inflammation scores.

In embodiments, lung inflammation described herein is characterized by lung summed inflammation scores. In embodiments, a method described herein can result in changes (e.g., decreases) in lung summed inflammation scores.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### HIF Stabilization and Cardiovascular Injury and Diseases (Including Heart Diseases)

Methods described herein can be useful for treating or prevention cardiovascular conditions and diseases, including heart diseases such as cardiac dysfunction and/or hypotension. For example, methods described herein can be useful in treating or preventing cardiovascular conditions or diseases (such as heart diseases) in a patient having a respiratory viral infection, a pulmonary viral infection, and/or a coronavirus infection (e.g., COVID-19 related pneumonia). In embodiments, the cardiovascular disease or condition (e.g., heart disease) is induced by or associated with a viral infection (e.g., COVID-19 related pneumonia). In embodiments, the cardiovascular disease or condition (e.g., heart disease) occurs independently of a viral infection.

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

HIF stabilization increased nitric oxide (NO) synthase and heme oxygenase 1 (HO1) both of which decrease inflammatory responses. HIF is a central component of ischemic preconditioning (IPC) in the heart. IPC involves administering short, repetitive bouts of ischemia and reperfusion to increase the hearts ability to tolerate a subsequent bout of prolonged ischemia. HIF also contributed to ischemic postconditioning, repeated bouts of ischemia following a bout of prolonged ischemia, which is known to decrease myocardial infarct size. Long term upregulation of HIF may have deleterious effects on the heart in conditions such as congestive heart failure, although this seems less relevant to the acute application envisioned here.

### Cardiac Dysfunction

In embodiments, the invention relates to a method for treating or preventing cardiac dysfunction and/or adverse cardiac events in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to cardiac dysfunction and/or adverse cardiac events in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of cardiac dysfunction and/or adverse cardiac events in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing cardiac dysfunction and/or adverse cardiac events in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to cardiac dysfunction and/or adverse cardiac events in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of cardiac dysfunction and/or adverse cardiac events in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing cardiac dysfunction and/or adverse cardiac events in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to cardiac dysfunction and/or adverse cardiac events in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of cardiac dysfunction and/or adverse cardiac events in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a cardiac dysfunction and/or adverse cardiac events is induced by or associated with a viral infection (e.g. respiratory viral infection, pulmonary viral infection, and/or coronavirus infection). In embodiment, a cardiac dysfunction and/or adverse cardiac events is induced by or associated with COVID-19.

In embodiments, a patient with cardiac dysfunction and/or adverse cardiac events develops multiorgan failure. In embodiments, the multiorgan failure comprises heart failure, liver failure, lung failure kidney failure, and gastrointestinal (GI) system failure. In embodiments, a patient with cardiac dysfunction and/or adverse cardiac events develops heart failure. In embodiments, a patient with cardiac dysfunction and/or adverse cardiac events develops liver failure. In embodiments, a patient with cardiac dysfunction and/or adverse cardiac events develops lung failure. In embodiments, a patient with cardiac dysfunction and/or adverse cardiac events develops kidney failure. In embodiments, a patient with cardiac dysfunction and/or adverse cardiac events develops gastrointestinal (GI) system failure.

In embodiments, a cardiac dysfunction and/or adverse cardiac events induces organ dysfunction, and the invention relates to a method for treating or preventing of organ dysfunction induced by or associated with cardiac dysfunction and/or adverse cardiac events, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Hypotension

Hypotension is low blood pressure. In healthy people, low blood pressure without any symptoms is not usually a concern and does not need to be treated. But low blood pressure can be a sign of an underlying problem, where it may cause inadequate blood flow to the heart, brain, and other vital organs.

In embodiments, the invention relates to a method for treating or preventing hypotension in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to hypotension in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of hypotension in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing hypotension in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to hypotension in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of hypotension in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing hypotension in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to hypotension in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of hypotension in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a hypotension is induced by or associated with a viral infection (e.g. respiratory viral infection, pulmonary viral infection, and/or coronavirus infection). In embodiment, a hypotension is induced by or associated with COVID-19.

In embodiments, a patient with hypotension develops multiorgan failure. In embodiments, the multiorgan failure comprises heart failure, liver failure, lung failure, kidney failure, and gastrointestinal (GI) system failure. In embodiments, a patient with hypotension develops heart failure. In embodiments, a patient with hypotension develops liver failure. In embodiments, a patient with hypotension develops lung failure. In embodiments, a patient with hypotension develops kidney failure. In embodiments, a patient with hypotension develops gastrointestinal (GI) system failure.

In embodiments, a hypotension induces organ dysfunction, and the invention relates to a method for treating or preventing of organ dysfunction induced by or associated with hypotension, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### HIF Stabilization and Liver Injury (Including Liver Diseases)

Methods described herein can be useful for treating or preventing liver injury in a patient, including liver diseases such as liver failure. For example, methods described herein can be useful in treating or preventing liver injury in a patient having a respiratory viral infection, a pulmonary viral infection, and/or a coronavirus infection (e.g., COVID-19 related pneumonia). In embodiments, the liver injury is induced by or associated with a viral infection (e.g., COVID-19 related pneumonia). In embodiments, the liver injury occurs independently of a viral infection.

In embodiments, liver injury is characterized by abnormal levels of any abnormality in aspartate transaminase (AST), alanine transaminase (ALT), gamma-glutamyltransferase (GGT), and/or alkaline phosphatase (ALP).

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

HIF has been shown to be beneficial in ischemia reperfusion (IR) injury of the liver. Pharmacological induction of HIF ameliorates the IR liver injuries by upregulating HIF1A-dependent antioxidant genes and reducing mitochondrial damage. A transgenic mouse model lacking PHD1 (therefore activating HIF) displays tolerance to acute hypoxia and protection against IR liver injury. PHD deletion also promoted liver regeneration in a hepatectomy model. As with the heart and lung, HIF upregulation has negative long term consequence in conditions such as liver fibrosis.

### Liver Failure

Liver failure is the loss of liver function characterized by increased serum alanine and aspartate aminotransferase levels, prothrombin time, and serum bilirubin. Common causes for this condition are acetaminophen (APAP) overdose, excessive alcohol consumption, acute viral hepatitis, and IRI. Treatment is focused on reversing APAP overdose by N-acetylcysteine or providing supportive management until the liver function returns back to normal.

In embodiments, the invention relates to a method for treating or preventing liver failure in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to liver failure in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of liver failure in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing liver failure in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to liver failure in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of liver failure in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing liver failure in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to liver failure in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of liver failure in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a liver failure is induced by or associated with a viral infection (e.g. respiratory viral infection, pulmonary viral infection, and/or coronavirus infection). In embodiment, a liver failure is induced by or associated with COVID-19.

In embodiments, a patient with liver failure develops multiorgan failure. In embodiments, the multiorgan failure comprises heart failure, liver failure, lung failure kidney failure, and gastrointestinal (GI) system failure. In embodiments, a patient with liver failure develops heart failure. In embodiments, a patient with liver failure develops lung failure. In embodiments, a patient with liver failure develops kidney failure. In embodiments, a patient with liver failure develops gastrointestinal (GI) system failure.

In embodiments, a liver failure induces organ dysfunction, and the invention relates to a method for treating or preventing of organ dysfunction induced by or associated with liver failure, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### HIF Stabilization and Kidney Diseases

Methods described herein can be useful for treating or preventing kidney diseases. For example, methods described herein can be useful in treating or preventing kidney diseases in a patient having a respiratory viral infection, a pulmonary viral infection, and/or a coronavirus infection (e.g., COVID-19 related pneumonia). In embodiments, the kidney disease is induced by or associated with a viral infection (e.g., COVID-19 related pneumonia). In embodiments, the kidney disease occurs independently of a viral infection.

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

In embodiments, the invention relates to a method for treating or preventing kidney diseases in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to kidney diseases in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of kidney diseases in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing kidney diseases in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to kidney diseases in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of kidney diseases in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing kidney diseases in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to kidney diseases in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of kidney diseases in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a kidney disease is induced by a viral infection (e.g. respiratory viral infection, pulmonary viral infection, and/or coronavirus infection). In embodiment, a kidney disease is induced by COVID-19.

In embodiments, a patient with kidney disease develops multiorgan failure. In embodiments, the multiorgan failure comprises heart failure, liver failure, lung failure kidney failure, and gastrointestinal (GI) system failure. In embodiments, a patient with kidney disease develops heart failure. In embodiments, a patient with kidney disease develops liver failure. In embodiments, a patient with kidney disease develops lung failure. In embodiments, a patient with kidney disease develops kidney failure. In embodiments, a patient with kidney disease develops gastrointestinal (GI) system failure.

In embodiments, a kidney disease induces organ dysfunction, and the invention relates to a method for treating or preventing of organ dysfunction induced by or associated with kidney diseases, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### HIF Stabilization for Treating Other Diseases

Methods described herein can be useful for treating or prevention other diseases, including those associated with viral infection. For example, methods described herein can be useful in treating or preventing diseases (e.g. septic shock, sepsis, cytokine release syndrome, disseminated intravascular coagulation, pancreas injury, neurological disorder, multiorgan disorder/dysfunction and pulmonary barotrauma) in a patient having a respiratory viral infection, a pulmonary viral infection, and/or a coronavirus infection (e.g., COVID-19 related pneumonia). In embodiments, the diseases described herein is induced by or associated with a viral infection (e.g., COVID-19 related pneumonia). In embodiments, diseases described herein occur independently of a viral infection.

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

### Septic Shock

Septic shock, namely, infection throughout the body, is a potentially fatal medical condition that occurs when sepsis, which is organ injury or damage in response to infection, leads to dangerously low blood pressure and abnormalities in cellular metabolism.

In embodiments, the invention relates to a method for treating or preventing septic shock in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to septic shock in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of septic shock in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing septic shock in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to septic shock in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of septic shock in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing septic shock in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to septic shock in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of septic shock in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a septic shock is induced by or associated with a viral infection (e.g. respiratory viral infection, pulmonary viral infection, and/or coronavirus infection). In embodiment, a septic shock is induced by or associated with COVID-19.

In embodiments, a patient with septic shock develops multiorgan failure. In embodiments, the multiorgan failure comprises heart failure, liver failure, lung failure kidney failure, and gastrointestinal (GI) system failure. In embodiments, a patient with septic shock develops heart failure. In embodiments, a patient with septic shock develops liver failure. In embodiments, a patient with septic shock develops lung failure. In embodiments, a patient with septic shock develops kidney failure. In embodiments, a patient with septic shock develops gastrointestinal (GI) system failure.

In embodiments, a septic shock induces organ dysfunction, and the invention relates to a method for treating or preventing of organ dysfunction induced by or associated with septic shock, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Sepsis

Sepsis is a potentially life-threatening condition caused by the body’s response to an infection. The body normally releases chemicals into the bloodstream to fight an infection. Sepsis occurs when the body’s response to these chemicals is out of balance, triggering changes that can damage multiple organ systems.

In embodiments, the invention relates to a method for treating or preventing sepsis in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to sepsis in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of sepsis in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing sepsis in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to sepsis in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of sepsis in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing sepsis in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to sepsis in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of sepsis in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a sepsis is induced by or associated with a viral infection (e.g. respiratory viral infection, pulmonary viral infection, and/or coronavirus infection). In embodiment, a sepsis is induced by or associated with COVID-19.

In embodiments, a patient with sepsis develops multiorgan failure. In embodiments, the multiorgan failure comprises heart failure, liver failure, lung failure kidney failure, and gastrointestinal (GI) system failure. In embodiments, a patient with sepsis develops heart failure. In embodiments, a patient with sepsis develops liver failure. In embodiments, a patient with sepsis develops lung failure. In embodiments, a patient with sepsis develops kidney failure. In embodiments, a patient with sepsis develops gastrointestinal (GI) system failure.

In embodiments, a sepsis induces organ dysfunction, and the invention relates to a method for treating or preventing of organ dysfunction induced by or associated with sepsis, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Cytokine Release Syndrome (Cytokine Storm)

Cytokine release syndrome (CRS) is an acute systemic inflammatory syndrome characterized by fever and multiple organ dysfunction that is associated with chimeric antigen receptor (CAR)-T cell therapy, therapeutic antibodies, and haploidentical allogeneic transplantation.

In embodiments, the invention relates to a method for treating or preventing cytokine release syndrome in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to cytokine release syndrome in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of cytokine release syndrome in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing cytokine release syndrome in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to cytokine release syndrome in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of cytokine release syndrome in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing cytokine release syndrome in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to cytokine release syndrome in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of cytokine release syndrome in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a cytokine release syndrome is induced by or associated with a viral infection (e.g. respiratory viral infection, pulmonary viral infection, and/or coronavirus infection). In embodiment, a cytokine release syndrome is induced by or associated with COVID-19.

In embodiments, a patient with cytokine release syndrome develops multiorgan failure. In embodiments, the multiorgan failure comprises heart failure, liver failure, lung failure kidney failure, and gastrointestinal (GI) system failure. In embodiments, a patient with cytokine release syndrome develops heart failure. In embodiments, a patient with cytokine release syndrome develops liver failure. In embodiments, a patient with cytokine release syndrome develops lung failure. In embodiments, a patient with cytokine release syndrome develops kidney failure. In embodiments, a patient with cytokine release syndrome develops gastrointestinal (GI) system failure.

In embodiments, a cytokine release syndrome induces organ dysfunction, and the invention relates to a method for treating or preventing of organ dysfunction induced by or associated with cytokine release syndrome, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Disseminated Intravascular Coagulation

Disseminated intravascular coagulation is a condition in which small blood clots develop throughout the bloodstream, blocking small blood vessels. The increased clotting depletes the platelets and clotting factors needed to control bleeding, causing excessive bleeding.

In embodiments, the invention relates to a method for treating or preventing disseminated intravascular coagulation in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to disseminated intravascular coagulation in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of disseminated intravascular coagulation in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing disseminated intravascular coagulation in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to disseminated intravascular coagulation in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of disseminated intravascular coagulation in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing disseminated intravascular coagulation in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to disseminated intravascular coagulation in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of disseminated intravascular coagulation in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a disseminated intravascular coagulation is induced by or associated with a viral infection (e.g. respiratory viral infection, pulmonary viral infection, and/or coronavirus infection). In embodiment, a disseminated intravascular coagulation is induced by or associated with COVID-19.

In embodiments, a patient with disseminated intravascular coagulation develops multiorgan failure. In embodiments, the multiorgan failure comprises heart failure, liver failure, lung failure kidney failure, and gastrointestinal (GI) system failure. In embodiments, a patient with disseminated intravascular coagulation develops heart failure. In embodiments, a patient with disseminated intravascular coagulation develops liver failure. In embodiments, a patient with disseminated intravascular coagulation develops lung failure. In embodiments, a patient with disseminated intravascular coagulation develops kidney failure. In embodiments, a patient with disseminated intravascular coagulation develops gastrointestinal (GI) system failure.

In embodiments, a disseminated intravascular coagulation induces organ dysfunction, and the invention relates to a method for treating or preventing of organ dysfunction induced by or associated with disseminated intravascular coagulation, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Pancreas Injury

A pancreas injury is some form of trauma sustained by the pancreas. The injury can be sustained through either blunt forces, such as a motor vehicle accident, or penetrative forces, such as that of a gunshot wound. The pancreas is one of the least commonly injured organs in abdominal trauma.

In embodiments, the invention relates to a method for treating or preventing pancreas injury in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to pancreas injury in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of pancreas injury in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing pancreas injury in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to pancreas injury in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of pancreas injury in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing pancreas injury in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to pancreas injury in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of pancreas injury in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a pancreas injury is induced by or associated with a viral infection (e.g. respiratory viral infection, pulmonary viral infection, and/or coronavirus infection). In embodiment, a pancreas injury is induced by or associated with COVID-19.

In embodiments, a patient with pancreas injury develops multiorgan failure. In embodiments, the multiorgan failure comprises heart failure, liver failure, lung failure kidney failure, and gastrointestinal (GI) system failure. In embodiments, a patient with pancreas injury develops heart failure. In embodiments, a patient with pancreas injury develops liver failure. In embodiments, a patient with pancreas injury develops lung failure. In embodiments, a patient with pancreas injury develops kidney failure. In embodiments, a patient with pancreas injury develops gastrointestinal (GI) system failure.

In embodiments, a pancreas injury induces organ dysfunction, and the invention relates to a method for treating or preventing of organ dysfunction induced by or associated with pancreas injury, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Neurological Disorder

A neurological disorder is any disorder of the nervous system. Structural, biochemical or electrical abnormalities in the brain, spinal cord or other nerves can result in a range of symptoms.

In embodiments, the invention relates to a method for treating or preventing neurological disorder in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to neurological disorder in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of neurological disorder in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing neurological disorder in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to neurological disorder in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of neurological disorder in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing neurological disorder in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to neurological disorder in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of neurological disorder in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a neurological disorder is induced by or associated with a viral infection (e.g. respiratory viral infection, pulmonary viral infection, and/or coronavirus infection). In embodiment, a neurological disorder is induced by or associated with COVID-19.

In embodiments, a patient with neurological disorder develops multiorgan failure. In embodiments, the multiorgan failure comprises heart failure, liver failure, lung failure kidney failure, and gastrointestinal (GI) system failure. In embodiments, a patient with neurological disorder develops heart failure. In embodiments, a patient with neurological disorder develops liver failure. In embodiments, a patient with neurological disorder develops lung failure. In embodiments, a patient with neurological disorder develops kidney failure. In embodiments, a patient with neurological disorder develops gastrointestinal (GI) system failure.

In embodiments, a neurological disorder induces organ dysfunction, and the invention relates to a method for treating or preventing of organ dysfunction induced by or associated with neurological disorder, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Pulmonary Barotrauma

Pulmonary barotrauma is physical damage to body tissues caused by a difference in pressure between a gas space inside, or in contact with, the body, and the surrounding gas or fluid. Pulmonary barotrauma is damage to the lung from rapid or excessive pressure changes, as may occur when a patient is on a ventilator and is subjected to high airway pressure.

In embodiments, the invention relates to a method for treating or preventing pulmonary barotrauma in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to pulmonary barotrauma in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of pulmonary barotrauma in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing pulmonary barotrauma in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to pulmonary barotrauma in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of pulmonary barotrauma in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing pulmonary barotrauma in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to pulmonary barotrauma in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of pulmonary barotrauma in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a pulmonary barotrauma is induced by or associated with a viral infection (e.g. respiratory viral infection, pulmonary viral infection, and/or coronavirus infection). In embodiment, a pulmonary barotrauma is induced by or associated with COVID-19.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Multi-Organ Failure

Multiple organ failure (MOF) is a syndrome that represents a complicated and dynamic pathophysiologic pathway leading to organ functional derangement and eventual death. Severe hemorrhagic shock begins an inflammatory cascade that cannot be reversed in some patients despite adequate resuscitation.

In embodiments, the invention relates to a method for treating or preventing multiple organ failure in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to multiple organ failure in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of multiple organ failure in a patient having a respiratory viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a respiratory viral infection is induced by or associated with a virus that could lead to viral pneumonia. In embodiments, a respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a respiratory viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a respiratory viral infection is COVID-19.

In embodiments, the invention relates to a method for treating or preventing multiple organ failure in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to multiple organ failure in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of multiple organ failure in a patient having a pulmonary viral infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a pulmonary viral infection is induced by or associated with a virus that could lead to viral pneumonia. In embodiments, a pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a pulmonary viral infection is induced by or associated with a coronavirus. In embodiments, a coronavirus is SARS-CoV, MERS-CoV, or SARS-CoV-2. In embodiments, a coronavirus is SARS-CoV-2.

In embodiments, a pulmonary viral infection is COVID-19.

In embodiments, the invention relates to a method for treating or preventing multiple organ failure in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing mortality and morbidity related to multiple organ failure in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing incidence, severity, or risk of multiple organ failure in a patient having a coronavirus infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a coronavirus infection is induced by or associated with a virus that could lead to viral pneumonia. In embodiments, a coronavirus infection is induced by or associated with SARS-CoV, MERS-CoV, or SARS-CoV-2. In embodiments, a coronavirus infection is induced by or associated with SARS-CoV-2.

In embodiments, a coronavirus infection is COVID-19.

In embodiments, a multiple organ failure is induced by or associated with a viral infection (e.g. respiratory viral infection, pulmonary viral infection, and/or coronavirus infection). In embodiment, a multiple organ failure is induced by or associated with COVID-19.

In embodiments, a viral infection is a coronavirus infection. In embodiments, the coronavirus is SARS-CoV, MERS-CoV, or SARS-CoV-2. In embodiments, the coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Induced Organ Dysfunction

Methods described herein can be useful for treating or prevention organ dysfunction induced by or associated with a condition or disease as described anywhere herein (e.g. COVID-19, acute lung injury, acute respiratory distress syndrome, lung inflammation, or pneumonia).

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

In embodiments, the invention relates to a method for treating and/or preventing organ dysfunction induced by or associated with a disease or condition as described herein, such as COVID-19, lung diseases (e.g. acute lung injury, acute respiratory distress syndrome, lung inflammation, and/or pneumonia), heart diseases (e.g. cardiac dysfunction and/or hypotension), kidney diseases, and other diseases (e.g. septic shock, and/or cytokine release syndrome), or any combination thereof. In embodiments, the invention relates to a method for treating and/or preventing organ dysfunction induced by or associated with a disease or condition as described herein, or any combination thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with acute lung injury (ALI), comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with acute respiratory distress syndrome (ARDS), comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

0733] In embodiments, the invention relates to a method for treating and/or preventing organ dysfunction induced by or associated with lung inflammation, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with pneumonia, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

0735] In embodiments, the invention relates to a method for treating and/or preventing organ dysfunction induced by or associated with cardiac dysfunction and/or adverse cardiac events, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with hypotension, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with liver failure, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with kidney diseases, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with septic shock, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with sepsis, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with cytokine release syndrome (cytokine storm), comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

0742] In embodiments, the invention relates to a method for treating and/or preventing organ dysfunction induced by or associated with disseminated intravascular coagulation, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

0743] In embodiments, the invention relates to a method for treating and/or preventing organ dysfunction induced by or associated with pancreas injury, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with neurological disorder, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating and/or preventing organ dysfunction induced by or associated with COVID-19, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a patient has a disease or condition as described as anywhere herein. In embodiments, a patient has COVID-19. In embodiments, a patient has been diagnosed with COVID-19. In embodiments, a patient is suspected to have COVID-19. In embodiments, a patient is hospitalized.

### Additional Therapeutic Uses

Methods described herein can be useful for in a patient I need thereof (e.g. a patient having a respiratory viral infection, a pulmonary viral infection, and/or a coronavirus infection).

In embodiments, the invention relates to a method for improving lung volumes in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for improving pulmonary compliance in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for promoting fibroproliferative repair in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for regulating innate and adaptive immunity in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for promoting regulatory T-cells, lymphocytes and B cells in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for treating/preventing bronchial inflammation in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for reducing cytokine production in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the invention relates to a method for improving dyspnea in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

0756] In embodiments, the invention relates to a method for improving arterial PaO2 in a patient in need thereof, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a patient has a viral infection. In embodiments, a viral infection is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a viral infection is induced by or associated with a coronavirus. In embodiments, a coronavirus is SARS-CoV, MERS-CoV, or SARS-CoV-2. In embodiments, a coronavirus is SARS-CoV-2. In embodiments, a patient has COVID-19. In embodiments, a patient has been diagnosed with COVID-19. In embodiments, a patient is suspected to have COVID-19. In embodiments, a patient is hospitalized.

### Combination Therapy

In embodiments, a HIF prolyl hydroxylase inhibitor or a HIF-alpha stabilizer may be administered in combination with another medicament or another therapy.

In embodiments, the other medicament or therapy is an antiviral agent.

In embodiments, a HIF prolyl hydroxylase inhibitor or a HIF-alpha stabilizer, is administered in combination with an antiviral agent. Various antiviral agents are known in the art. In some embodiments, an antiviral agent is a neuraminidase inhibitor and/or an adamantine derivative. Non-limiting examples of suitable neuraminidase inhibitor include Tamiflu® and Relenza®. Non-limiting examples of adamantine derivatives include amantadine and rimantadine. In some embodiments, a HIF prolyl hydroxylase inhibitor or a HIF-alpha stabilizer, is administered in combination with one or more of the following antiviral drugs: Remdesivir, Chloroquine, Lopinavir and ritonavir, APN01, Favilavir, Baricitinib, Abacavir, Acyclovir, Adefovir, Amantadine, Ampligen, Amprenavir, Arbidol, Atazanavir, Atripla, Balavir, Baloxavir marboxil, Biktarvy, Cidofovir, Combivir, Darunavir, Delavirdine, Descovy, Didanosine, Docosanol, Dolutegravir, Ecoliever, Edoxudine, Efavirenz, Emtricitabine, Enfuvirtide, Entecavir, Famciclovir, Fomivirsen, Fosamprenavir, Foscarnet, Fosfonet, Ibacitabine, Idoxuridine, Imiquimod, Imunovir, Indinavir, Inosine, Integrase inhibitor, Interferon type 1, Interferon type II, Interferon type III, Interferon, Lamivudine, Lopinavir, Loviride, Maraviroc, Methisazone, Moroxydine, Nelfinavir, Nevirapine, Nexavir, Nitazoxanide, Norvir, Nucleoside analogues, Oseltamivir, Peginterferon alfa-2a, Penciclovir, Peramivir, Pleconaril, Podophyllotixin, Protease inhibitor, Pyramidine, Raltegravir, Reverse transcriptase inhibitor, Ribavirin, Rimantadine, Ritonavir, Saquinavir, Sofosbuvir, Stavudine, Telaprevir, Tenofovir alafenamide, Tenofovir disoproxil, Tenofovir, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine.

In some embodiments, administration of the HIF prolyl hydroxylase inhibitor or HIF-alpha stabilizer occurs simultaneously with the antiviral drug.

In some embodiments, administration of the HIF prolyl hydroxylase inhibitor or HIF-alpha stabilizer occurs after administration of the antiviral drug. For example, administration of the HIF prolyl hydroxylase inhibitor or HIF-alpha stabilizer occurs about 6 hours, 12 hours, 24 hours, 2 days, 3 days, 4 days, or 5 days after administration of the antiviral drug. In some embodiments, administration of the HIF prolyl hydroxylase inhibitor or HIF-alpha stabilizer occurs more than 5 days after administration of the antiviral drug.

In some embodiments, administration of the HIF prolyl hydroxylase inhibitor or HIF-alpha stabilizer occurs before administration of the antiviral drug. For example, administration of the antiviral drug occurs about 6 hours, 12 hours, 24 hours, 2 days, 3 days, 4 days, or 5 days after administration of the HIF prolyl hydroxylase inhibitor or HIF-alpha stabilizer. In some embodiments, administration of the antiviral drug occurs more than 5 days after administration of the HIF prolyl hydroxylase inhibitor or HIF-alpha stabilizer.

In embodiments, the other medicament or therapy is oxygen therapy. In embodiments, an oxygen therapy comprises mechanical ventilation. In embodiments, a patient is receiving mechanical ventilation.

In some embodiments, administration of the HIF prolyl hydroxylase inhibitor or HIF-alpha stabilizer occurs during the use of oxygen therapy.

In some embodiments, administration of the HIF prolyl hydroxylase inhibitor or HIF-alpha stabilizer occurs after the use of oxygen therapy. For example, administration of the HIF prolyl hydroxylase inhibitor or HIF-alpha stabilizer occurs about 1 day, 2 days, 3 days, 4 days, or 5 days after the use of oxygen therapy. In some embodiments, administration of the HIF prolyl hydroxylase inhibitor or HIF-alpha stabilizer occurs more than 5 days after the use of oxygen therapy.

In some embodiments, administration of the HIF prolyl hydroxylase inhibitor or HIF-alpha stabilizer occurs before the use of oxygen therapy. For example, the use of oxygen therapy occurs about 1 day, 2 days, 3 days, 4 days, or 5 days after administration of the HIF prolyl hydroxylase inhibitor or HIF-alpha stabilizer. In some embodiments, the use of oxygen therapy occurs more than 5 days after administration of the HIF prolyl hydroxylase inhibitor or HIF-alpha stabilizer.

### Patient Populations and Severity of the Disease

### Patient Populations

In embodiments, the invention relates to methods for treating and/or preventing a disease or condition as described anywhere herein. In particular, methods described herein can be useful for treating acute respiratory and pulmonary inflammatory conditions such as in patients having a viral infection (e.g., patients having a pulmonary viral infection (e.g., a coronavirus infection such as COVID-19) or a respiratory viral infection (e.g., a coronavirus infection such as COVID-19) and/or preventing organ injury that occurs concurrently or as a result of an infection, comprising administering to a patient in need thereof a compound that is a HIF prolyl hydroxylase inhibitor or a HIF-alpha stabilizer. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. An embodiments, a compound is vadadustat.

In embodiments, a HIF prolyl hydroxylase inhibitor or a HIF-alpha stabilizer is administered to a patient in need thereof.

In embodiments, a patient has a viral infection. In embodiments, a viral infection is a respiratory viral infection (e.g. upper respiratory infection and/or lower respiratory infection.). In embodiments, a viral infection is a pulmonary viral infection. In embodiments, a viral infection (e.g. respiratory viral infection and/or pulmonary viral infection) is induced by or associated with a virus that could lead to viral pneumonia. In embodiments, a viral infection (e.g. respiratory viral infection and/or pulmonary viral infection) is induced by or associated with a coronavirus, an influenza A, an influenza B, a Rhinovirus, a respiratory syncytial virus (RSV), an adenovirus, a human parainfluenza virus (HPIV) or an enterovirus. In embodiments, a viral infection (e.g. respiratory viral infection and/or pulmonary viral infection) is induced by or associated with a coronavirus. In embodiments, a coronavirus is the SARS-CoV, MERS-CoV, or SARS-CoV-2. In embodiments, a coronavirus is SARS-CoV-2. In embodiments, a patient has disease or condition induced by or associated with a viral infection. In embodiments, a patient has a disease or condition described anywhere herein.

In embodiments, a patient has one or more clinically-recognized symptoms associated with the respiratory viral infection. In embodiments, a patient has one or more clinically-recognized symptoms associated with the pulmonary viral infection. In embodiments, a patient has one or more clinically-recognized symptoms associated with the coronavirus infection. In embodiments, a patient has one or more clinically-recognized symptoms associated the diseases or conditions described herein, such as lung diseases (e.g. acute lung injury, acute respiratory distress syndrome, lung inflammation and/or pneumonia), heart diseases (e.g. cardiac dysfunction, and/or hypotension), liver diseases (e.g. liver failure), kidney diseases, and other diseases (e.g. septic shock, and/or cytokine release syndrome). In embodiments, a patient has one or more clinically-recognized symptoms associated with COVID-19. Examples of symptoms associated with COVID-19 include fever, dry cough, fatigue, coughing up sputum from the lungs, bone or joint pain, sore throat, headache, chills, nausea or vomiting, stuffy nose, pressure or pain in the chest, shortness of breath, sudden confusion, digestive issues, conjunctivitis, bluish face or lips, and loss of smell or taste.

In embodiments, a patient exhibits one or more symptoms of hypoxia. In embodiments, the one or more symptoms include changes in the color of the skin, confusion, cough, fast heart rate, rapid breathing, shortness of breath, sweating, and/or wheezing. In embodiments, a patient has decreased oxygen saturation of hemoglobin relative to normal levels. In embodiments, a patient an oxygen saturation of hemoglobin that is 94% or less.

In embodiments, a patient has an elevated level of an inflammation marker. In embodiments, the elevated level of an inflammation marker is detected in the serum of a patient. In embodiments, the elevated level of an inflammation marker is detected in the lung of a patient. In embodiments, a patient has elevated levels of interleukin-6 (IL-6). In embodiments, a patient has elevated levels of interleukin-8 (IL-8). In embodiments, a patient has elevated levels of tumor necrosis factor α (TNF-α). In embodiments, a patient has elevated levels of C-reactive protein (CRP). In embodiments, a patient has elevated levels of gamma interferon (IFN-y)-inducible protein 10 (IP-10). In embodiments, a patient has a presence of ground-glass opacity. In embodiments, a HIF prolyl hydroxylase inhibitor or a HIF-alpha stabilizer is administered to a patient in need thereof, wherein the patient has a positive diagnostic test for SARS-CoV-2.

In embodiments, a patient has one or more of the following: reduced peripheral lymphocytes, elevated levels of C-reactive protein (CRP), elevated levels of IL-6, or abnormal coagulation parameters.

In embodiments, a patient has a pre-existing respiratory or pulmonary disease before the development of a disease or condition as described herein. Examples of pre-existing respiratory or pulmonary diseases include acute lung injury, bronchitis, pneumonia, pulmonary fibrosis, asthma, and acute respiratory distress syndrome, or pulmonary hypertension. In embodiments, a patient does not has a pre-existing respiratory or pulmonary disease before the development of a disease or condition as described herein.

In embodiments, a patient is a child. In embodiments, a patient is an adult. In embodiments, a patient is an adult aged 45 years and above. In embodiments, a patient is an adult aged 65 years and above. In embodiments, a patient is an adult aged 80 years and above. In embodiments, a patient is an adult aged 45-60 years. In embodiments, a patient is an adult aged 60-80 years. In embodiments, a patient is < about 65 years of age. In embodiments, a patient is ≥ about 65 years of age and < about 80 years of age. In embodiments, a patient is ≥ about 80 years of age.

In embodiments, a patient has serious underlying medical conditions. Examples of underlying medical conditions include liver disease, heart conditions, kidney disease, severe obesity, diabetes, and being immunocompromised. In embodiments, a patient has a body mass index (BMI) that is 25 or above or 30 or above.

In embodiment, a patient is / has 1, 2, 3, 4, 5, or all of the following: 1) men or women ≥18 years of age; 2) laboratory-confirmed diagnosis of COVID-19 by detection of SARS-CoV-2 RNA by RT-PCR from any specimen respiratory; 3) admitted to the hospital; 4) within 24 hours of hospital admission; 5) oxygen saturation of hemoglobin by pulse oximetry at room air ≤ 94%; and 6) has not participated in any other clinical trial for the treatment for COVID-19. In embodiment, a patient is not / has not any or all of the following: 1) hypersensitivity to vadadustat or any of its excipients; 2) placed on mechanical ventilation prior to commencement of treatment; 3) hemoglobin more than 1 g/dL above the gender specific upper limit of normal (ULN) prior to commencement of treatment; 4) aspartate aminotransferase (AST), alanine aminotransferase (ALT), or total bilirubin > 2.0 × ULN prior to commencement of treatment; 5) women who are pregnant or breastfeeding, or positive pregnancy test prior to commencement of treatment; 6) on maintenance dialysis prior to commencement of treatment; and 7) have received a solid organ transplant prior to commencement of treatment.

### Severity of the Disease

The primary endpoint should be responsive to the eligible patient population, intervention and course of illness of COVID-19. While all-cause mortality (ACM) is an important outcome, depending upon event rates observed in the Pilot Stage, the primary endpoint should be a composite measure of clinical improvement and/or survival, assessed at a pre-specified time (such as 28 days) post randomization. The National Institute of Allergy and Infectious Disease Ordinal Scale (NIAID-OS) can be used to measure illness severity over time. The primary outcome could be a measure of patients’ clinical status at a particular time point after enrolment, depending upon frequency of outcome assessment (e.g., 14, 28, or 60 days). Agreement and consistency in recording of individual outcome events at particular time points will facilitate interpretation and combination of results across studies and trials. The definition of the endpoint should be fine-tuned for the Pivotal Phase, based on the Pilot Phase of the trial.

In embodiments, the severity of a diseases is maintained or reduced following commencement of administering a HIF prolyl hydroxylase inhibitor or a HIF-alpha stabilizer to the patient as described herein.

Suitable HIF prolyl hydroxylase inhibitors or HIF-alpha stabilizers include (but are not limited to) those described herein, including compounds according to any one Formulas (I)-(VI), such as vadadustat (AKB-6548), roxadustat (FG-4592), daprodustat (GSK-12788363), molidustat (BAY 85-3934), enarodustat (JTZ-951), or desidustat (ZYAN1), or any pharmaceutically acceptable salt thereof. In embodiments, a compound is vadadustat.

In embodiments, a disease is COVID-19.

In embodiments, the severity of diseases such as COVID-19 may be assessed according to the following ordinal scale where increasing numbers denote increased severity. Methods described herein can be useful for infected patients having any of the characteristics described for any of scores 1-7, regardless of whether the patient is formally scored.

| Score | Descriptor | Patient State | |
|---|---|---|---|
| 0 | No clinical or virological evidence of infection | Uninfected | Uninfected/Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | Infected -Hospitalized (Severe Disease) | |
| 6 | Patient is hospitalized and receives oxygen therapy that is non-invasive ventilation or high flow oxygen devices | | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |
| 8 | Death | Dead | Death |

In embodiments, the severity of disease as described herein is maintained and the patient’s condition stabilizes following commencement of administering the HIF prolyl hydroxylase inhibitor or the HIF-alpha stabilizer to the patient. In embodiments, a patient’s score does not change and the patient’s condition stabilizes following commencement of administering the HIF prolyl hydroxylase inhibitor or the HIF-alpha stabilizer to the patient.

In embodiments, the severity of disease as described herein decreases following commencement of administering the HIF prolyl hydroxylase inhibitor or the HIF-alpha stabilizer to the patient. In embodiments, a patient’s score decreases by at least one point following commencement of administering the HIF prolyl hydroxylase inhibitor or the HIF-alpha stabilizer to the patient. In embodiments, a patient’s score decreases by one to three points following commencement of administering the HIF prolyl hydroxylase inhibitor or the HIF-alpha stabilizer to the patient. In embodiments, a patient’s score decreases by four to six points following commencement of administering the HIF prolyl hydroxylase inhibitor or the HIF-alpha stabilizer to the patient. In embodiments, a patient’s score decreases by six points or above following commencement of administering the HIF prolyl hydroxylase inhibitor or the HIF-alpha stabilizer to the patient.

In embodiments, a patient is assessed as uninfected/ambulatory. In embodiments, a patient is assessed as hospitalized but not intubated. In embodiments, a patient is assessed as hospitalized and intubated.

In embodiments, the severity of disease as described herein decreases following commencement of administering the HIF prolyl hydroxylase inhibitor or the HIF-alpha stabilizer to the patient. In embodiments, a patient’s category decreases by at least one category following commencement of administering the HIF prolyl hydroxylase inhibitor or the HIF-alpha stabilizer to the patient. In embodiments, a patient’s category decreases by two categories following commencement of administering the HIF prolyl hydroxylase inhibitor or the HIF-alpha stabilizer to the patient.

Exemplary decreased severity of a disease is described herein.

In embodiments, a patient is hypoxic. In embodiments, a patient exhibits one or more symptoms of hypoxia. In embodiments, the one or more symptoms include changes in the color of the skin, confusion, cough, fast heart rate, rapid breathing, shortness of breath, sweating, and/or wheezing. In embodiments, decreased severity of hypoxia includes the improvement in one or more symptoms described herein following commencement of administering the HIF prolyl hydroxylase inhibitor or the HIF-alpha stabilizer to the patient.

In embodiments, a patient has decreased oxygen saturation of hemoglobin relative to normal levels. In embodiments, a patient an oxygen saturation of hemoglobin that is 94% or less. In embodiments, decreased severity of a disease includes the improvement in oxygen saturation value. In embodiments, a patient’s oxygen saturation value improves to a value of about 95% following commencement of administering the HIF prolyl hydroxylase inhibitor or the HIF-alpha stabilizer to the patient. In embodiments, a patient’s oxygen saturation value improves to a value of about 95% or greater following commencement of administering the HIF prolyl hydroxylase inhibitor or the HIF-alpha stabilizer to the patient.

In embodiments, a patient has hypotension. In embodiments, a patient with hypotension receives vasopressor therapy. In embodiments, decreased severity of hypotension includes a reduced or discontinued vasopressor therapy following commencement of administering the HIF prolyl hydroxylase inhibitor or the HIF-alpha stabilizer to the patient.

In embodiments, a patient is hospitalized. In embodiments, a patient does not receive oxygen therapy. In embodiments, a patient has a score of 1, 2, and/or 3 according to the ordinal scale described herein. In embodiments, a decreased severity of the disease is a decrease of the patient’s score (e.g. decreases by at least one point) following commencement of administering the HIF prolyl hydroxylase inhibitor or the HIF-alpha stabilizer to the patient.

In embodiments, a patient is hospitalized. In embodiments, a patient receives oxygen therapy. In embodiments, a patient has a score of 4, 5, 6, or 7 according to the ordinal scale described herein. In embodiments, a decreased severity of the disease is a decrease of the patient’s score (e.g. decreases by at least one point, by one to three points, or by four points and above) following commencement of administering the HIF prolyl hydroxylase inhibitor or the HIF-alpha stabilizer to the patient.

In embodiments, a patient receives oxygen therapy. In embodiments, a patient receives oxygen therapy by mask or nasal prongs. In embodiments, a patient receives oxygen therapy by intubation and mechanical ventilation. In embodiments, a decreased severity of the disease is a discontinued oxygen therapy following commencement of administering the HIF prolyl hydroxylase inhibitor or the HIF-alpha stabilizer to the patient. In embodiments, a patient has increased ventilator-free survival following discontinuation of mechanical ventilation.

In embodiments, a patient has multi-organ injuries and/or diseases receives additional organ support therapy. In embodiments, a patient has any of a cardiovascular injury, a neurological injury, a liver injury, or a pancreas injury, and/or a kidney disease. In embodiments, a patient has a heart injury and/or a lung injury. In embodiments, a patient receives organ support therapy that is vapopressor therapy, renal replacement therapy (RRT), and/or extracorporeal membrane oxygenation (ECMO). In embodiments, a decreased severity of the disease is a discontinued organ support therapy following commencement of administering the HIF prolyl hydroxylase inhibitor or the HIF-alpha stabilizer to the patient. In embodiments, a patient has increased organ failure-free survival (e.g. kidney, cardiac) following commencement of administering the HIF prolyl hydroxylase inhibitor or the HIF-alpha stabilizer to the patient.

In embodiments, a patient receives vadadustat, or a pharmaceutically acceptable salt thereof.

### Effects/Efficacy of Treatment and Endpoints

Efficacy of any method described herein can be assessed according to methods known in the art. Exemplary endpoints and effects of treatment are described herein.

For example, efficacy of a method described herein may be assessed by a change of one point or more (from baseline) on an ordinal scale for clinical improvement (e.g., the NIAID-OS). A change may be assessed any time during the period during which a HIF prolyl hydroxylase inhibitor or the HIF-alpha stabilizer (e.g., vadadustat). In embodiments, a change is assessed on, at, or by day 28 of treatment. In embodiments, a change is assessed on, at, or by day 21 of treatment. In embodiments, a change is assessed on, at, or by day 14 of treatment. In embodiments, a change is assessed on, at, or by day 7 of treatment. A change may be an improvement by one point or more. A change may be a worsening by one point or more. For example, a change may be worsening by one point or more (from baseline) on the NIAID ordinal scale for clinical improvement (e.g., on or at day 7 or day 14).

In embodiments, efficacy is assessed by the proportion of patients with each of the components of the NIAID ordinal scale for clinical improvement at day 7 and 14.

In embodiments, efficacy is assessed by the proportion of patient population assessed according to the NIAID ordinal scale, including according to any of the following four categories: 1) uninfected/ambulatory, 2) hospitalized but not intubated, 3) hospitalized and intubated, or 4) death.

In embodiments, efficacy is assessed by the time to each component of the NIAID ordinal scale.

In embodiments, efficacy is assessed by the time to improvement of at least 2 categories relative to baseline on an ordinal scale (e.g., the NIAID ordinal scale of clinical improvement).

In embodiments, efficacy is assessed by the time to any of the components of an ordinal scale such as the NIAID ordinal scale. In embodiments, efficacy is assessed by the time to any of the following four categories: 1) uninfected/ambulatory, 2) hospitalized but not intubated, 3) hospitalized and intubated, or 4) death.

In embodiments, efficacy is assessed by the proportion of patients with clinical diagnosis of ARDS.

In embodiments, efficacy is assessed by time to clinical diagnosis of ARDS.

In embodiments, efficacy is assessed by ventilator-free survival.

In embodiments, efficacy is assessed by organ failure-free survival (e.g., kidney, cardiac).

In embodiments, efficacy is assessed by a change in serum creatinine from baseline.

In embodiments, efficacy is assessed by overall total survival.

In embodiments, efficacy is assessed by the proportion of patients with clinical diagnosis of sepsis.

In embodiments, efficacy is assessed by the time to clinical failure. In embodiments, clinical failure is mechanical ventilation. In embodiments, clinical failure is mechanical ventilation. In embodiments, clinical failure is death or mechanical ventilation, whichever occurs first.

In embodiments, efficacy is assessed by the time to hospital discharge.

In embodiments, efficacy is assessed by the proportion of patients with acute kidney injury.

In embodiments, efficacy is assessed by the time to acute kidney injury.

In embodiments, efficacy is assessed by inducing known transcriptional targets of HIFs (e.g., erythropoietin, extracellular adenosine levels, circulating levels of CD73 (or ecto-5′-nucleotidase) VEGF (vascular endothelial growth factor), hypoxia-driven microRNAs that dampen lung inflammation).

In embodiments, efficacy is assessed using various laboratory measurements that include but not limited to serum creatinine, FGF-23 (or fibroblast growth factor 23), and markers of attenuated inflammation.

In embodiments, a method described herein (e.g., for the prevention and treatment of ARDS among COVID-19 patients) results in a change (e.g., a reduction by ≥1 point) on the NIAID ordinal scale for clinical improvement at day seven or day 14 of treatment.

NIAID Ordinal Scale (NIAID-OS)

| Score | Descriptor | Patient State | |
|---|---|---|---|
| 0 | No clinical or virological evidence of infection | Uninfected | Uninfected/Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | Infected -Hospitalized (Severe Disease) | |
| 6 | Patient is hospitalized and receives oxygen therapy that is non-invasive ventilation or high flow oxygen devices | | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |
| 8 | Death | Dead | Death |

In embodiments, a method described herein (e.g., for the prevention and treatment of ARDS among COVID-19 patients such as hospitalized COVID-19 patients) results in a change (e.g., a decrease or a reduction) in the proportion of patients at day 14 who are dead (NIAID-OS 8); hospitalized, on invasive mechanical ventilation or ECMO (NIAID-OS 7); and/or hospitalized, on non-invasive ventilation or high flow oxygen devices (NIAID-OS 6).

In embodiments, a method described herein (e.g., for the prevention and treatment of ARDS among COVID-19 patients such as hospitalized COVID-19 patients) results in a change (e.g., an increase) in the proportion of patients with a score of 0 at 14 days on the MSOFA (Modified Sequential Organ Failure Assessment) scale:

| | | | | | |
|---|---|---|---|---|---|
| Organ System | 0 | 1 | 2 | 3 | 4 |
| Respiratory SpO2/FiO2 | > 400 | ≤ 400 | ≤ 315 | ≤ 235 | ≤ 150 |
| Liver | No scleral icterus or jaundice | | | Scleral icterus or jaundice | |
| Cardiovascular , hypotension | No hypotensio n | MAP < 70 m m Hg | Dopamine ≤ 5 or dobutamin e any dose | Dopamine > 5 Epinephrine ≤ 0.1 Norepinephrin e ≤ 0.1 | Dopamine > 15 Epinephrine > 0.1 Norepinephrin e > 0.1 |
| CNS (central nervous system), Glasgow Coma Score | 15 | 13-14 | 10-12 | 6-9 | < 6 |
| Renal, Creatinine mg/dL | < 1.2 | 1.2 - 1.9 | 2.0 - 3.4 | 3.5 - 4.9 | > 5.0 |

Note: Discharged patient receives a score of 0 and dead patient receives a score of 20.

In embodiments, a method described herein (e.g., for the prevention and treatment of ARDS among COVID-19 patients such as hospitalized COVID-19 patients) results in a change (e.g., a decrease or a reduction) in the average MSOFA scores at day 7 and/or day 14.

In embodiments, a method described herein (e.g., for the prevention and treatment of ARDS among COVID-19 patients such as hospitalized COVID-19 patients) results in a change (e.g., a decrease or a reduction) in the proportion of patients who develop ARDS or experienced increased ARDS severity from baseline based on the Berlin scale.

In embodiments, a method described herein (e.g., for the prevention and treatment of ARDS among COVID-19 patients such as hospitalized COVID-19 patients) results in a change (e.g., an increase) in the ventilator-free survival at day 7 and day 14.

In embodiments, a method described herein (e.g., for the prevention and treatment of ARDS among COVID-19 patients such as hospitalized COVID-19 patients) results in a change (e.g., an increase) in the overall survival at day 7 and/or day 14.

In embodiments, a method described herein (e.g., for the prevention and treatment of ARDS among COVID-19 patients such as hospitalized COVID-19 patients) results in a change (e.g., a decrease or a reduction) in the proportion of patients with hypotension (MAP (Mean Arterial Pressure) <70 mm Hg or requirement for inotropes or vasopressors to maintain blood pressure) at day 7 and/or day 14.

In embodiments, a method described herein (e.g., for the prevention and treatment of ARDS among COVID-19 patients such as hospitalized COVID-19 patients) results in a change (e.g., a decrease or a reduction) in the proportion of patients with acute kidney injury at day 7 and day 14.

In embodiments, a method described herein (e.g., for the prevention and treatment of ARDS among COVID-19 patients such as hospitalized COVID-19 patients) results in a change (e.g., a decrease or a reduction) in the time to hospital discharge.

Efficacy of vadadustat may also be assessed using still other measures, including effects on: inducing known transcriptional targets of HIFs (e.g., erythropoietin, extracellular adenosine levels, circulating levels of CD73 (or ecto-5′-nucleotidase), VEGF (vascular endothelial growth factor), hypoxia-driven microRNAs that dampen lung inflammation). Various laboratory measurements include but not limited to serum creatinine, FGF-23 (or fibroblast growth factor 23), and markers of attenuated inflammation.

### Pharmaceutical Compositions

Pharmaceutical compositions may be used in the preparation of individual, single unit dosage forms. Pharmaceutical compositions and dosage forms provided herein comprise a HIF prolyl hydroxylase inhibitor or a HIF-alpha stabilizer, such as a compound described herein ( e.g., a compound having a structure according to any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof). Pharmaceutical compositions and dosage forms can further comprise one or more excipients. Like the amounts and types of excipients, the amounts and specific types of active ingredients in a dosage form may differ depending on factors including, but not limited to, the route by which it is to be administered to patients.

In embodiments, a HIF prolyl hydroxylase inhibitor or a HIF-alpha stabilizer may be administered by oral or parenteral route. As used herein, the term “parenteral” includes but not limited to subcutaneous, intradermal, intravascular injections, such as intravenous, intramuscular and any another similar injection or infusion technique; transdermal route; inhalation, and rectal route. In embodiments, a HIF prolyl hydroxylase inhibitor or a HIF-alpha stabilizer may be administered orally, such as in a tablet or capsule formulation. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

### Oral Formulations

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In one embodiment, oral dosage forms are tablets or capsules, in which case solid excipients are employed. In another embodiment, tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM. Other suitable forms of microcrystalline cellulose include, but are not limited to, silicified microcrystalline cellulose, such as the materials sold as PROSOLV 50, PROSOLV 90, PROSOLV HD90, PROSOLV 90 LM, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in one embodiment, present in from about 50 to about 99 weight percent of the pharmaceutical composition or dosage form.

In embodiments, fillers may include, but are not limited to block copolymers of ethylene oxide and propylene oxide. Such block copolymers may be sold as POLOXAMER or PLURONIC, and include, but are not limited to POLOXAMER 188 NF, POLOXAMER 237 NF, POLOXAMER 338 NF, POLOXAMER 437 NF, and mixtures thereof.

In embodiments, fillers may include, but are not limited to isomalt, lactose, lactitol, mannitol, sorbitol xylitol, erythritol, and mixtures thereof.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In one embodiment, pharmaceutical compositions comprise from about 0.5 weight percent to about 15 weight percent of disintegrant, or from about 1 to about 5 weight percent of disintegrant.

Disintegrants that can be used in pharmaceutical compositions and dosage forms include, but are not limited to, agar-agar, alginic acid, calcium carbonate, microcrystalline cellulose, croscarmellose sodium, povidone, crospovidone, polacrilin potassium, sodium starch glycolate, potato or tapioca starch, other starches, pre-gelatinized starch, other starches, clays, other algins, other celluloses, gums, and mixtures thereof.

Glidants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium stearyl fumarate, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional glidants include, for example, a syloid silica gel (AEROSIL200, manufactured by W.R. Grace Co. of Baltimore, MD), a coagulated aerosol of synthetic silica (marketed by Degussa Co. of Plano, TX), CAB-O-SIL (a pyrogenic colloidal silicon dioxide product sold by Cabot Co. of Boston, MA), and mixtures thereof. If used at all, glidants may be used in an amount of less than about 1 weight percent of the pharmaceutical compositions or dosage forms into which they are incorporated.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

### Doses and Dosing Regimens

In embodiments, a disease or condition as described herein, may be treated by administering to a patient in need thereof from about 100 mg to about 1800 mg, about 100 mg to about 1500 mg, about 150 mg to about 1200 mg, about 150 mg to about 1450 mg, about 200 mg to about 1400 mg, about 250 mg to about 1350 mg, about 300 mg to about 1300 mg, about 350 mg to about 1250 mg, about 400 mg to about 1200 mg, about 450 mg to about 1150 mg, about 500 mg to about 1100 mg, about 550 mg to about 1050 mg, about 600 mg to about 1000 mg, about 650 mg to about 950 mg, about 700 mg to about 900 mg, about 850 mg to about 950 mg, about 800 mg to about 1000 mg, about 750 mg to about 1050 mg, about 700 mg to about 1100 mg, about 650 mg to about 1150 mg, about 600 mg to about 1200 mg, about 150 mg to about 1800 mg, about 300 mg to about 1800 mg, about 450 mg to about 1800 mg, about 600 mg to about 1800 mg, about 750 mg to about 1800 mg, or about 900 mg to about 1800 mg of a HIF prolyl hydroxylase inhibitor or a HIF-alpha stabilizer. In embodiments, the dose of a HIF prolyl hydroxylase inhibitor or a HIF-alpha stabilizer is between 150 mg to 750 mg. In embodiments, the dose of a HIF prolyl hydroxylase inhibitor or a HIF-alpha stabilizer is between 900 mg to 1800 mg. In embodiments, the dose of a HIF prolyl hydroxylase inhibitor or a HIF-alpha stabilizer is between 150 mg to 1800 mg. In embodiments, the dose of a HIF prolyl hydroxylase inhibitor or a HIF-alpha stabilizer is between 150 mg to 1000 mg. In embodiments, the dose of a HIF prolyl hydroxylase inhibitor or a HIF-alpha stabilizer is about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1000 mg, about 1020 mg, about 1040 mg, about 1060 mg, about 1080 mg, about 1100 mg, about 1120 mg, about 1140 mg, about 1160 mg, about 1180 mg, about 1200 mg, about 1220 mg, about 1240 mg, about 1260 mg, about 1280 mg, about 1300 mg, about 1320 mg, about 1340 mg, about 1360 mg, about 1380 mg, about 1400 mg, about 1420 mg, about 1440 mg, about 1460 mg, about 1480 mg, about 1500 mg, about 1650 mg, or about 1800 mg. In embodiments, such doses may be administered orally, three times a week, once daily, twice daily, or three times daily. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the dose of a HIF prolyl hydroxylase inhibitor or a HIF-alpha stabilizer is about 150 mg to about 1500 mg, about 150 mg to about 1200 mg, about 150 mg to about 1800 mg, or about 900 mg to about 1800 mg. In embodiments, the dose of a HIF prolyl hydroxylase inhibitor or a HIF-alpha stabilizer is about 150 mg, about 300 mg, about 450 mg, about 600 mg, about 750 mg, about 900 mg, about 1050 mg, about 1200 mg, about 1350 mg, about 1500 mg, about 1650 mg, or about 1800 mg. In embodiments, the initial dose of a HIF prolyl hydroxylase inhibitor or a HIF-alpha stabilizer is about 900 mg. In embodiments, such doses may be administered orally, three times a week, once daily, twice daily, or three times daily. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, the dose of vadadustat is between about 100 mg to about 1800 mg, about 100 mg to about 1500 mg, about 150 mg to about 1450 mg, about 200 mg to about 1400 mg, about 250 mg to about 1350 mg, about 300 mg to about 1300 mg, about 350 mg to about 1250 mg, about 400 mg to about 1200 mg, about 450 mg to about 1150 mg, about 500 mg to about 1100 mg, about 550 mg to about 1050 mg, about 600 mg to about 1000 mg, about 650 mg to about 950 mg, about 700 mg to about 900 mg, about 850 mg to about 950 mg, about 800 mg to about 1000 mg, about 750 mg to about 1050 mg, about 700 mg to about 1100 mg, about 650 mg to about 1150 mg, about 600 mg to about 1200 mg, about 150 mg to about 1800 mg, about 300 mg to about 1800 mg, about 450 mg to about 1800 mg, about 600 mg to about 1800 mg, about 750 mg to about 1800 mg, or about 900 mg to about 1800 mg. In embodiments, the dose of vadadustat is between 150 mg to 750 mg. In embodiments, the dose of vadadustat is between 150 mg to 1000 mg. In embodiments, the dose of vadadustat is between 150 mg to 1800 mg. In embodiments, the dose of vadadustat is between 900 mg to 1800 mg. In embodiments, the dose of vadadustat is about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, about 1000 mg, about 1020 mg, about 1040 mg, about 1060 mg, about 1080 mg, about 1100 mg, about 1120 mg, about 1140 mg, about 1160 mg, about 1180 mg, about 1200 mg, about 1220 mg, about 1240 mg, about 1260 mg, about 1280 mg, about 1300 mg, about 1320 mg, about 1340 mg, about 1360 mg, about 1380 mg, about 1400 mg, about 1420 mg, about 1440 mg, about 1460 mg, about 1480 mg, about 1500 mg, about 1650 mg, or about 1800 mg. In embodiments, such doses may be administered orally, three times a week, once daily, twice daily, or three times daily.

In embodiments, the dose of vadadustat is about 150 mg, about 300 mg, about 450 mg, about 600 mg, about 750 mg, about 900 mg, about 1050 mg, about 1200 mg, about 1350 mg, about 1500 mg, about 1650 mg, or about 1800 mg. In embodiments, the initial dose of vadadustat is about 900 mg. In embodiments, such doses may be administered orally, three times a week, once daily, twice daily, or three times daily.

In embodiments, a dose of vadadustat is about 900 mg. For example, a method for treating or preventing acute respiratory distress syndrome (ARDS) can comprise administering to a patient in need thereof (e.g., a patient with COVID-19) about 900 mg vadadustat. In embodiments, a patient is hospitalized. In embodiments, an initial dose of vadadustat administered to a patient is about 900 mg. In embodiments, a dose is administered daily. In embodiments, a dose is administered once daily. In embodiments, a dose of vadadustat is adjusted by about 150 mg (e.g., lowered by about 150 mg or increased by about 150 mg).

In embodiments, a dose of vadadustat is about 900-1800 mg. For example, a method for treating or preventing acute respiratory distress syndrome (ARDS) can comprise administering to a patient in need thereof (e.g., a patient with COVID-19) about 900-1800 mg vadadustat. In embodiments, a patient is hospitalized. In embodiments, a dose is administered daily. In embodiments, a dose is administered once daily. In embodiments, a dose of vadadustat is adjusted by about 150 mg (e.g., lowered by about 150 mg or increased by about 150 mg), optionally where the maximum dose is about 1800 mg. In embodiments, a dose (e.g., a daily dose) of vadadustat is about 900, 1050, 1200, 1350, 1500, 1650, or 1800 mg.

In embodiments, a HIF prolyl hydroxylase inhibitor or HIF-alpha stabilizer is administered to a patient for up to about one month. In embodiments, a HIF prolyl hydroxylase inhibitor or the HIF-alpha stabilizer is administered to a patient for up to about four weeks (about 28 days), three weeks (about 21 days), two weeks (about 14 days), or one week (about 7 days). In embodiments, a HIF prolyl hydroxylase inhibitor or the HIF-alpha stabilizer is administered to a patient for up to about two weeks (about 14 days). Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

In embodiments, a HIF prolyl hydroxylase inhibitor or HIF-alpha stabilizer is administered orally. In embodiments, a HIF prolyl hydroxylase inhibitor or HIF-alpha stabilizer is administered to an intubated patient through a nasogastric tube. Examples of HIF prolyl hydroxylase inhibitors or HIF-alpha stabilizers include compounds of any one of Formulas (I)-(VI), or a compound such as vadadustat (AKB-6548), roxadustat (FG-4592), daprodustat (GSK-12788363), molidustat (BAY 85-3934), enarodustat (JTZ-951), or desidustat (ZYAN1), or a pharmaceutically acceptable salt thereof. In embodiments, a compound is vadadustat.

As an exemplary embodiment, a patient can receive a prolyl hydroxylase inhibitor or HIF-alpha stabilizer (e.g., oral administration of vadadustat) once daily (e.g., for 14 days). In embodiments, a patient (e.g., a patient having COVID-19) can receive said daily dose in order to treat or prevent Acute Respiratory Distress Syndrome (ARDS). In embodiments, a patient is hospitalized. In embodiments, a patient can receive a dose of about 150 mg-1800 mg vadadustat (e.g., about 900-1800 mg vadadustat). In embodiments, a patient receives an initial daily dose is about 900 mg vadadustat and/or receives a dose of about 900 mg vadadustat throughout the period of treatment. Vadadustat can be administered in unit dosage forms (e.g., a tablet) comprising about 75, 150, 300, 450, or 600 mg of the drug.

### Examples

### Example 1. Methods of Treating Patients Having COVID-19

Methods described herein can be useful for treating patients infected by the novel severe acute respiratory syndrome coronavirus 2 (SARS-CoV-2) and having COVID-19. The present example illustrates a method of treating or preventing COVID-19 related pneumonia and related conditions including acute lung injury (ALI), acute respiratory distress syndrome (ARDS), and associated organ failure (e.g., as described herein).

Patients who can receive therapy comprising administration of a compound or pharmaceutically acceptable salt thereof that is a HIF prolyl hydroxylase inhibitor or a HIF-alpha stabilizer (e.g., vadadustat) include adult patients with evidence of hypoxia such as an oxygen saturation level that is 94% or less. An adult patient can be about 45 years of age or about (e.g., about 45-60 years of age, about 60-80 years of age, or over about 80 years of age).

Patients can also be identified by the severity of their disease, including as described by the below ordinal scale. For example, a patient can be assessed according to the following ordinal scale or exhibit the severity of disease as described by any of scores 2-7 of this scale.

| Score | Descriptor | Patient State | |
|---|---|---|---|
| 0 | No clinical or virological evidence of infection | Uninfected | Uninfected/Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | Infected -Hospitalized (Severe Disease) | |
| 6 | Patient is hospitalized and receives oxygen therapy that is non-invasive ventilation or high flow oxygen devices | | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |
| 8 | Death | Dead | Death |

Patients can receive daily doses of the compound (e.g., vadadustat) of about 100-1500 mg/day. The patient can receive therapy for time periods as described herein, including up to about one month (e.g., up to about 4 weeks (about 28 days), about 3 weeks (about 21 days), about 2 weeks (about 14 days), or about 1 week (about 7 days)). In embodiments, a patient receives a dose (e.g., a daily dose) of the compound (e.g., vadadustat) for up to about 2 weeks (about 14 days). In embodiments, a patient receives a dose of about 900 mg of vadadustat. In embodiments, a patient receives an initial dose of about 900 mg of vadadustat. In embodiments, a patient receives a dose of up to about 1800 mg of vadadustat (e.g., about 150, 300, 450, 600, 750, 900, 1050, 1200, 1350, 1500, 1650, or 1800 mg vadadustat or about 900 to about 1800 mg vadadustat).

Following commencement of treatment, the severity of disease may be maintained such that the patient’s condition can stabilize. For example, a patient’s score according to the ordinal scale can remain unchanged such that the patient’s condition stabilizes following treatment with the HIF prolyl hydroxylase inhibitor or the HIF-alpha stabilizer (e.g., vadadustat) to the patient.

The severity of disease may decrease following commencement of administering the HIF prolyl hydroxylase inhibitor or the HIF-alpha stabilizer to the patient. For example, a patient’s score as assessed by the ordinal scale can decrease by at least one point following commencement of administering the HIF prolyl hydroxylase inhibitor or the HIF-alpha stabilizer (e.g., vadadustat).

Further, the methods provided herein can treat or prevent other organ injury (including multi-organ injury) in a patient. Additional benefits of the treatment can include any of the following, or any combination thereof, following commencement of treatment:
a patient’s oxygen saturation value can improve to a value of about 95% or greater;
an oxygen therapy received by the patient can be discontinued (e.g., mechanical ventilation is discontinued and the patient experiences increased ventilator-free survival or a patient no longer requires an oxygen therapy comprising a face mask, high flow oxygen, noninvasive mechanical ventilation, or intubation);
a vasopressor therapy is reduced or discontinued; and/or
a patient whose hypotension exhibits clinical improvement.

Still further benefits of the treatment can include any of those described herein. For example, described herein is an exemplary method for treating or preventing acute respiratory distress syndrome (ARDS) in a patient having COVID-19.

### Example 1a. Methods of Treating Patients Having COVID-19

This example describes use of 5-(3-chlorophenyl)-3-hydroxypyridine-2-carbonyl] amino} acetic acid (vadadustat) in a multicenter, randomized, double-blind, placebo-controlled Bayesian adaptive clinical for the prevention and treatment of acute respiratory distress syndrome (ARDS) in hospitalized patients with coronavirus disease 2019 (COVID-19).

Patients may be divided into the following age categories:
<65 years,
≥ 65 and < 80 years, and
≥80 years.

### Selection and Withdrawal of Subjects

Subjects may be selected for treatment based on the certain inclusion and exclusion criteria, including any or all of the exemplary criteria described herein.

Inclusion criteria may include any or all of the following:
1. Men and women ≥ 18 years of age.
2. Laboratory-confirmed diagnosis of COVID-19 by detection of SARS-CoV-2 RNA by RT-PCR (reverse transcription polymerase chain reaction) from any specimen respiratory.
3. Admitted to the hospital.
4. Within 24 hours of hospital admission.
5. Oxygen saturation of hemoglobin by pulse oximetry at room air <94%.
6. Participation in any other clinical trial for the treatment for COVID-19 is not allowed.
7. Understands the procedures and requirements of treatment and provides written informed consent and authorization for protected health information disclosure.

Exclusion may include any or all of the following:
1. Males with hemoglobin >17.5 g/dL or females >15.5 g/dL.
2. Hypersensitivity to vadadustat or any of its excipients.
3. Placed on mechanical ventilation before randomization.
4. Hemoglobin above the gender specific upper limit of normal (ULN) at randomization.
5. Aspartate aminotransferase (AST), alanine aminotransferase (ALT), or total bilirubin >2.0 × ULN at randomization.
6. Patients who have erythrocytosis or polycethemia vera.
7. Patients receiving any of the following medications: sulfasalazine or other BCRP substrates, probenecid or other UGT and OAT1/3 inhibitors.
8. Women who are pregnant or breastfeeding, or positive pregnancy test before randomization.
9. Patients on maintenance dialysis.
10. Patients who have received a solid organ transplant.
11. Patients who are prisoners.

Vadadustat can be administered to a patient according to methods described herein. In some embodiments, vadadustat may be permanently discontinued if a patient meets any of the following prespecified criteria:
Patient completes 14 days of in-hospital treatment.
Patient is discharged before 14 days of treatment.
Patient dies.
If the patients develops ALT or AST >3x ULN or total bilirubin >3x ULN.
If the patient hemoglobin level are > 17.5 g/dl for males or >15.5 g/dl for females.
Other reasons:
Unacceptable toxicity or drug intolerability.
Patient withdrawal of consent.
Patient becomes pregnant.

### Study Design

Following screening, patients are randomized in a 1:1 ratio to either vadadustat 900 mg or placebo. Randomination is stratified by site, and MSOFA score at baseline (<4 and ≥4).

### Treatment of Subjects

A subject may receive vadadustat (e.g., 900 mg vadadustat as 6 tablets once daily) for 14 days and without regard for meals at approximately the same time each day. Tablets can be crushed using a pill crusher and administered to an intubated patient through a nasogastric tube.

If a patient is receiving additional therapeutic agents concurrently with vadadustat therapy, doses of the additional therapeutic agents may be adjusted. For example, doses of the following concomitant medication may be adjusted during treatment as follows:
1. Simvastatin, maximum daily dose of 20 mg.
2. Rosuvastatin, maximum daily dose of 10 mg.
3. If the patient is currently taking erythropoiesis-stimulating agents or ESA (e.g., epoetin alfa, darbepoietin, or methoxy polyethylene glycol-epoetin beta), the medication should be discontinued.

All hospitalized patients with COVID-19 should receive prophylactic anticoagulation with LMWH (Low Molecular Weight Heparin) if no contraindications:
1. If CrCl (Creatinine clearance) >30 mL/min - use lovenox 40 mg sc qd.
2. If CrCl <30 mL/min - use UFH (unfractionated heparin).
3. In obese patients, increase lovenox dose to 40 mg bid.
4. If history of HIT (heparin-induced thrombocytopenia) or HITT (heparin-induced thrombocytopenia / thrombosis), use non heparin alternative.
5. If contraindications for AC (anticoagulation), do SCDs (sequential compression devices).

Patients can be identified by the severity of their disease, including as described herein, including according to the below scale based on the National Institute of Allergy and Infectious Disease Ordinal Scale (NIAID-OS).

| Score | Descriptor | Patient State | |
|---|---|---|---|
| 0 | No clinical or virological evidence of infection | Uninfected | Uninfected/Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | Infected -Hospitalized (Severe Disease) | |
| 6 | Patient is hospitalized and receives oxygen therapy that is non-invasive ventilation or high flow oxygen devices | | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |
| 8 | Death | Dead | Death |

For example, the efficacy of vadadustat (e.g., 900 mg per day) for the prevention and treatment of ARDS among COVID-19 patients may be measured by ≥ 1 point deterioration on the NIAID ordinal scale for clinical improvement at day seven or day 14 of treatment.

Efficacy of treatment can be assessed as described herein.

For example, efficacy may be assessed by a change of one point or more (from baseline) on the National Institute of Allergy and Infectious Disease Ordinal Scale (NIAID-OS) for clinical improvement. A change may be assessed on, at, or by day 7 of treatment or on, at, or by day 14 of treatment. A change may be an improvement by one point or more. A change may be a worsening by one point or more. For example, a change may be worsening by one point or more (from baseline) on the NIAID ordinal scale for clinical improvement (e.g., on or at day 7 or day 14).

Efficacy may also be assessed according to any of the following (including any combination thereof):
1. The proportion of patients at day 14 who are dead (8) or hospitalized, on invasive mechanical ventilation or ECMO (7) or hospitalized, on non-invasive ventilation or high flow oxygen devices (6) on the NIAID-OS.
2. Proportions of patients with a score of 0 at 14 days on the MSOFA (Modified Sequential Organ Failure Assessment) scale:

| | | | | | |
|---|---|---|---|---|---|
| Organ System | 0 | 1 | 2 | 3 | 4 |
| Respiratory SpO2/FiO2 | > 400 | ≤ 400 | ≤ 315 | ≤ 235 | ≤ 150 |
| Liver | No scleral icterus or jaundice | | | Scleral icterus or jaundice | |
| Cardiovascular, hypotension | No hypotension | MAP < 70 mm Hg | Dopamine ≤ 5 or dobutamine any dose | Dopamine > 5 Epinephrine ≤ 0.1 Norepinephrine ≤ 0.1 | Dopamine > 15 Epinephrine > 0.1 Norepinephrine > 0.1 |
| CNS (central nervous system), Glasgow Coma Score | 15 | 13 - 14 | 10 - 12 | 6-9 | <6 |
| Renal, Creatinine mg/dL | < 1.2 | 1.2 - 1.9 | 2.0 - 3.4 | 3.5 - 4.9 | > 5.0 |

Note: Discharged patient receives a score of 0 and dead patient receives a score of 20.

3. Average MSOFA scores at day 7 and day 14.
4. Proportion of patients who develop ARDS or experienced increased ARDS severity from baseline based on the Berlin scale.
5. Ventilator-free survival at day 7 and day 14.
6. Overall survival at day 7 and day 14.
7. Proportion of patients with hypotension (MAP (Mean Arterial Pressure) <70 mm Hg or requirement for inotropes or vasopressors to maintain blood pressure) at day 7 and day 14.
8. Proportion of patients with acute kidney injury at day 7 and day 14.
9. Time to hospital discharge.

Results from the trial can indicate at least one of the following: a lower or a reduction in the proportion of patients at day 14 who are dead (8) or hospitalized, on invasive mechanical ventilation or ECMO (7) or hospitalized, on non-invasive ventilation or high flow oxygen devices (6) on the NIAID-OS for patients on vadadustat compared to placebo; an increase in the proportions of patients with a score of 0 at 14 days on the MSOFA for patients on vadadustat compared to placebo; a lower or a reduction in the average MSOFA scores at day 7 and day 14 for patients on vadadustat compared to placebo; a lower or a reduction in the proportion of patients who develop ARDS or experienced increased ARDS severity from baseline based on the Berlin scale for patients on vadadustat compared to placebo; a greater or an increase in the number of ventilator-free survival at day 7 and day 14 for patients on vadadustat compared to placebo; a greater or an increase in the number of overall survival at day 7 and day 14 for patients on vadadustat compared to placebo; a lower or a reduction in the proportion of patients with hypotension (MAP (Mean Arterial Pressure) <70 mm Hg or requirement for inotropes or vasopressors to maintain blood pressure) at day 7 and day 14 for patients on vadadustat compared to placebo; a lower or a reduction in the proportion of patients with acute kidney injury at day 7 and day 14 for patients on vadadustat versus placebo; and a shorter or a reduction in the time to hospital discharge for patients on vadadustat versus placebo.

Efficacy of vadadustat may also be assessed using still other measures, including effects on: inducing known transcriptional targets of HIFs (e.g., erythropoietin, extracellular adenosine levels, circulating levels of CD73 (or ecto-5′-nucleotidase), VEGF (vascular endothelial growth factor), hypoxia-driven microRNAs that dampen lung inflammation). Various laboratory measurements include but not limited to serum creatinine, FGF-23 (or fibroblast growth factor 23), and markers of attenuated inflammation.

### Example 2. Effect of Compound 1 (Vadadustat) in LPS-Induced Lung Inflammation Model in Mice

The purpose of this study is to evaluate the effects of Compound 1 in LPS-induced lung model of inflammation in mice.

Acute lung injury (ALI) and its more severe condition, acute respiratory distress syndrome (ARDS), is an inflammatory lung disease characterized by inflammatory infiltrates, alveolar damage, hypoxemia, protein-rich edema, decreased lung compliance, and hyaline membrane formation (Altemeier, 2017; De Souza Xavier Costa, 2017; Hu, 2017; Li, 2016; Matute-Bello, 2008; Ye, 2005).

Currently, there are little to no pharmacological approaches available for the treatment of ALI/ARDS, and the fundamental mechanisms that initiate and propagate lung injury are still not fully understood (Li, 2016; Mabley, 2011; Matute-Bello, 2008; Patel, 2012; Ye, 2005). Therefore, this study has been designed to evaluate Compound 1 as a novel therapy for ALI/ARDS.

### Materials and Methods

A study was performed to evaluate the effects of test article Compound 1 (vadadustat) in a model of lipopolysaccharide (LPS)-induced pulmonary inflammation in male Swiss Webster mice. On study day -2, the animals were randomized into groups based on body weight. On study day 0, the mice were briefly anesthetized with 3% Isoflurane (same duration for all animals), and 50 µL of phosphate buffered saline (PBS, group 1) or 1 mg/mL LPS (groups 2-11) was instilled into the nose dropwise via a pipette. The animals were kept in an upright position for 2 minutes to allow for the fluid to trickle into the lungs; afterwards, the animals were returned to their home cage for recovery. The animals were treated by the oral (PO) route with vehicle (0.25% hydroxypropyl methylcellulose [HPMC], 0.1% Tween80) or Compound 1 (100 or 300 mg/kg) administered daily (QD) on days -2 through 0 (dosed 1 h pre-LPS or dosed 1 h pre- and 7 h post-LPS [8 h post-previous dose] on day 0) or administered once (1x) on day 0 (1 h post-LPS). Positive control mice were dosed PO, 1x on day 0 (1 h pre-LPS) with dexamethasone (3 mg/kg). Animal body weights were collected on study days -2 and 0. The mice were euthanized for necropsy and sample collection on study day 0 at 6 hours post-LPS (groups 1-4 and 8) or 8 hours post-LPS (groups 9-11) or on study day 1 at 24 hours post-LPS (groups 5-7). At necropsy, bronchoalveolar lavage (BAL) fluids and lung samples were collected for analysis, and plasma samples were collected for shipment to the study sponsor. Efficacy was evaluated based on absolute nucleated cell counts and cell differentials (lymphocytes, monocytes, neutrophils, eosinophils, and basophils), concentrations of the cytokines IL-6 and TNFαin BAL fluids, and histopathologic evaluation of the lungs. All animals survived to study termination.

Compound 1 was prepared for PO dosing at 10 mL/kg in vehicle: 0.25% HPMC (Sigma, catalogue No. H7509, lot No. HKCK737359), 0.1% Tween80 (Sigma, catalogue No. P1754, lot No. BCCF1555). Inotiv Boulder supplied the dexamethasone (VetOne, lot No. 200745), which was prepared in 1% carboxymethylcellulose (CMC: Inotiv Boulder, batch No. 2021, lot No. 2) for PO dosing at 10 mL/kg. The test articles were stored at room temperature protected from light when not in use.

Inotiv Boulder supplied the LPS serotype O111:B4 (Sigma, catalogue No. L2630-10MG, lot No. 099M4002V), which was prepared by mixing 1:1 in phosphate buffered saline (w/o Ca and Mg: Gibco, catalogue No. 10010-023, lot No. 2235048) to form a 1 mg/mL LPS solution.

Male Swiss Webster mice (N = 88 mice + 10 extra) that were 7 to 9 weeks of age were obtained from Charles River Laboratories (Wilmington, MA). The mice weighed approximately 29 to 45 grams (mean of 37 g) on study day -2.

### Experimental Design

On study day -2, the animals were randomized into groups based on body weight. On study day 0, the mice were briefly anesthetized with 3% Isoflurane (same duration for all animals), and 50 µL of PBS (group 1) or 1 mg/mL LPS (groups 2-11) was instilled into the nose dropwise via a pipette. The animals were kept in an upright position for 2 minutes to allow for the fluid to trickle into the lungs; afterwards, the animals were returned to their home cage for recovery. Dosing was initiated on -2 (groups 1-7) or day 0 (groups 8-11) and continued as indicated in Table 1. Body weight measurements were collected and the animals were euthanized for necropsy and terminal sample collection on day 0 or day 1.

TABLE 1

Study Group Designations:

| Grp | N | Disease | Treatment | Dose Level (mg/kg) | Dose Vol. (ml/kg)³ | Dose Conc. (mg/ml) | Dose Route | Regimen¹ , ² |
|---|---|---|---|---|---|---|---|---|
| 1 | 8 | PBS | Vehicle | -- | 10 | | PO | QD (Start day -2)* |
| 2 | 8 | LPS | Vehicle | -- | 10 | -- | PO | QD (Start day -2)* |
| 3 | 8 | LPS | Compound 1 | 100 | 10 | 10 | PO | QD (Start day -2)* |
| 4 | 8 | LPS | Compound 1 | 300 | 10 | 30 | PO | QD (Start day -2)* |
| 5 | 8 | LPS | Vehicle | -- | 10 | -- | PO | QD (Start day -2)** |
| 6 | 8 | LPS | Compound 1 | 100 | 10 | 10 | PO | QD (Start day -2)** |
| 7 | 8 | LPS | Compound 1 | 300 | 10 | 30 | PO | QD (Start day -2)** |
| 8 | 8 | LPS | Dexamethasone | 3 | 10 | 0.3 | PO | 1x (1 hr prior to LPS) |
| 9 | 8 | LPS | Vehicle | -- | 10 | -- | PO | 1x (1 hr post LPS) |
| 10 | 8 | LPS | Compound 1 | 100 | 10 | 10 | PO | 1x (1 hr post LPS) |
| 11 | 8 | LPS | Compound 1 | 300 | 10 | 30 | PO | 1x (1 hr post LPS) |

BID dosing occurred at approximately 10 - 12 hr intervals
²QD dosing occurred at approximately 24 hr intervals
³The doses of test item to be administered were calculated daily in mg/kg based on the latest body weight of the animal.
*On day 0, animals were dosed with test article 1 hour prior to LPS**On day 0, animals were dosed with test article 1 hour prior to LPS, and again 8 hours after the first dose

### Body Weight Measurement

The mice were weighed for randomization on study day -2 and were weighed again on day 0.

### Necropsy

The mice were euthanized for necropsy and sample collection on study day 0 at 6 hours post-LPS (groups 1-4 and 8) or 8 hours post-LPS (groups 9-11) or on study day 1 at 24 hours post-LPS (groups 5-7). At necropsy, the mice were bled to exsanguination by cardiac puncture followed by cervical dislocation. Whole blood was kept on wet ice and processed to plasma (K3EDTA, ≥200 µL/mouse), which was frozen on dry ice and stored at -80° C.

After euthanasia, a 20 G blunt-tipped needle was inserted into the trachea, and the lungs were washed 3 times with 0.8 mL of sterile saline. The samples were pooled into a 15-mL flip-top conical tube and kept on ice. The BAL was spun at 400 g for 5 minutes at 4° C., and supernatant was aspirated and aliquoted (≥100 µL/mouse for Luminex, ≥100 µL/mouse for protein assay, and remainder). The cell pellet was resuspended in 1 mL sterile saline and counted via Luna-FLTM. Absolute cell counts are included in Table 3. After the cell count, 100 µL of the cell suspension was spun down in a Cytospin® collection funnel with an attached slide. The slides were fixed followed by staining with a Diff-Quick stain. The cell differentiation counts were quantified under a light microscope.

Following lavage, whole lungs were infused with approximately 0.8 mL of neutral buffered formalin (NBF) via the trachea then excised and placed in 10% NBF at room temperature for histology.

### Cell Differentials

Stained Cytospin® slides were evaluated for cell differentials (lymphocytes, monocytes, neutrophils, eosinophils, and basophils) by microscopic evaluation using a field at 400x. A total of 100 cells (or the total number if less than 100) was counted, and the total percentage of each cell type and the number of cells per milliliter of BAL fluid were calculated.

### Cytokine Analysis Methods

BAL supernatant was analyzed by Luminex 200TM using a Milliplex® MAP Mouse Cytokine/Chemokine Magnetic Bead Panel - Immunology Multiplex Assay (kit catalog No. MCYTOMAG-70K, lot No. 3754184) according to the kit instructions. The samples were analyzed for the following analytes: IL-6 and TNFα.

Assay Procedure:
1. Add 200 µL of Wash Buffer into each well of the plate. Seal and mix on a plate shaker for 10 minutes at room temperature (20-25° C.).
2. Decant Wash Buffer and remove the residual amount from all wells by inverting the plate and tapping it smartly onto absorbent towels several times.
3. Add 25 µL of each Standard or Control into the appropriate wells. Assay Buffer should be used for 0 pg/mL standard (Background).
4. Add 25 µL of Assay Buffer to the sample wells.
5. Add 25 µL of appropriate matrix solution to the background, standards, and control wells.

When assaying serum or plasma, use the Serum Matrix provided in the kit. When assaying tissue culture or other supernatant, use proper control culture medium as the matrix solution.
6. Add 25 µL of Sample (diluted one part serum or plasma to one part Assay Buffer) into the appropriate wells.
7. Vortex Mixing Bottle and add 25 µL of the Mixed or Premixed Beads to each well. (Note: During addition of Beads, shake bead bottle intermittently to avoid settling.)
8. Seal the plate with a plate sealer. Wrap the plate with foil and incubate with agitation on a plate shaker overnight at 2-8° C.
9. Gently remove well contents and wash plate 2 times following kit instructions.
10. Add 25 µL of Detection Antibodies into each well. (Note: Allow the Detection Antibodies to warm to room temperature prior to addition.)
11. Seal, cover with foil and incubate with agitation on a plate shaker for 1 hour at room temperature (20-25° C.). Do not aspirate after incubation.
12. Add 25 µL Streptavidin-Phycoerythrin to each well containing the 25 µL of Detection Antibodies.
13. Seal, cover with foil and incubate with agitation on a plate shaker for 30 minutes at room temperature (20-25° C.).
14. Gently remove well contents and wash plate 2 times following kit instructions.
15. Add 150 µL of Sheath Fluid to all wells. Resuspend the beads on a plate shaker for 5 minutes.
16. Run plate on Luminex 200™.

### Protein Assay

BAL supernatant was analyzed for protein concentration using a PierceTM BCA Protein Assay Kit (ThermoFisher Scientific, catalogue No. 23225)

Assay Procedure:
1. Pipette 25 µL of each standard or unknown sample replicate into a microplate well (working range = 20-2000 µg/mL).
2. Add 200 µL of the BCA Working Reagent (WR) to each well and mix plate thoroughly on a plate shaker for 30 seconds.
3. Cover plate and incubate at 37° C. for 30 minutes.
4. Cool plate to room temperature. Measure the absorbance at or near 562 nm on a plate reader. Subtract the average 562 nm absorbance measurement of the Blank standard replicates from the 562 nm measurements of all other individual standard and unknown sample replicates.

### Morphologic Pathology Methods

Lungs are collected into formalin, routinely processed, sectioned longitudinally for maximum area visualization, and stained with hematoxylin and eosin (H&E). The entire section of lung is evaluated by light microscopy by a board-certified pathologist.

Peribronchiolar/perivascular Lymphoid Inflammation (BALT): The widths of lymphoid cell infiltrates of five of the most severely-affected areas are measured, and a mean is determined.

Interstitial Inflammation Score: The approximate percentage of lung affected by inflammation is evaluated and scored as follows:
0 = None
0.5 = Very minimal, ≤ 5%
1 = Minimal, 6-10%
2 = Mild, 11-25%
3 = Moderate, 26-50%
4 = Marked, 51-75%
5 = Severe, > 75%

Neutrophil Percent and Neutrophil Score: Inflammatory cell infiltrates are evaluated for approximate percent of neutrophils in the total infiltrate, rounded to 0, 10, 25, 50, or 75 percent. This value is then multiplied by the inflammation score to determine the neutrophil score.

Peribronchiolar/perivascular Edema Score: Peribronchiolar/perivascular widening by edema of five of the most severely-affected areas are measured, and a mean is determined. A score is assigned based on that mean as follows:
0 = None
0.5 = Very minimal, ≤ 25 microns
1 = Minimal, 26-50 microns
2 = Mild, 51-75 microns
3 = Moderate, 76-100 microns
4 = Marked, 101-150 microns
5 = Severe, > 150 microns

Airway Epithelial Damage Score: Lungs are scored for the degree of airway epithelial damage, according to the worst lesion present, as follows:
0 = None; normal intact airway epithelium
1 = Elongation and distortion of the cuboidal/columnar epithelial cells lining the airways
2 = Elongation associated with in folding of the epithelium and narrowing of the airway lumen
3 = Loss of epithelial cells resulting in broken airways

Histopathology Sum: A sum of all the above scores is calculated.

Goblet Cell hyperplasia Score: The approximate percentage of large and medium airways affected by goblet cell hyperplasia (as indicated by positive PAS/Alcian blue staining) is determined, and a score for each is assigned, as follows:
0 = None
0.5 = Very minimal, only one or two foci present
1 = Minimal, 1-10% of airway epithelium affected
2 = Mild, 11-25% of airway epithelium affected
3 = Moderate, 26-50% of airway epithelium affected
4 = Marked, 51-75% of airway epithelium affected
5 = Severe, > 75% of airway epithelium affected

### Results

### Body Weight Change from Day -2 (g)

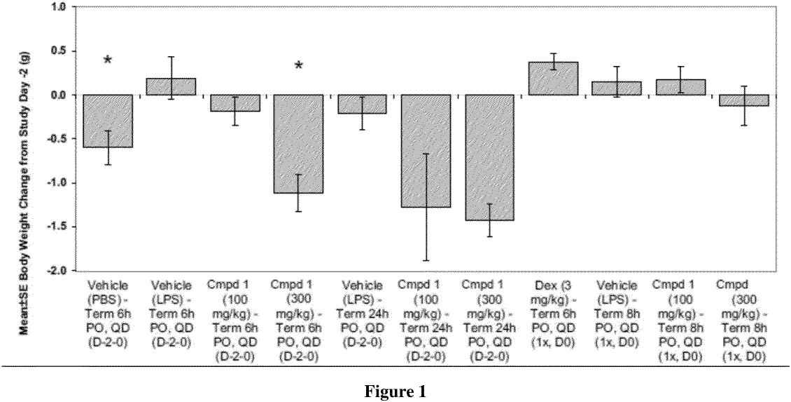

Vehicle disease control mice had mean weight gain of 0.59% at 6 hours post-LPS and 0.43% at 8 hours post-LPS on day 0 and mean weight loss of 0.60% at 24 hours post-LPS on day 1. Normal (PBS) control animals had mean weight loss of 1.58% at 6 hours post-LPS, which was a significant reduction compared to vehicle disease controls at the same time point. Mice treated with 300 mg/kg Compound 1 also had significantly increased body weight loss for animals necropsied at the 6 hour post-LPS timepoint as compared to the vehicle disease control group. No other statistically significant differences in body weight change were observed (FIG. 1).

### Lung Lavage Cell Differentials (Cells/mL)

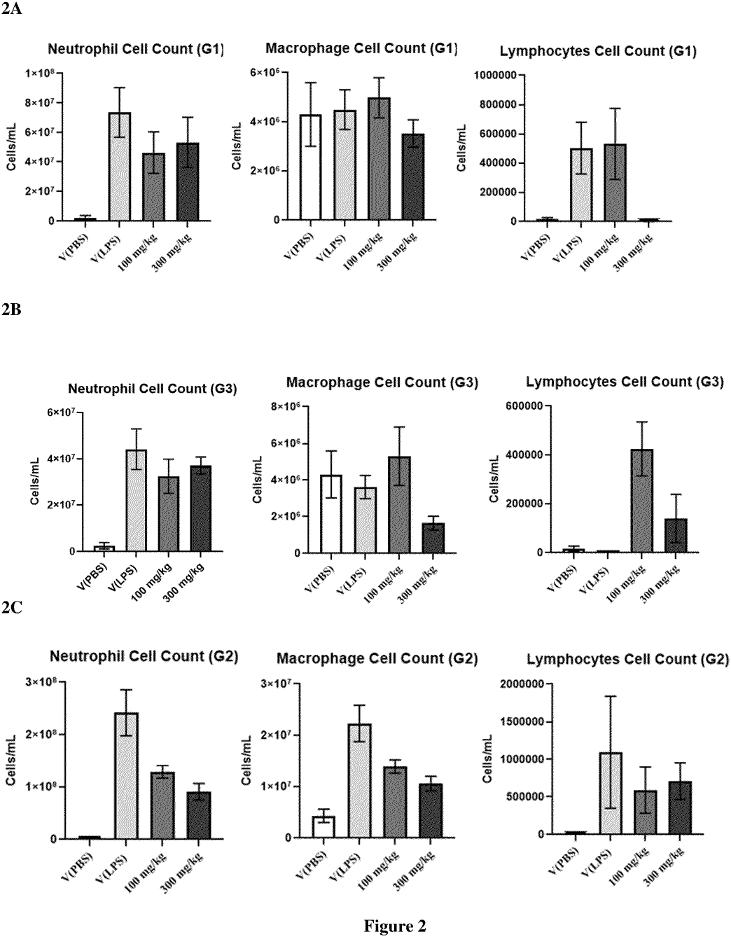

Neutrophils and lymphocytes per milliliter in BAL fluids were statistically increased in vehicle disease control mice at 6 hours post-LPS compared to the PBS control group (FIG. 2A). Mice treated with 100 mg/kg Compound 1 had significantly increased lymphocytes per milliliter compared to vehicle disease control mice at 8 hours post-LPS (FIG. 2B). At 24 hours post-LPS, neutrophils and macrophages per milliliter were significantly reduced in BAL fluids from mice treated with 100 or 300 mg/kg Compound 1 as compared to vehicle disease control mice (FIG. 2C). Mice treated with dexamethasone had significantly reduced neutrophils and lymphocytes per milliliter at 6 hours post-LPS compared to vehicle disease control animals.

### Absolute Total Nucleated Cells in Lung Lavage

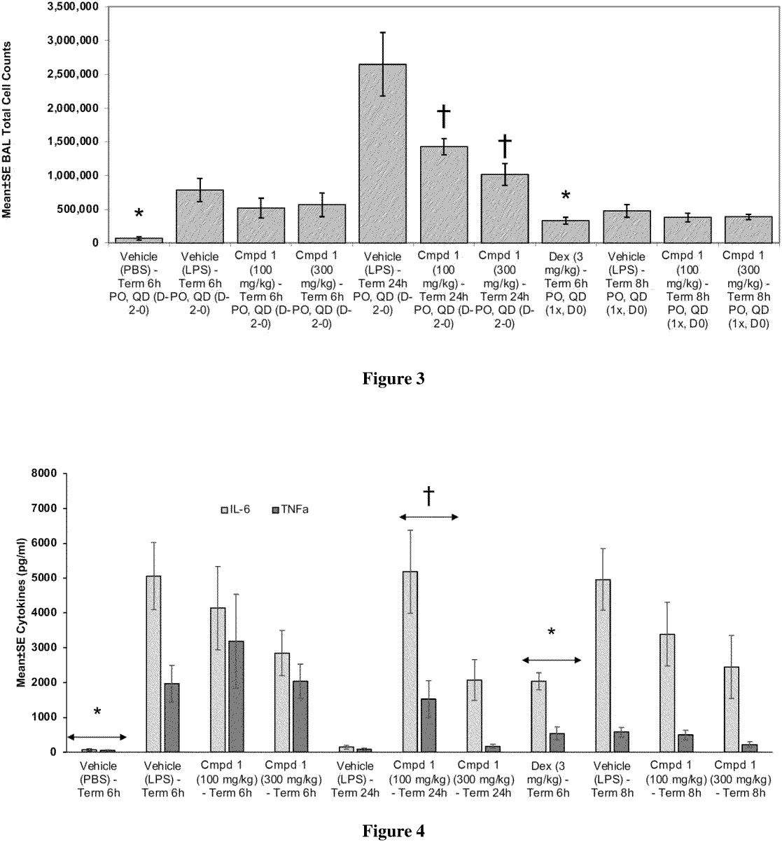

Mean total nucleated cells in BAL fluids were significantly increased in vehicle disease control mice at 6 hours post-LPS as compared to PBS control mice. Total nucleated cell counts were significantly reduced at 24 hours post-LPS in mice treated with 100 or 300 mg/kg Compound 1 as compared to the vehicle disease control group. Mice treated with dexamethasone had significantly reduced total nucleated cell counts at 6 hours post-LPS as compared to vehicle disease control mice (FIG. 3).

### Luminex Cytokine Levels in BAL: IL-6 and TNFα

Vehicle disease control mice had significantly increased IL-6 and TNFαlevels in BAL fluids at 6 hours post-LPS compared to the PBS control group. Mice treated with 100 mg/kg and 300 mg/kg Compound 1 had a decrease in IL-6 levels at 6 hours post-LPS compared to vehicle disease control, though not statistically significant. Mice treated with 100 mg/kg Compound 1 had significantly increased IL-6 and TNFαlevels as compared vehicle disease control mice at 24 hours post-LPS. Mice treated with dexamethasone had significantly reduced IL-6 and TNFα levels at 6 hours post-LPS compared to vehicle disease control mice. Mice treated with 100 mg/kg and 300 mg/kg Compound 1 had a decrease in IL-6 and TNFαlevels at 8 hours post-LPS compared to vehicle disease control, though not statistically significant (FIG. 4).

### Peribronchiolar lymphoid Inflammation Width

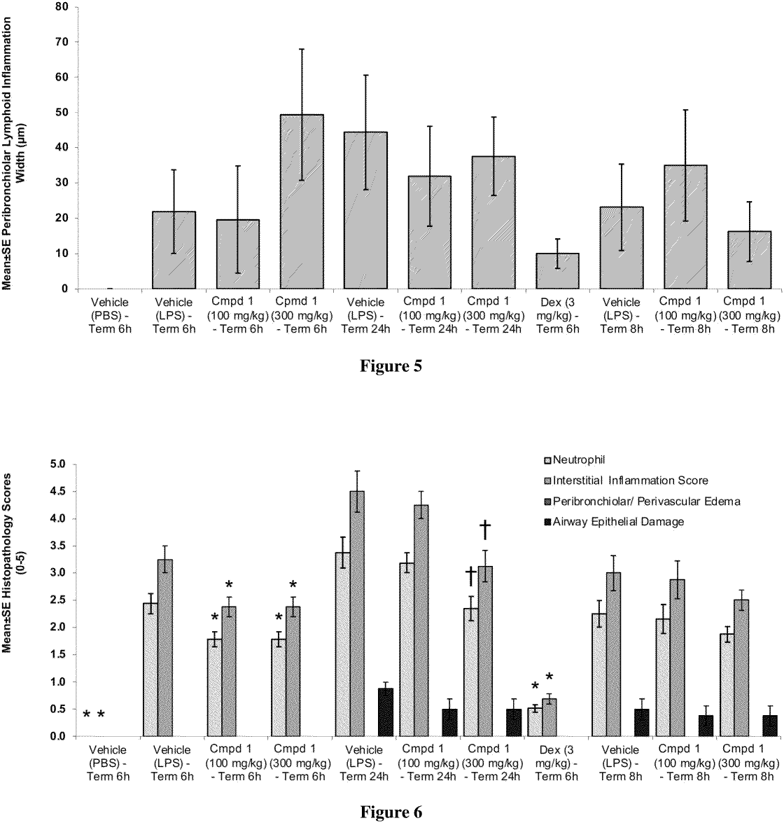

Normal (PBS) control mice had no microscopic evidence of peribronchiolar lymphoid inflammation. Vehicle disease control animals had peribronchiolar lymphoid inflammation widths that peaked 24 hours post-LPS (mean width of 44.4 µm) although peribronchiolar lymphoid inflammation widths at 6 hours post-LPS did not differ statistically from the PBS control group. Slight decrease in peribronchiolar lymphoid inflammation widths at 24 hours post-LPS for mice treated with Compound 1 as compared to vehicle disease control mice, although statistically insignificant (FIG. 5).

### Lung Inflammation Scores

At 6 hours post-LPS, vehicle disease control mice had interstitial inflammation of the lungs that ranged from mild to marked and neutrophils comprised 75% of total cell infiltrates. No peribronchiolar/perivascular edema or airway epithelial damage was observed at 6 hours post-LPS. Neutrophil and interstitial inflammation scores were statistically increased in the vehicle disease control group at 6 hours post-LPS as compared to the PBS control group. At 8 hours post-LPS, vehicle disease control mice had neutrophil and interstitial inflammation scores that were generally similar to the 6-hour disease control group along with distortion/elongation of the cuboidal/columnar epithelial cells lining the airways in 3 of 8 mice. At 24 hours post-LPS, vehicle disease control mice had increased neutrophil and interstitial inflammation scores compared to earlier time points with distortion/elongation of epithelial cells seen in 7 of 8 mice. No peribronchiolar/perivascular edema was observed at 8 or 24 hours post-LPS (FIG. 6).

Neutrophil and interstitial inflammation scores were significantly reduced at 6 hours post-LPS in mice treated with 100 mg/kg Compound 1 (27% reductions), 300 mg/kg Compound 1 (27% reductions), or dexamethasone (79% reductions) and at 24 hours post-LPS in mice treated with 300 mg/kg Compound 1 (31 % reductions) as compared to the respective 6-hour and 24-hour vehicle disease control groups (FIG. 6).

### Lung Summed Inflammation Scores

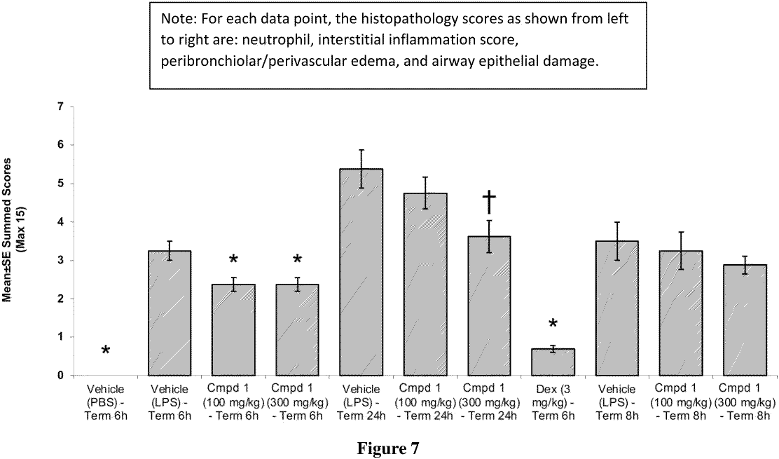

Summed lung scores were significantly reduced at 6 hours post-LPS in mice treated with 100 mg/kg Compound 1 (27% reduction), 300 mg/kg Compound 1 (27%), or dexamethasone (79%) and at 24 hours post-LPS in mice treated with 300 mg/kg Compound 1 (33%) as compared to the respective 6-hour and 24-hour vehicle disease control groups (FIG. 7).

### Conclusions

### Necropsy: 6 Hours Post-LPS

Male Swiss Webster mice with LPS-induced pulmonary inflammation that were treated PO, QD with Compound 1 (100 mg/kg; final dose 1 h pre-LPS for a total of 3 doses) and then terminated 6 hours post-LPS injection had absolute body weight changes, total nucleated cell counts, cell differentials, and cytokine concentrations (IL-6 and TNFα) in BAL fluids that did not differ statistically from the disease control group. Results of treatment at 300 mg/kg were generally similar although this higher dose resulted in statistically significant absolute weight loss for animals necropsied at the 6 hour time point as compared to the vehicle disease control group. Histopathologic evaluation of lungs showed statistically significant (27%) reductions in interstitial inflammation, neutrophil, and summed lung scores for mice treated with Compound 1 at both doses as compared to the vehicle disease control group.

Mice treated with the positive control dexamethasone (3 mg/kg; PO: dosed 1 h pre-LPS) and terminated 6 hours post-LPS had absolute body weight changes that did not differ statistically from the vehicle disease control group. Total nucleated cell counts were statistically reduced in this group (63% reduction; 24% reduction in viable cells) compared to vehicle disease controls, and evaluation of cell differentials showed that dexamethasone-treated mice had significantly reduced absolute and relative neutrophil and lymphocytes and significantly increased relative macrophages. Evaluation of cytokine levels in BAL fluid showed significant reductions in IL-6 and TNFαconcentration for mice treated with dexamethasone compared to the vehicle disease control group, and lung histopathology showed significant reductions (79%) in interstitial inflammation, neutrophil, and summed lung scores.

### Necropsy: 8 Hours Post-LPS

Mice treated with a single dose of Compound 1 (100 or 300 mg/kg; PO: dosed 1 h post-LPS on day 0) that were terminated at 8 hours post-LPS showed no significant beneficial effects on disease parameters compared to vehicle disease control mice. Evaluation of cell differentials revealed significant increases in absolute and relative lymphocyte counts in the 100 mg/kg group with no significant differences observed in absolute body weight change, total nucleated cell counts, BAL cytokine concentrations, or lung histopathology.

### Necropsy: 24 Hours Post-LPS

Mice treated PO, QD with Compound 1 (100 or 300 mg/kg; final dose 7 h post-LPS on day 0 for a total of 4 doses) had absolute body weight changes that did not differ statistically from the vehicle disease control group. Total nucleated cell counts were significantly reduced at 24 hours post-LPS in mice treated with 100 or 300 mg/kg Compound 1 (47% and 63% reductions, respectively) with correspondingly significant reductions in absolute neutrophil (47% and 63%, respectively) and macrophage (47% and 65%, respectively) concentrations (cells/mL). Evaluation of BAL cytokine concentrations showed significant increases in IL-6 and TNFαfor mice dosed at 100 mg/kg compared to the vehicle disease control group. Histopathologic evaluation of lungs revealed significant reductions in interstitial inflammation (31% reduction), neutrophil scores (31%), and summed lung scores (33%) for mice treated at 300 mg/kg compared to the vehicle disease controls.

### Summary of Conclusion

Daily oral treatment with Compound 1 (100 or 300 mg/kg) resulted in beneficial effects on LPS-induced lung inflammation in male Swiss Webster mice as determined by significant reductions in total nucleated cell counts and significant changes in cell differentials at 24 hours post-LPS as well as significant improvements in lung histopathology scores at 6 and 24 hours post-LPS. Dosing at 100 mg/kg also resulted in significant increases in body weight loss at 6 hours post-LPS and significant increases in BAL cytokine concentrations (IL-6 and TNFα) at 24 hours post-LPS compared to disease controls. Results of treatment at 8 hours post-LPS following a single dose of Compound 1 (100 or 300 mg/kg) administered 1 hour post-LPS were generally not significant. Results of treatment with dexamethasone (3 mg/kg, PO, 1x: 1 hour pre-LPS) at 6 hours post-LPS were as expected in that treatment significantly ameliorated disease severity.

### FIRST SET OF CERTAIN EXEMPLARY EMBODIMENTS

Additional exemplary embodiments are described in the following numbered paragraphs 1-435 of this first set of exemplary embodiments.

1. A method of treating a respiratory viral infection, comprising administering to a patient in need thereof a compound that is a HIF prolyl hydroxylase inhibitor or a HIF-alpha stabilizer, or a pharmaceutically acceptable salt thereof.

2. The method of paragraph 1, wherein the respiratory viral infection is induced by or associated with a virus that could lead to viral pneumonia.

3. The method of paragraph 1 or 2, wherein the respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus.

4. The method of any one of paragraphs 1-3, wherein the respiratory viral infection is induced by or associated with a coronavirus.

5. The method of paragraph 4, wherein the coronavirus is the SARS-CoV, MERS-CoV, or SARS-CoV-2.

6. The method of any one of paragraphs 1-5, wherein the respiratory viral infection is lower respiratory infection.

7. The method of any one of paragraphs 1-6, wherein the respiratory viral infection is pulmonary viral infection.

8. The method of any one of paragraphs 1-5, wherein the respiratory viral infection is upper respiratory infection.

9. The method of any one of paragraphs 1-8, wherein the respiratory viral infection is COVID-19.

10. The method of any one of paragraphs 1-9, wherein the respiratory viral infection induces a lung disease.

11. The method of paragraph 10, wherein the lung disease is selected from acute lung injury (ALI), bronchitis, pneumonia, pulmonary fibrosis, asthma, or acute respiratory distress syndrome (ARDS).

12. The method of paragraph 10 or 11, wherein the patient develops pulmonary hypertension.

13. The method of paragraph 10 or 11, wherein the patient develops multi-organ failure.

14. The method of paragraph 13, wherein the multi-organ failure comprise heart failure, liver failure, lung failure kidney failure, and gastrointestinal (GI) system failure.

15. A method of treating a pulmonary viral infection, comprising administering to a patient in need thereof a compound that is a HIF prolyl hydroxylase inhibitor or a HIF-alpha stabilizer, or a pharmaceutically acceptable salt thereof.

16. The method of paragraph 15, wherein the pulmonary viral infection is induced by or associated with a coronavirus, an influenza A, an influenza B, a Rhinovirus, a respiratory syncytial virus (RSV), an adenovirus, a human parainfluenza virus (HPIV) or an enterovirus.

17. The method of paragraph 15 or 16, wherein the pulmonary viral infection is induced by or associated with a coronavirus.

18. The method of paragraph 17, wherein the coronavirus is the SARS-CoV, MERS-CoV, or SARS-CoV-2.

19. The method of any one of paragraphs 15-18, wherein the pulmonary viral infection is COVID-19.

20. The method of any one of paragraphs 15-19, wherein the pulmonary viral infection induces a lung disease.

21. The method of paragraph 20, wherein the lung disease is selected from acute lung injury (ALI), bronchitis, pneumonia, pulmonary fibrosis, asthma, or acute respiratory distress syndrome (ARDS).

22. The method of paragraph 20 or 21, wherein the patient develops pulmonary hypertension.

23. The method of paragraph 20 or 21, wherein the patient develops multi-organ failure.

24. The method of paragraph 23, wherein the multi-organ failure comprises heart failure, liver failure, lung failure, kidney failure and gastrointestinal (GI) system failure.

25. A method of treating a coronavirus infection, comprising administering to a patient in need thereof a compound that is a HIF prolyl hydroxylase inhibitor or a HIF-alpha stabilizer, or a pharmaceutically acceptable salt thereof.

26. A method of treating or preventing a lung disease that is acute lung injury (ALI) in a patient in need thereof, comprising administering to the patient a compound that is a HIF prolyl hydroxylase inhibitor or a HIF-alpha stabilizer, or a pharmaceutically acceptable salt thereof.

27. The method of paragraph 26, wherein said patient has a viral infection, sepsis, pneumonia, aspiration, trauma, pancreatitis, blood transfusion, and/or smoke or toxic gas inhalation.

28. The method of paragraph 26 or 27, wherein said acute lung injury (ALI) is induced by or associated with a viral infection, sepsis, pneumonia, aspiration, trauma, pancreatitis, blood transfusion, and/or smoke or toxic gas inhalation.

29. The method of any one of paragraphs 26-28, wherein said patient is mechanically ventilated.

30. A method of treating or preventing a lung disease that is acute lung injury (ALI) in a patient in need thereof, comprising administering to the patient a compound that is a HIF prolyl hydroxylase inhibitor or a HIF-alpha stabilizer, or a pharmaceutically acceptable salt thereof, and

wherein said ALI is induced by or associated with a ventilator.

31. The method of paragraph 30, wherein said patient has a viral infection, sepsis, pneumonia, aspiration, trauma, pancreatitis, blood transfusion, and/or smoke or toxic gas inhalation.

32. A method of treating or preventing a lung disease that is acute lung injury (ALI) in a patient having a respiratory viral infection, comprising administering to the patient a compound that is a HIF prolyl hydroxylase inhibitor or a HIF-alpha stabilizer, or a pharmaceutically acceptable salt thereof.

33. A method of treating or preventing a lung disease that is acute respiratory distress syndrome (ARDS) in a patient having a respiratory viral infection, comprising administering to the patient a compound that is a HIF prolyl hydroxylase inhibitor or a HIF-alpha stabilizer, or a pharmaceutically acceptable salt thereof.

34. The method of paragraph 32 or 33, wherein the respiratory viral infection is infection by a coronavirus, an influenza A, an influenza B, a Rhinovirus, a respiratory syncytial virus (RSV), an adenovirus, a human parainfluenza virus (HPIV) or an enterovirus.

35. The method of any one of paragraphs 32-34, wherein the respiratory viral infection is infection by a coronavirus.

36. The method of paragraph 35, wherein the coronavirus is the SARS-CoV, MERS-CoV, or SARS-CoV-2.

37. The method of any one of paragraphs 32-36, wherein the respiratory viral infection is COVID-19.

38. The method of paragraph 32, wherein the patient with acute lung injury develops acute respiratory distress syndrome (ARDS).

39. The method of any one of paragraphs 32-38, wherein the patient with ALI or ARDS develops pulmonary hypertension.

40. The method of any one of paragraphs 32-38, wherein the patient with ALI or ARDS develops multi-organ failure.

41. The method of paragraph 40, wherein the multi-organ failure comprise heart failure, liver failure, lung failure kidney failure, and gastrointestinal (GI) system failure.

42. The method of any one of paragraphs 32-41, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

43. The method of any one of paragraphs 32-41, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or any combination thereof.

44. The method of any one of paragraphs 32-43, wherein ALI or ARDS is induced by or associated with the viral infection.

45. A method of treating or preventing a lung disease that is acute lung injury (ALI) in a patient having a pulmonary viral infection, comprising administering to the patient a compound that is a HIF prolyl hydroxylase inhibitor or a HIF-alpha stabilizer, or a pharmaceutically acceptable salt thereof.

46. A method of treating or preventing a lung disease that is acute respiratory distress syndrome (ARDS) in a patient having a pulmonary viral infection, comprising administering to the patient a compound that is a HIF prolyl hydroxylase inhibitor or a HIF-alpha stabilizer, or a pharmaceutically acceptable salt thereof.

47. The method of paragraph 45 or 46, wherein the pulmonary viral infection is infection by a coronavirus, an influenza A, an influenza B, a Rhinovirus, a respiratory syncytial virus (RSV), an adenovirus, a human parainfluenza virus (HPIV) or an enterovirus.

48. The method of any one of paragraphs 45-47, wherein the pulmonary viral infection is infection by a coronavirus.

49. The method of paragraph 48, wherein the coronavirus is the SARS-CoV, MERS-CoV, or SARS-CoV-2.

50. The method of any one of paragraphs 45-49, wherein the pulmonary viral infection is COVID-19.

51. The method of paragraph 45, wherein the patient with acute lung injury develops acute respiratory distress syndrome (ARDS).

52. The method of any one of paragraphs 45-51, wherein the patient with ALI or ARDS develops pulmonary hypertension.

53. The method of any one of paragraphs 45-51, wherein the patient with ALI or ARDS develops multi-organ failure.

54. The method of paragraph 53, wherein the multi-organ failure comprise heart failure, liver failure, lung failure kidney failure, and gastrointestinal (GI) system failure.

55. The method of any one of paragraphs 45-54, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

56. The method of any one of paragraphs 45-54, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or any combination thereof.

57. The method of any one of paragraphs 45-56, wherein ALI or ARDS is induced by or associated with the viral infection.

58. A method of treating or preventing a lung disease that is acute lung injury (ALI) in a patient infected with a coronavirus, comprising administering to the patient a compound that is a HIF prolyl hydroxylase inhibitor or a HIF-alpha stabilizer, or a pharmaceutically acceptable salt thereof.

59. A method of treating or preventing a lung disease that is acute respiratory distress syndrome (ARDS) in a patient infected with a coronavirus, comprising administering to the patient a compound that is a HIF prolyl hydroxylase inhibitor or a HIF-alpha stabilizer, or a pharmaceutically acceptable salt thereof.

60. The method of paragraph 58 or 59, wherein the coronavirus is the SARS-CoV, MERS-CoV, or SARS-CoV-2.

61. The method of any one of paragraphs 58-60, wherein the patient is infected with SARS-CoV-2.

62. The method of any one of paragraphs 58-61, wherein the patient with ALI or ARDS develops pulmonary hypertension.

63. The method of any one of paragraphs 58-61, wherein the patient with ALI or ARDS develops multi-organ failure.

64. The method of paragraph 63, wherein the multi-organ failure comprise heart failure, liver failure, lung failure kidney failure, and gastrointestinal (GI) system failure.

65. The method of any one of paragraphs 58-64, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

66. The method of any one of paragraphs 58-64, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or any combination thereof.

67. The method of any one of paragraphs 58-66, wherein ALI or ARDS is induced by or associated with the viral infection.

68. The method of any one of paragraphs 1-67, wherein said patient is an adult.

69. The method of any one of paragraphs 1-68, wherein said patient exhibits one or more symptoms of hypoxia.

70. The method of paragraph 69, wherein the one or more symptoms of hypoxia include changes in the color of the skin, confusion, cough, fast heart rate, rapid breathing, shortness of breath, sweating, and/or wheezing.

71. The method of paragraph 69, wherein the patient has a low oxygen saturation of hemoglobin.

72. The method of any one of paragraphs 1-71, wherein the patient has an oxygen saturation of hemoglobin that is about 94% or less.

73. A method of reducing mortality and morbidity related to acute lung injury (ALI) or acute respiratory distress syndrome (ARDS) in a patient having a respiratory viral infection, comprising administering to a patient in need thereof a compound that is a HIF prolyl hydroxylase inhibitor or a HIF-alpha stabilizer, or a pharmaceutically acceptable salt thereof.

74. The method of paragraph 73, wherein the patient has a lower respiratory infection.

75. The method of paragraph 73, wherein the patient has an upper respiratory infection.

76. A method of reducing mortality and morbidity related to acute lung injury (ALI) or acute respiratory distress syndrome (ARDS) in a patient having a pulmonary viral infection, comprising administering to a patient in need thereof a compound that is a HIF prolyl hydroxylase inhibitor or a HIF-alpha stabilizer, or a pharmaceutically acceptable salt thereof.

77. The method of any one of paragraphs 73-76, wherein the viral infection is induced by or associated with a virus that could lead to viral pneumonia.

78. The method of any one of paragraphs 73-77, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

79. A method of reducing mortality and morbidity related to acute lung injury (ALI) or acute respiratory distress syndrome (ARDS) in a patient infected with a coronavirus, comprising administering to a patient in need thereof a compound that is a HIF prolyl hydroxylase inhibitor or a HIF-alpha stabilizer, or a pharmaceutically acceptable salt thereof.

80. The method of any one of paragraphs 73-79, wherein the acute lung injury (ALI) or acute respiratory distress syndrome (ARDS) is induced by or associated with the virus.

81. The method of any one of paragraphs 73-80, wherein the patient is infected by a coronavirus that is SARS-CoV, MERS-CoV, or SARS-CoV-2.

82. The method of any one of paragraphs 73-81, wherein the patient has COVID-19.

83. The method of any one of paragraphs 73-82, wherein the patient is receiving mechanical ventilation.

84. The method of any one of paragraphs 73-83, wherein the patient has ventilator induced ALI.

85. A method of reducing incidence, severity, or risk of acute lung injury (ALI) or acute respiratory distress syndrome (ARDS) in a patient having a respiratory viral infection, comprising administering to a patient in need thereof a compound that is a HIF prolyl hydroxylase inhibitor or a HIF-alpha stabilizer, or a pharmaceutically acceptable salt thereof.

86. The method of paragraph 85, wherein the patient has a lower respiratory infection.

87. The method of paragraph 85, wherein the patient has an upper respiratory infection.

88. A method of reducing incidence, severity, or risk of acute lung injury (ALI) or acute respiratory distress syndrome (ARDS) in a patient having a pulmonary viral infection, comprising administering to a patient in need thereof a compound that is a HIF prolyl hydroxylase inhibitor or a HIF-alpha stabilizer, or a pharmaceutically acceptable salt thereof.

89. The method of any one of paragraphs 85-88, wherein the viral infection is induced by or associated with a virus that could lead to viral pneumonia.

90. The method of any one of paragraphs 85-89, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

91. A method of reducing incidence, severity, or risk of acute lung injury (ALI) or acute respiratory distress syndrome (ARDS) in a patient infected with a coronavirus, comprising administering to a patient in need thereof a compound that is a HIF prolyl hydroxylase inhibitor or a HIF-alpha stabilizer, or a pharmaceutically acceptable salt thereof.

92. The method of any one of paragraphs 85-91, wherein the acute lung injury (ALI) or acute respiratory distress syndrome (ARDS) is induced by or associated with the virus.

93. The method of any one of paragraphs 85-92, wherein the patient is infected by a coronavirus that is SARS-CoV, MERS-CoV, or SARS-CoV-2.

94. The method of any one of paragraphs 85-93, wherein the patient has COVID-19.

95. The method of any one of paragraphs 85-94, wherein the patient is receiving mechanical ventilation.

96. The method of any one of paragraphs 85-95, wherein the patient has ventilator induced ALI.

97. The method of any one of paragraphs 73-96, wherein said patient is an adult.

98. The method of any one of paragraphs 73-97, wherein said patient exhibits one or more symptoms of hypoxia.

99. The method of paragraph 98, wherein the patient has a lower oxygen saturation of hemoglobin compared to normal oxygen saturation.

100. The method of any one of paragraphs 73-99, wherein the patient has an oxygen saturation of hemoglobin that is about 94% or less.

101. A method of treating or preventing lung inflammation or pneumonia in a patient having a respiratory viral infection, comprising administering to a patient in need thereof a compound that is a HIF prolyl hydroxylase inhibitor or a HIF-alpha stabilizer, or a pharmaceutically acceptable salt thereof.

102. A method of reducing mortality and morbidity related to lung inflammation or pneumonia in a patient having a respiratory viral infection, comprising administering to a patient in need thereof a compound that is a HIF prolyl hydroxylase inhibitor or a HIF-alpha stabilizer, or a pharmaceutically acceptable salt thereof.

103. A method of reducing incidence, severity, or risk of lung inflammation or pneumonia in a patient having a respiratory viral infection, comprising administering to a patient in need thereof a compound that is a HIF prolyl hydroxylase inhibitor or a HIF-alpha stabilizer, or a pharmaceutically acceptable salt thereof.

104. The method of any one of paragraphs 101-103, wherein the patient has a lower respiratory infection.

105. The method of any one of paragraphs 101-103, wherein the patient has an upper respiratory infection.

106. A method of treating or preventing lung inflammation or pneumonia in a patient having a pulmonary viral infection, comprising administering to a patient in need thereof a compound that is a HIF prolyl hydroxylase inhibitor or a HIF-alpha stabilizer, or a pharmaceutically acceptable salt thereof.

107. A method of reducing mortality and morbidity related to lung inflammation or pneumonia in a patient having a pulmonary viral infection, comprising administering to a patient in need thereof a compound that is a HIF prolyl hydroxylase inhibitor or a HIF-alpha stabilizer, or a pharmaceutically acceptable salt thereof.

108. A method of reducing incidence, severity, or risk of lung inflammation or pneumonia in a patient having a pulmonary viral infection, comprising administering to a patient in need thereof a compound that is a HIF prolyl hydroxylase inhibitor or a HIF-alpha stabilizer, or a pharmaceutically acceptable salt thereof.

109. The method of any one of paragraphs 101-108, wherein the viral infection is induced by or associated with a virus that could lead to viral pneumonia.

110. The method of any one of paragraphs 101-109, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

111. A method of treating or preventing lung inflammation or pneumonia in a patient infected with a coronavirus, comprising administering to a patient in need thereof a compound that is a HIF prolyl hydroxylase inhibitor or a HIF-alpha stabilizer, or a pharmaceutically acceptable salt thereof.

112. A method of reducing mortality and morbidity related to lung inflammation or pneumonia in a patient infected with a coronavirus, comprising administering to a patient in need thereof a compound that is a HIF prolyl hydroxylase inhibitor or a HIF-alpha stabilizer, or a pharmaceutically acceptable salt thereof.

113. A method of reducing incidence, severity, or risk of lung inflammation or pneumonia in a patient infected with a coronavirus, comprising administering to a patient in need thereof a compound that is a HIF prolyl hydroxylase inhibitor or a HIF-alpha stabilizer, or a pharmaceutically acceptable salt thereof.

114. The method of any one of paragraphs 101-113, wherein the patient is infected by a coronavirus that is SARS-CoV, MERS-CoV, or SARS-CoV-2.

115. The method of any one of paragraphs 101-114, wherein the patient has COVID-19.

116. The method of any one of paragraphs 101-115, wherein the lung inflammation or pneumonia is induced by or associated with the viral infection.

117. The method of aragraph 116, wherein the lung inflammation or pneumonia is induced by or associated with COVID-19.

118. A method of treating or preventing COVID-19 related pneumonia, comprising administering to a patient in need thereof a compound that is a HIF prolyl hydroxylase inhibitor or a HIF-alpha stabilizer, or a pharmaceutically acceptable salt thereof, and wherein the patient has acute lung injury(ALI), acute respiratory distress syndrome (ARDS), and/or organ failure.

119. The method of paragraph 118, wherein any of the acute lung injury (ALI), acute respiratory distress syndrome (ARDS), and/or organ failure is COVID-19 related.

120. A method of treating or preventing COVID-19 related acute lung injury (ALI) in a patient having pneumonia, comprising administering to a patient in need thereof a compound that is a HIF prolyl hydroxylase inhibitor or a HIF-alpha stabilizer, or a pharmaceutically acceptable salt thereof.

121. A method of treating or preventing COVID-19 related acute respiratory distress syndrome (ARDS) in a patient having pneumonia, comprising administering to a patient in need thereof a compound that is a HIF prolyl hydroxylase inhibitor or a HIF-alpha stabilizer, or a pharmaceutically acceptable salt thereof.

122. A method of treating or preventing COVID-19 related organ failure, comprising administering to a patient in need thereof a compound that is a HIF prolyl hydroxylase inhibitor or a HIF-alpha stabilizer, or a pharmaceutically acceptable salt thereof.

123. The method of any one of paragraphs 101-122, wherein the patient is receiving mechanical ventilation.

124. A method of treating or preventing cardiac dysfunction in a patient having a respiratory viral infection, comprising administering to a patient in need thereof a compound that is a HIF prolyl hydroxylase inhibitor or a HIF-alpha stabilizer, or a pharmaceutically acceptable salt thereof.

125. A method of reducing mortality and morbidity related to an adverse cardiac event in a patient having a respiratory viral infection, comprising administering to a patient in need thereof a compound that is a HIF prolyl hydroxylase inhibitor or a HIF-alpha stabilizer, or a pharmaceutically acceptable salt thereof.

126. A method of reducing incidence, severity, or risk of an adverse cardiac event in a patient having a respiratory viral infection, comprising administering to a patient in need thereof a compound that is a HIF prolyl hydroxylase inhibitor or a HIF-alpha stabilizer, or a pharmaceutically acceptable salt thereof.

127. The method of any one of paragraphs 124-126, wherein the patient has a lower respiratory infection.

128. The method of any one of paragraphs 124-126, wherein the patient has an upper respiratory infection.

129. A method of treating or preventing cardiac dysfunction in a patient having a pulmonary viral infection, comprising administering to a patient in need thereof a compound that is a HIF prolyl hydroxylase inhibitor or a HIF-alpha stabilizer, or a pharmaceutically acceptable salt thereof.

130. A method of reducing mortality and morbidity related to an adverse cardiac event in a patient having a pulmonary viral infection, comprising administering to a patient in need thereof a compound that is a HIF prolyl hydroxylase inhibitor or a HIF-alpha stabilizer, or a pharmaceutically acceptable salt thereof.

131. A method of reducing incidence, severity, or risk of an adverse cardiac event in a patient having a pulmonary viral infection, comprising administering to a patient in need thereof a compound that is a HIF prolyl hydroxylase inhibitor or a HIF-alpha stabilizer, or a pharmaceutically acceptable salt thereof.

132. The method of any one of paragraphs 124-131, wherein the viral infection is induced by or associated with a virus that could lead to viral pneumonia.

133. The method of any one of paragraphs 124-132, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

134. A method of treating or preventing cardiac dysfunction in a patient infected with a coronavirus, comprising administering to a patient in need thereof a compound that is a HIF prolyl hydroxylase inhibitor or a HIF-alpha stabilizer, or a pharmaceutically acceptable salt thereof.

135. A method of reducing mortality and morbidity related to an adverse cardiac event in a patient infected with a coronavirus, comprising administering to a patient in need thereof a compound that is a HIF prolyl hydroxylase inhibitor or a HIF-alpha stabilizer, or a pharmaceutically acceptable salt thereof.

136. A method of reducing incidence, severity, or risk of an adverse cardiac event in a patient infected with a coronavirus, comprising administering to a patient in need thereof a compound that is a HIF prolyl hydroxylase inhibitor or a HIF-alpha stabilizer, or a pharmaceutically acceptable salt thereof.

137. The method of any one of paragraphs 124-136, wherein the patient is infected by a coronavirus that is SARS-CoV, MERS-CoV, or SARS-CoV-2.

138. The method of any one of paragraphs 124-137, wherein the patient has COVID-19.

139. The method of any one of paragraphs 124, 129, and 134, wherein the cardiac dysfunction is induced by or associated with the viral infection.

140. The method of paragraph 139, wherein the cardiac dysfunction is induced by or associated with COVID-19.

141. The method of any one of paragraphs 125-126, 130-131, and 135-136 wherein an adverse cardiac event is induced by or associated with the viral infection.

142. The method of paragraph 141, wherein an adverse cardiac event is induced by or associated with COVID-19.

143. The method of any one of paragraphs 124-142, wherein the patient is receiving mechanical ventilation.

144. A method of treating or preventing hypotension in a patient having a respiratory viral infection, comprising administering to a patient in need thereof a compound that is a HIF prolyl hydroxylase inhibitor or a HIF-alpha stabilizer, or a pharmaceutically acceptable salt thereof.

145. A method of reducing mortality and morbidity related to hypotension in a patient having a respiratory viral infection, comprising administering to a patient in need thereof a compound that is a HIF prolyl hydroxylase inhibitor or a HIF-alpha stabilizer, or a pharmaceutically acceptable salt thereof.

146. A method of reducing incidence, severity, or risk of hypotension in a patient having a respiratory viral infection, comprising administering to a patient in need thereof a compound that is a HIF prolyl hydroxylase inhibitor or a HIF-alpha stabilizer, or a pharmaceutically acceptable salt thereof.

147. The method of any one of paragraphs 144-146, wherein the patient has a lower respiratory infection.

148. The method of any one of paragraphs 144-146, wherein the patient has an upper respiratory infection.

149. A method of treating or preventing hypotension in a patient having a pulmonary viral infection, comprising administering to a patient in need thereof a compound that is a HIF prolyl hydroxylase inhibitor or a HIF-alpha stabilizer, or a pharmaceutically acceptable salt thereof.

150. A method of reducing mortality and morbidity related to hypotension in a patient having a pulmonary viral infection, comprising administering to a patient in need thereof a compound that is a HIF prolyl hydroxylase inhibitor or a HIF-alpha stabilizer, or a pharmaceutically acceptable salt thereof.

151. A method of reducing incidence, severity, or risk of hypotension in a patient having a pulmonary viral infection, comprising administering to a patient in need thereof a compound that is a HIF prolyl hydroxylase inhibitor or a HIF-alpha stabilizer, or a pharmaceutically acceptable salt thereof.

152. The method of any one of paragraphs 144-151, wherein the viral infection is induced by or associated with a virus that could lead to viral pneumonia.

153. The method of any one of paragraphs 144-152, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

154. A method of treating or preventing hypotension in a patient infected with a coronavirus, comprising administering to a patient in need thereof a compound that is a HIF prolyl hydroxylase inhibitor or a HIF-alpha stabilizer, or a pharmaceutically acceptable salt thereof.

155. A method of reducing mortality and morbidity related to hypotension in a patient infected with a coronavirus, comprising administering to a patient in need thereof a compound that is a HIF prolyl hydroxylase inhibitor or a HIF-alpha stabilizer, or a pharmaceutically acceptable salt thereof.

156. A method of reducing incidence, severity, or risk of hypotension in a patient infected with a coronavirus, comprising administering to a patient in need thereof a compound that is a HIF prolyl hydroxylase inhibitor or a HIF-alpha stabilizer, or a pharmaceutically acceptable salt thereof.

157. The method of any one of paragraphs 144-156, wherein the patient is infected by a coronavirus that is SARS-CoV, MERS-CoV, or SARS-CoV-2.

158. The method of any one of paragraphs 144-157, wherein the patient has COVID-19.

159. The method of any one of paragraphs 144-158, wherein the hypotension is induced by or associated with the viral infection.

160. The method of paragraph 159, wherein the hypotension is induced by or associated with COVID-19.

161. The method of any one of paragraphs 144-160, wherein the patient is receiving mechanical ventilation.

162. A method of reducing incidence, severity, or risk of disseminated intravascular coagulation in a patient having a respiratory viral infection, comprising administering to a patient in need thereof a compound that is a HIF prolyl hydroxylase inhibitor or a HIF-alpha stabilizer, or a pharmaceutically acceptable salt thereof.

163. A method of treating or preventing disseminated intravascular coagulation in a patient having a respiratory viral infection, comprising administering to a patient in need thereof a compound that is a HIF prolyl hydroxylase inhibitor or a HIF-alpha stabilizer, or a pharmaceutically acceptable salt thereof.

164. A method of reducing mortality and morbidity related to disseminated intravascular coagulation in a patient having a respiratory viral infection, comprising administering to a patient in need thereof a compound that is a HIF prolyl hydroxylase inhibitor or a HIF-alpha stabilizer, or a pharmaceutically acceptable salt thereof.

165. The method of any one of paragraphs 162-164, wherein the patient has a lower respiratory infection.

166. The method of any one of paragraphs 162-164, wherein the patient has an upper respiratory infection.

167. A method of reducing mortality and morbidity related to disseminated intravascular coagulation in a patient having a pulmonary viral infection, comprising administering to a patient in need thereof a compound that is a HIF prolyl hydroxylase inhibitor or a HIF-alpha stabilizer, or a pharmaceutically acceptable salt thereof.

168. A method of treating or preventing disseminated intravascular coagulation in a patient having a pulmonary viral infection, comprising administering to a patient in need thereof a compound that is a HIF prolyl hydroxylase inhibitor or a HIF-alpha stabilizer, or a pharmaceutically acceptable salt thereof.

169. A method of reducing incidence, severity, or risk of disseminated intravascular coagulation in a patient having a pulmonary viral infection, comprising administering to a patient in need thereof a compound that is a HIF prolyl hydroxylase inhibitor or a HIF-alpha stabilizer, or a pharmaceutically acceptable salt thereof.

170. The method of any one of paragraphs 162-169, wherein the viral infection is induced by or associated with a virus that could lead to viral pneumonia.

171. The method of any one of paragraphs 162-170, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

172. A method of treating or preventing disseminated intravascular coagulation in a patient infected with a coronavirus, comprising administering to a patient in need thereof a compound that is a HIF prolyl hydroxylase inhibitor or a HIF-alpha stabilizer, or a pharmaceutically acceptable salt thereof.

173. A method of reducing mortality and morbidity related to disseminated intravascular coagulation in a patient infected with a coronavirus, comprising administering to a patient in need thereof a compound that is a HIF prolyl hydroxylase inhibitor or a HIF-alpha stabilizer, or a pharmaceutically acceptable salt thereof.

174. A method of reducing incidence, severity, or risk of disseminated intravascular coagulation in a patient infected with a coronavirus, comprising administering to a patient in need thereof a compound that is a HIF prolyl hydroxylase inhibitor or a HIF-alpha stabilizer, or a pharmaceutically acceptable salt thereof.

175. The method of any one of paragraphs 162-174, wherein the patient is infected by a coronavirus that is SARS-CoV, MERS-CoV, or SARS-CoV-2.

176. The method of any one of paragraphs 162-175, wherein the patient has COVID-19.

177. The method of any one of paragraphs 162-176, wherein the disseminated intravascular coagulation is induced by or associated with the viral infection.

178. The method of paragraph 177, wherein the disseminated intravascular coagulation is induced by or associated with COVID-19.

179. The method of any one of paragraphs 162-178, wherein the patient is receiving mechanical ventilation.

180. A method of treating or preventing kidney diseases in a patient having a respiratory viral infection, comprising administering to a patient in need thereof a compound that is a HIF prolyl hydroxylase inhibitor or a HIF-alpha stabilizer, or a pharmaceutically acceptable salt thereof.

181. A method of reducing mortality and morbidity related to kidney diseases in a patient having a respiratory viral infection, comprising administering to a patient in need thereof a compound that is a HIF prolyl hydroxylase inhibitor or a HIF-alpha stabilizer, or a pharmaceutically acceptable salt thereof.

182. A method of reducing incidence, severity, or risk of kidney diseases in a patient having a respiratory viral infection, comprising administering to a patient in need thereof a compound that is a HIF prolyl hydroxylase inhibitor or a HIF-alpha stabilizer, or a pharmaceutically acceptable salt thereof.

183. The method of any one of paragraphs 180-182, wherein the patient has a lower respiratory infection.

184. The method of any one of paragraphs 180-103, wherein the patient has an upper respiratory infection.

185. A method of treating or preventing kidney diseases in a patient having a pulmonary viral infection, comprising administering to a patient in need thereof a compound that is a HIF prolyl hydroxylase inhibitor or a HIF-alpha stabilizer, or a pharmaceutically acceptable salt thereof.

186. A method of reducing mortality and morbidity related to kidney diseases in a patient having a pulmonary viral infection, comprising administering to a patient in need thereof a compound that is a HIF prolyl hydroxylase inhibitor or a HIF-alpha stabilizer, or a pharmaceutically acceptable salt thereof.

187. A method of reducing incidence, severity, or risk of kidney diseases in a patient having a pulmonary viral infection, comprising administering to a patient in need thereof a compound that is a HIF prolyl hydroxylase inhibitor or a HIF-alpha stabilizer, or a pharmaceutically acceptable salt thereof.

188. The method of any one of paragraphs 180-187, wherein the viral infection is induced by or associated with a virus that could lead to viral pneumonia.

189. The method of any one of paragraphs 180-188, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

190. A method of treating or preventing kidney diseases in a patient infected with a coronavirus, comprising administering to a patient in need thereof a compound that is a HIF prolyl hydroxylase inhibitor or a HIF-alpha stabilizer, or a pharmaceutically acceptable salt thereof.

191. A method of reducing mortality and morbidity related to kidney diseases in a patient infected with a coronavirus, comprising administering to a patient in need thereof a compound that is a HIF prolyl hydroxylase inhibitor or a HIF-alpha stabilizer, or a pharmaceutically acceptable salt thereof.

192. A method of reducing incidence, severity, or risk of kidney diseases in a patient infected with a coronavirus, comprising administering to a patient in need thereof a compound that is a HIF prolyl hydroxylase inhibitor or a HIF-alpha stabilizer, or a pharmaceutically acceptable salt thereof.

193. The method of any one of paragraphs 180-192, wherein the patient is infected by a coronavirus that is SARS-CoV, MERS-CoV, or SARS-CoV-2.

194. The method of any one of paragraphs 180-193, wherein the patient has COVID-19.

195. The method of any one of paragraphs 180-194, wherein the kidney diseases is induced by or associated with the viral infection.

196. The method of paragraph 195, wherein the kidney diseases is induced by or associated with COVID-19.

197. The method of any one of paragraphs 180-196, wherein the patient is receiving mechanical ventilation.

198. A method of treating or preventing liver failure in a patient having a respiratory viral infection, comprising administering to a patient in need thereof a compound that is a HIF prolyl hydroxylase inhibitor or a HIF-alpha stabilizer, or a pharmaceutically acceptable salt thereof.

199. A method of reducing mortality and morbidity related to liver failure in a patient having a respiratory viral infection, comprising administering to a patient in need thereof a compound that is a HIF prolyl hydroxylase inhibitor or a HIF-alpha stabilizer, or a pharmaceutically acceptable salt thereof.

200. A method of reducing incidence, severity, or risk of liver failure in a patient having a respiratory viral infection, comprising administering to a patient in need thereof a compound that is a HIF prolyl hydroxylase inhibitor or a HIF-alpha stabilizer, or a pharmaceutically acceptable salt thereof.

201. The method of any one of paragraphs 198-200, wherein the patient has a lower respiratory infection.

202. The method of any one of paragraphs 198-200, wherein the patient has an upper respiratory infection.

203. A method of treating or preventing liver failure in a patient having a pulmonary viral infection, comprising administering to a patient in need thereof a compound that is a HIF prolyl hydroxylase inhibitor or a HIF-alpha stabilizer, or a pharmaceutically acceptable salt thereof.

204. A method of reducing mortality and morbidity related to liver failure in a patient having a pulmonary viral infection, comprising administering to a patient in need thereof a compound that is a HIF prolyl hydroxylase inhibitor or a HIF-alpha stabilizer, or a pharmaceutically acceptable salt thereof.

205. A method of reducing incidence, severity, or risk of liver failure in a patient having a pulmonary viral infection, comprising administering to a patient in need thereof a compound that is a HIF prolyl hydroxylase inhibitor or a HIF-alpha stabilizer, or a pharmaceutically acceptable salt thereof.

206. The method of any one of paragraphs 198-205, wherein the viral infection is induced by or associated with a virus that could lead to viral pneumonia.

207. The method of any one of paragraphs 198-206, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

208. A method of treating or preventing liver failure in a patient infected with a coronavirus, comprising administering to a patient in need thereof a compound that is a HIF prolyl hydroxylase inhibitor or a HIF-alpha stabilizer, or a pharmaceutically acceptable salt thereof.

209. A method of reducing mortality and morbidity related to liver failure in a patient infected with a coronavirus, comprising administering to a patient in need thereof a compound that is a HIF prolyl hydroxylase inhibitor or a HIF-alpha stabilizer, or a pharmaceutically acceptable salt thereof.

210. A method of reducing incidence, severity, or risk of liver failure in a patient infected with a coronavirus, comprising administering to a patient in need thereof a compound that is a HIF prolyl hydroxylase inhibitor or a HIF-alpha stabilizer, or a pharmaceutically acceptable salt thereof.

211. The method of any one of paragraphs 198-210, wherein the patient is infected by a coronavirus that is SARS-CoV, MERS-CoV, or SARS-CoV-2.

212. The method of any one of paragraphs 198-211, wherein the patient has COVID-19.

213. The method of any one of paragraphs 198-212, wherein the liver failure is induced by or associated with the viral infection.

214. The method of paragraph 213, wherein the liver failure is induced by or associated with COVID-19.

215. The method of any one of paragraphs 198-214, wherein the patient is receiving mechanical ventilation.

216. A method of treating or preventing pancreas injury in a patient having a respiratory viral infection, comprising administering to a patient in need thereof a compound that is a HIF prolyl hydroxylase inhibitor or a HIF-alpha stabilizer, or a pharmaceutically acceptable salt thereof.

217. A method of reducing mortality and morbidity related to pancreas injury in a patient having a respiratory viral infection, comprising administering to a patient in need thereof a compound that is a HIF prolyl hydroxylase inhibitor or a HIF-alpha stabilizer, or a pharmaceutically acceptable salt thereof.

218. A method of reducing incidence, severity, or risk of pancreas injury in a patient having a respiratory viral infection, comprising administering to a patient in need thereof a compound that is a HIF prolyl hydroxylase inhibitor or a HIF-alpha stabilizer, or a pharmaceutically acceptable salt thereof.

219. The method of any one of paragraphs 216-218, wherein the patient has a lower respiratory infection.

220. The method of any one of paragraphs 216-218, wherein the patient has an upper respiratory infection.

221. A method of treating or preventing pancreas injury in a patient having a pulmonary viral infection, comprising administering to a patient in need thereof a compound that is a HIF prolyl hydroxylase inhibitor or a HIF-alpha stabilizer, or a pharmaceutically acceptable salt thereof.

222. A method of reducing mortality and morbidity related to pancreas injury in a patient having a pulmonary viral infection, comprising administering to a patient in need thereof a compound that is a HIF prolyl hydroxylase inhibitor or a HIF-alpha stabilizer, or a pharmaceutically acceptable salt thereof.

223. A method of reducing incidence, severity, or risk of pancreas injury in a patient having a pulmonary viral infection, comprising administering to a patient in need thereof a compound that is a HIF prolyl hydroxylase inhibitor or a HIF-alpha stabilizer, or a pharmaceutically acceptable salt thereof.

224. The method of any one of paragraphs 216-223, wherein the viral infection is induced by or associated with a virus that could lead to viral pneumonia.

225. The method of any one of paragraphs 216-224, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

226. A method of treating or preventing pancreas injury in a patient infected with a coronavirus, comprising administering to a patient in need thereof a compound that is a HIF prolyl hydroxylase inhibitor or a HIF-alpha stabilizer, or a pharmaceutically acceptable salt thereof.

227. A method of reducing mortality and morbidity related to pancreas injury in a patient infected with a coronavirus, comprising administering to a patient in need thereof a compound that is a HIF prolyl hydroxylase inhibitor or a HIF-alpha stabilizer, or a pharmaceutically acceptable salt thereof.

228. A method of reducing incidence, severity, or risk of pancreas injury in a patient infected with a coronavirus, comprising administering to a patient in need thereof a compound that is a HIF prolyl hydroxylase inhibitor or a HIF-alpha stabilizer, or a pharmaceutically acceptable salt thereof.

229. The method of any one of paragraphs 216-228, wherein the patient is infected by a coronavirus that is SARS-CoV, MERS-CoV, or SARS-CoV-2.

230. The method of any one of paragraphs 216-229, wherein the patient has COVID-19.

231. The method of any one of paragraphs 216-230, wherein pancreas injury is induced by or associated with the viral infection.

232. The method of paragraph 231, wherein the pancreas injury is induced by or associated with COVID-19.

233. The method of any one of paragraphs 216-232, wherein the patient is receiving mechanical ventilation.

234. A method of treating or preventing multi-organ failure in a patient having a respiratory viral infection, comprising administering to a patient in need thereof a compound that is a HIF prolyl hydroxylase inhibitor or a HIF-alpha stabilizer, or a pharmaceutically acceptable salt thereof.

235. A method of reducing mortality and morbidity related to multi-organ failure in a patient having a respiratory viral infection, comprising administering to a patient in need thereof a compound that is a HIF prolyl hydroxylase inhibitor or a HIF-alpha stabilizer, or a pharmaceutically acceptable salt thereof.

236. A method of reducing incidence, severity, or risk of multi-organ failure in a patient having a respiratory viral infection, comprising administering to a patient in need thereof a compound that is a HIF prolyl hydroxylase inhibitor or a HIF-alpha stabilizer, or a pharmaceutically acceptable salt thereof.

237. The method of any one of paragraphs 234-236, wherein the patient has a lower respiratory infection.

238. The method of any one of paragraphs 234-236, wherein the patient has an upper respiratory infection.

239. A method of treating or preventing multi-organ failure in a patient having a pulmonary viral infection, comprising administering to a patient in need thereof a compound that is a HIF prolyl hydroxylase inhibitor or a HIF-alpha stabilizer, or a pharmaceutically acceptable salt thereof.

240. A method of reducing mortality and morbidity related to multi-organ failure in a patient having a pulmonary viral infection, comprising administering to a patient in need thereof a compound that is a HIF prolyl hydroxylase inhibitor or a HIF-alpha stabilizer, or a pharmaceutically acceptable salt thereof.

241. A method of reducing incidence, severity, or risk of multi-organ failure in a patient having a pulmonary viral infection, comprising administering to a patient in need thereof a compound that is a HIF prolyl hydroxylase inhibitor or a HIF-alpha stabilizer, or a pharmaceutically acceptable salt thereof.

242. The method of any one of paragraphs 234-241, wherein the viral infection is induced by or associated with a virus that could lead to viral pneumonia.

243. The method of any one of paragraphs 234-242, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

244. A method of treating or preventing multi-organ failure in a patient having a coronavirus infection, comprising administering to a patient in need thereof a compound that is a HIF prolyl hydroxylase inhibitor or a HIF-alpha stabilizer, or a pharmaceutically acceptable salt thereof.

245. A method of reducing mortality and morbidity related to multi-organ failure in a patient infected with a coronavirus, comprising administering to a patient in need thereof a compound that is a HIF prolyl hydroxylase inhibitor or a HIF-alpha stabilizer, or a pharmaceutically acceptable salt thereof.

246. A method of reducing incidence, severity, or risk of multi-organ failure in a patient infected with a coronavirus, comprising administering to a patient in need thereof a compound that is a HIF prolyl hydroxylase inhibitor or a HIF-alpha stabilizer, or a pharmaceutically acceptable salt thereof.

247. The method of any one of paragraphs 234-246, wherein the patient is infected by a coronavirus that is SARS-CoV, MERS-CoV, or SARS-CoV-2.

248. The method of any one of paragraphs 234-247, wherein the patient has COVID-19.

249. The method of any one of paragraphs 234-248, wherein the multi-organ failure is induced by or associated with the viral infection.

250. The method of paragraph 249, wherein the multi-organ failure is induced by or associated with COVID-19.

251. The method of any one of paragraphs 234-249, wherein the patient is receiving mechanical ventilation.

252. A method of treating or preventing septic shock in a patient having a respiratory viral infection, comprising administering to a patient in need thereof a compound that is a HIF prolyl hydroxylase inhibitor or a HIF-alpha stabilizer, or a pharmaceutically acceptable salt thereof.

253. A method of reducing mortality and morbidity related to septic shock in a patient having a respiratory viral infection, comprising administering to a patient in need thereof a compound that is a HIF prolyl hydroxylase inhibitor or a HIF-alpha stabilizer, or a pharmaceutically acceptable salt thereof.

254. A method of reducing incidence, severity, or risk of septic shock in a patient having a respiratory viral infection, comprising administering to a patient in need thereof a compound that is a HIF prolyl hydroxylase inhibitor or a HIF-alpha stabilizer, or a pharmaceutically acceptable salt thereof.

255. The method of any one of paragraphs 252-254, wherein the patient has a lower respiratory infection.

256. The method of any one of paragraphs 252-254, wherein the patient has an upper respiratory infection.

257. A method of treating or preventing septic shock in a patient having a pulmonary viral infection, comprising administering to a patient in need thereof a compound that is a HIF prolyl hydroxylase inhibitor or a HIF-alpha stabilizer, or a pharmaceutically acceptable salt thereof.

258. A method of reducing mortality and morbidity related to septic shock in a patient having a pulmonary viral infection, comprising administering to a patient in need thereof a compound that is a HIF prolyl hydroxylase inhibitor or a HIF-alpha stabilizer, or a pharmaceutically acceptable salt thereof.

259. A method of reducing incidence, severity, or risk of septic shock in a patient having a pulmonary viral infection, comprising administering to a patient in need thereof a compound that is a HIF prolyl hydroxylase inhibitor or a HIF-alpha stabilizer, or a pharmaceutically acceptable salt thereof.

260. The method of any one of paragraphs 252-259, wherein the viral infection is induced by or associated with a virus that could lead to viral pneumonia.

261. The method of any one of paragraphs 252-260, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

262. A method of treating or preventing septic shock in a patient infected with a coronavirus, comprising administering to a patient in need thereof a compound that is a HIF prolyl hydroxylase inhibitor or a HIF-alpha stabilizer, or a pharmaceutically acceptable salt thereof.

263. A method of reducing mortality and morbidity related to septic shock in a patient infected with a coronavirus, comprising administering to a patient in need thereof a compound that is a HIF prolyl hydroxylase inhibitor or a HIF-alpha stabilizer, or a pharmaceutically acceptable salt thereof.

264. A method of reducing incidence, severity, or risk of septic shock in a patient infected with a coronavirus, comprising administering to a patient in need thereof a compound that is a HIF prolyl hydroxylase inhibitor or a HIF-alpha stabilizer, or a pharmaceutically acceptable salt thereof.

265. The method of any one of paragraphs 252-264, wherein the patient is infected by a coronavirus that is SARS-CoV, MERS-CoV, or SARS-CoV-2.

266. The method of any one of paragraphs 252-265, wherein the patient has COVID-19.

267. The method of any one of paragraphs 252-266, wherein the septic shock is induced by or associated with the viral infection.

268. The method of paragraph 267, wherein the septic shock is induced by or associated with COVID-19.

269. The method of any one of paragraphs 252-268, wherein the patient is receiving mechanical ventilation.

270. A method of treating or preventing sepsis in a patient having a respiratory viral infection, comprising administering to a patient in need thereof a compound that is a HIF prolyl hydroxylase inhibitor or a HIF-alpha stabilizer, or a pharmaceutically acceptable salt thereof.

271. A method of reducing mortality and morbidity related to sepsis in a patient having a respiratory viral infection, comprising administering to a patient in need thereof a compound that is a HIF prolyl hydroxylase inhibitor or a HIF-alpha stabilizer, or a pharmaceutically acceptable salt thereof.

272. A method of reducing incidence, severity, or risk of sepsis in a patient having a respiratory viral infection, comprising administering to a patient in need thereof a compound that is a HIF prolyl hydroxylase inhibitor or a HIF-alpha stabilizer, or a pharmaceutically acceptable salt thereof.

273. The method of any one of paragraphs 270-272, wherein the patient has a lower respiratory infection.

274. The method of any one of paragraphs 270-272, wherein the patient has an upper respiratory infection.

275. A method of treating or preventing sepsis in a patient having a pulmonary viral infection, comprising administering to a patient in need thereof a compound that is a HIF prolyl hydroxylase inhibitor or a HIF-alpha stabilizer, or a pharmaceutically acceptable salt thereof.

276. A method of reducing mortality and morbidity related to sepsis in a patient having a pulmonary viral infection, comprising administering to a patient in need thereof a compound that is a HIF prolyl hydroxylase inhibitor or a HIF-alpha stabilizer, or a pharmaceutically acceptable salt thereof.

277. A method of reducing incidence, severity, or risk of sepsis in a patient having a pulmonary viral infection, comprising administering to a patient in need thereof a compound that is a HIF prolyl hydroxylase inhibitor or a HIF-alpha stabilizer, or a pharmaceutically acceptable salt thereof.

278. The method of any one of paragraphs 270-277, wherein the viral infection is induced by or associated with a virus that could lead to viral pneumonia.

279. The method of any one of paragraphs 270-278, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

280. A method of treating or preventing sepsis in a patient infected with a coronavirus, comprising administering to a patient in need thereof a compound that is a HIF prolyl hydroxylase inhibitor or a HIF-alpha stabilizer, or a pharmaceutically acceptable salt thereof.

281. A method of reducing mortality and morbidity related to sepsis in a patient infected with a coronavirus, comprising administering to a patient in need thereof a compound that is a HIF prolyl hydroxylase inhibitor or a HIF-alpha stabilizer, or a pharmaceutically acceptable salt thereof.

282. A method of reducing incidence, severity, or risk of sepsis in a patient infected with a coronavirus, comprising administering to a patient in need thereof a compound that is a HIF prolyl hydroxylase inhibitor or a HIF-alpha stabilizer, or a pharmaceutically acceptable salt thereof.

283. The method of any one of paragraphs 270-282, wherein the patient is infected by a coronavirus that is SARS-CoV, MERS-CoV, or SARS-CoV-2.

284. The method of any one of paragraphs 270-283, wherein the patient has COVID-19.

285. The method of any one of paragraphs 270-284, wherein the sepsis is induced by or associated with the viral infection.

286. The method of paragraph 285, wherein the sepsis is induced by or associated with COVID-19.

287. The method of any one of paragraph 270-286, wherein the patient is receiving mechanical ventilation.

288. A method of treating or preventing cytokine release syndrome in a patient having a respiratory viral infection, comprising administering to a patient in need thereof a compound that is a HIF prolyl hydroxylase inhibitor or a HIF-alpha stabilizer, or a pharmaceutically acceptable salt thereof.

289. A method of reducing mortality and morbidity related to cytokine release syndrome in a patient having a respiratory viral infection, comprising administering to a patient in need thereof a compound that is a HIF prolyl hydroxylase inhibitor or a HIF-alpha stabilizer, or a pharmaceutically acceptable salt thereof.

290. A method of reducing incidence, severity, or risk of cytokine release syndrome in a patient having a respiratory viral infection, comprising administering to a patient in need thereof a compound that is a HIF prolyl hydroxylase inhibitor or a HIF-alpha stabilizer, or a pharmaceutically acceptable salt thereof.

291. The method of any one of paragraphs 288-291, wherein the patient has a lower respiratory infection.

292. The method of any one of paragraphs 288-291, wherein the patient has an upper respiratory infection.

293. A method of treating or preventing cytokine release syndrome in a patient having a pulmonary viral infection, comprising administering to a patient in need thereof a compound that is a HIF prolyl hydroxylase inhibitor or a HIF-alpha stabilizer, or a pharmaceutically acceptable salt thereof.

294. A method of reducing mortality and morbidity related to cytokine release syndrome in a patient having a pulmonary viral infection, comprising administering to a patient in need thereof a compound that is a HIF prolyl hydroxylase inhibitor or a HIF-alpha stabilizer, or a pharmaceutically acceptable salt thereof.

295. A method of reducing incidence, severity, or risk of cytokine release syndrome in a patient having a pulmonary viral infection, comprising administering to a patient in need thereof a compound that is a HIF prolyl hydroxylase inhibitor or a HIF-alpha stabilizer, or a pharmaceutically acceptable salt thereof.

296. The method of any one of paragraphs 288-295, wherein the viral infection is induced by or associated with a virus that could lead to viral pneumonia.

297. The method of any one of paragraphs 288-296, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

298. A method of treating or preventing cytokine release syndrome in a patient infected with a coronavirus, comprising administering to a patient in need thereof a compound that is a HIF prolyl hydroxylase inhibitor or a HIF-alpha stabilizer, or a pharmaceutically acceptable salt thereof.

299. A method of reducing mortality and morbidity related to cytokine release syndrome in a patient infected with a coronavirus, comprising administering to a patient in need thereof a compound that is a HIF prolyl hydroxylase inhibitor or a HIF-alpha stabilizer, or a pharmaceutically acceptable salt thereof.

300. A method of reducing incidence, severity, or risk of cytokine release syndrome in a patient infected with a coronavirus, comprising administering to a patient in need thereof a compound that is a HIF prolyl hydroxylase inhibitor or a HIF-alpha stabilizer, or a pharmaceutically acceptable salt thereof.

301. The method of any one of paragraphs 288-300, wherein the patient is infected by a coronavirus that is SARS-CoV, MERS-CoV, or SARS-CoV-2.

302. The method of any one of paragraphs 288-301, wherein the patient has COVID-19.

303. The method of any one of paragraphs 288-302, wherein the cytokine release syndrome is induced by or associated with the viral infection.

304. The method of paragraph 303, wherein the cytokine release syndrome is induced by or associated with COVID-19.

305. The method of any one of paragraphs 288-304, wherein the patient is receiving mechanical ventilation.

306. A method of treating or preventing neurological disorder in a patient having a respiratory viral infection, comprising administering to a patient in need thereof a compound that is a HIF prolyl hydroxylase inhibitor or a HIF-alpha stabilizer, or a pharmaceutically acceptable salt thereof.

307. A method of reducing mortality and morbidity related to neurological disorder in a patient having a respiratory viral infection, comprising administering to a patient in need thereof a compound that is a HIF prolyl hydroxylase inhibitor or a HIF-alpha stabilizer, or a pharmaceutically acceptable salt thereof.

308. A method of reducing incidence, severity, or risk of neurological disorder in a patient having a respiratory viral infection, comprising administering to a patient in need thereof a compound that is a HIF prolyl hydroxylase inhibitor or a HIF-alpha stabilizer, or a pharmaceutically acceptable salt thereof.

309. The method of any one of paragraphs 306-308, wherein the patient has a lower respiratory infection.

310. The method of any one of paragraphs 306-308, wherein the patient has an upper respiratory infection.

311. A method of treating or preventing neurological disorder in a patient having a pulmonary viral infection, comprising administering to a patient in need thereof a compound that is a HIF prolyl hydroxylase inhibitor or a HIF-alpha stabilizer, or a pharmaceutically acceptable salt thereof.

312. A method of reducing mortality and morbidity related to neurological disorder in a patient having a pulmonary viral infection, comprising administering to a patient in need thereof a compound that is a HIF prolyl hydroxylase inhibitor or a HIF-alpha stabilizer, or a pharmaceutically acceptable salt thereof.

313. A method of reducing incidence, severity, or risk of neurological disorder in a patient having a pulmonary viral infection, comprising administering to a patient in need thereof a compound that is a HIF prolyl hydroxylase inhibitor or a HIF-alpha stabilizer, or a pharmaceutically acceptable salt thereof.

314. The method of any one of paragraphs 306-313, wherein the viral infection is induced by or associated with a virus that could lead to viral pneumonia.

315. The method of any one of paragraphs 306-314, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

316. A method of treating or preventing neurological disorder in a patient infected with a coronavirus, comprising administering to a patient in need thereof a compound that is a HIF prolyl hydroxylase inhibitor or a HIF-alpha stabilizer, or a pharmaceutically acceptable salt thereof.

317. A method of reducing mortality and morbidity related to neurological disorder in a patient infected with a coronavirus, comprising administering to a patient in need thereof a compound that is a HIF prolyl hydroxylase inhibitor or a HIF-alpha stabilizer, or a pharmaceutically acceptable salt thereof.

318. A method of reducing incidence, severity, or risk of neurological disorder in a patient infected with a coronavirus, comprising administering to a patient in need thereof a compound that is a HIF prolyl hydroxylase inhibitor or a HIF-alpha stabilizer, or a pharmaceutically acceptable salt thereof.

319. The method of any one of paragraphs 306-318, wherein the patient is infected by a coronavirus that is SARS-CoV, MERS-CoV, or SARS-CoV-2.

320. The method of any one of paragraphs 306-319, wherein the patient has COVID-19.

321. The method of any one of paragraphs 306-320, wherein the neurological disorder is induced by or associated with the viral infection.

322. The method of paragraph 321, wherein the neurological disorder is induced by or associated with COVID-19.

323. The method of any one of paragraphs 306-322, wherein the patient is receiving mechanical ventilation.

324. A method of treating or preventing pulmonary barotrauma in a patient having a respiratory viral infection, comprising administering to a patient in need thereof a compound that is a HIF prolyl hydroxylase inhibitor or a HIF-alpha stabilizer, or a pharmaceutically acceptable salt thereof.

325. A method of reducing mortality and morbidity related to pulmonary barotrauma in a patient having a respiratory viral infection, comprising administering to a patient in need thereof a compound that is a HIF prolyl hydroxylase inhibitor or a HIF-alpha stabilizer, or a pharmaceutically acceptable salt thereof.

326. A method of reducing incidence, severity, or risk pulmonary barotrauma in a patient having a respiratory viral infection, comprising administering to a patient in need thereof a compound that is a HIF prolyl hydroxylase inhibitor or a HIF-alpha stabilizer, or a pharmaceutically acceptable salt thereof.

327. The method of any one of paragraphs 324-326, wherein the patient has a lower respiratory infection.

328. The method of any one of paragraphs 324-326, wherein the patient has an upper respiratory infection.

329. A method of treating or preventing pulmonary barotrauma in a patient having a pulmonary viral infection, comprising administering to a patient in need thereof a compound that is a HIF prolyl hydroxylase inhibitor or a HIF-alpha stabilizer, or a pharmaceutically acceptable salt thereof.

330. A method of reducing mortality and morbidity related to pulmonary barotrauma in a patient having a pulmonary viral infection, comprising administering to a patient in need thereof a compound that is a HIF prolyl hydroxylase inhibitor or a HIF-alpha stabilizer, or a pharmaceutically acceptable salt thereof.

331. A method of reducing incidence, severity, or risk of pulmonary barotrauma in a patient having a pulmonary viral infection, comprising administering to a patient in need thereof a compound that is a HIF prolyl hydroxylase inhibitor or a HIF-alpha stabilizer, or a pharmaceutically acceptable salt thereof.

332. The method of any one of paragraphs 324-331, wherein the viral infection is induced by or associated with a virus that could lead to viral pneumonia.

333. The method of any one of paragraphs 324-332, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

334. A method of treating or preventing pulmonary barotrauma in a patient infected with a coronavirus, comprising administering to a patient in need thereof a compound that is a HIF prolyl hydroxylase inhibitor or a HIF-alpha stabilizer, or a pharmaceutically acceptable salt thereof.

335. A method of reducing mortality and morbidity related to pulmonary barotrauma in a patient infected with a coronavirus, comprising administering to a patient in need thereof a compound that is a HIF prolyl hydroxylase inhibitor or a HIF-alpha stabilizer, or a pharmaceutically acceptable salt thereof.

336. A method of reducing incidence, severity, or risk of pulmonary barotrauma in a patient infected with a coronavirus, comprising administering to a patient in need thereof a compound that is a HIF prolyl hydroxylase inhibitor or a HIF-alpha stabilizer, or a pharmaceutically acceptable salt thereof.

337. The method of any one of paragraphs 324-336, wherein the patient is infected by a coronavirus that is SARS-CoV, MERS-CoV, or SARS-CoV-2.

338. The method of any one of paragraphs 324-337, wherein the patient has COVID-19.

339. The method of any one of paragraphs 324-338, wherein the pulmonary barotrauma is induced by or associated with the viral infection.

340. The method of paragraph 339, wherein the pulmonary barotrauma is induced by or associated with COVID-19.

341. The method of any one of paragraphs 324-340, wherein the patient is receiving mechanical ventilation.

342. A method of treating or preventing organ dysfunction induced by or associated with acute lung injury (ALI), acute respiratory distress syndrome (ARDS), pneumonia, lung inflammation, or any combination thereof, comprising administering to a patient in need thereof a compound that is a HIF prolyl hydroxylase inhibitor or a HIF-alpha stabilizer, or a pharmaceutically acceptable salt thereof.

343. The method of paragraph 342, wherein the patient is infected with coronavirus.

344. The method of paragraph 342 or 343, wherein the patient is infected by a coronavirus that is SARS-CoV, MERS-CoV, or SARS-CoV-2.

345. The method of any one of paragraphs 342-344, wherein the patient has COVID-19.

346. The method of any one of paragraphs 342-345, wherein the patient has a respiratory viral infection.

347. The method of paragraph 346, wherein the patient has a lower respiratory infection.

348. The method of paragraph 346, wherein the patient has an upper respiratory infection.

349. The method of paragraph 342, wherein the patient has a pulmonary viral infection.

350. A method for improving lung volumes in a patient in need thereof, comprising administering to a patient in need thereof a compound that is a HIF prolyl hydroxylase inhibitor or a HIF-alpha stabilizer, or a pharmaceutically acceptable salt thereof.

351. A method for improving pulmonary compliance in a patient in need thereof, comprising administering to a patient in need thereof a compound that is a HIF prolyl hydroxylase inhibitor or a HIF-alpha stabilizer, or a pharmaceutically acceptable salt thereof.

352. A method for promoting fibroproliferative repair in a patient in need thereof, comprising administering to a patient in need thereof a compound that is a HIF prolyl hydroxylase inhibitor or a HIF-alpha stabilizer, or a pharmaceutically acceptable salt thereof.

353. A method for regulating innate and adaptive immunity, comprising administering to a patient in need thereof a compound that is a HIF prolyl hydroxylase inhibitor or a HIF-alpha stabilizer, or a pharmaceutically acceptable salt thereof.

354. A method for promoting regulatory T-cells, lymphocytes and B cells, comprising administering to a patient in need thereof a compound that is a HIF prolyl hydroxylase inhibitor or a HIF-alpha stabilizer, or a pharmaceutically acceptable salt thereof.

355. A method for treating/preventing bronchial inflammation, comprising administering to a patient in need thereof a compound that is a HIF prolyl hydroxylase inhibitor or a HIF-alpha stabilizer, or a pharmaceutically acceptable salt thereof.

356. A method for reducing cytokine production, comprising administering to a patient in need thereof a compound that is a HIF prolyl hydroxylase inhibitor or a HIF-alpha stabilizer, or a pharmaceutically acceptable salt thereof.

357. A method for improving dyspnea, comprising administering to a patient in need thereof a compound that is a HIF prolyl hydroxylase inhibitor or a HIF-alpha stabilizer, or a pharmaceutically acceptable salt thereof.

358. A method for improving arterial PaO₂, comprising administering to a patient in need thereof a compound that is a HIF prolyl hydroxylase inhibitor or a HIF-alpha stabilizer, or a pharmaceutically acceptable salt thereof.

359. The method of any one of paragraphs 350-358, wherein the patient has a viral infection.

360. The method of paragraph 359, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

361. The method of paragraph 360, wherein the viral infection is induced by or associated with coronavirus.

362. The method of paragraph 361, wherein the coronavirus is the SARS-CoV, MERS-CoV, or SARS-CoV-2.

363. The method of any one of paragraphs 350-362, wherein the patient has COVID-19.

364. A method of treating or preventing acute respiratory distress syndrome (ARDS) in a hospitalized patient with COVID-19, the method comprising administering to said patient a compound that is a HIF prolyl hydroxylase inhibitor or a HIF-alpha stabilizer, or a pharmaceutically acceptable salt thereof.

365. The method of any one of paragraphs 101-364, wherein said patient is an adult.

366. The method of any one of paragraphs 101-365, wherein said patient exhibits one or more symptoms of hypoxia.

367. The method of paragraph 366, wherein the patient has a lower oxygen saturation of hemoglobin compared to normal oxygen saturation.

368. The method of any one of paragraphs 101-367, wherein the patient has an oxygen saturation of hemoglobin that is about 94% or less.

369. The method of any one of paragraphs 1-368, wherein the patient has a pre-existing respiratory or pulmonary disease.

370. The method of paragraph 369, wherein the pre-existing respiratory or pulmonary disease is acute lung injury, bronchitis, pneumonia, pulmonary fibrosis, asthma, and acute respiratory distress syndrome, or pulmonary hypertension.

371. The method of any one of paragraphs 1-368, wherein the patient does not have a pre-existing respiratory or pulmonary disease.

372. The method of any one of paragraphs 1-371, wherein the patient is an adult aged 45 years and above, aged 65 years and above, aged 80 years and above, over 80 years of age, aged 45-60 years, aged 60-80 years, or has underlying medical conditions.

373. The method of paragraph 372, wherein the underlying medical conditions comprise liver disease, heart conditions, kidney disease, obesity, diabetes, or being immunocompromised.

374. The method of paragraph 373, wherein the patient has a body mass index (BMI) that is 25 or above or is 30 or above.

375. The method of paragraphs 1-374, wherein the patient has one or more clinically-recognized symptoms associated with the respiratory viral infection or the coronavirus infection, wherein the respiratory viral infection or the coronavirus infection is COVID-19.

376. The method of paragraph 375, wherein the one or more symptoms associated with COVID-19 are fever, dry cough, fatigue, coughing up sputum from the lungs, bone or joint pain, sore throat, headache, chills, nausea or vomiting, stuffy nose, pressure or pain in the chest, shortness of breath, sudden confusion, digestive issues, conjunctivitis, bluish face or lips, or loss of smell or taste.

377. The method of any one of paragraphs 1-376, wherein the patient has decreased oxygen saturation relative to normal levels.

378. The method of paragraph 377, wherein the decreased oxygen saturation is an oxygen saturation of about 94% or less.

379. The method of any one of paragraphs 1-376, wherein the patient has an elevated level of an inflammation marker.

380. The method of paragraph 379, wherein the elevated level of an inflammation marker is detected in the serum of the patient.

381. The method of paragraph 380, wherein the elevated level of an inflammation marker is detected in the lung of the patient.

382. The method of paragraph 381, wherein the inflammation marker is a presence of ground-glass opacity.

383. The method of paragraphs 1-382, wherein the patient has a positive diagnostic test for SARS-CoV-2.

384. The method of any one of paragraphs 1-383, wherein the compound stabilizes HIFα.

385. The method of any one of paragraphs 1-384, wherein the compound inhibits hydroxylation of HIFα.

386. The method of any one of paragraphs 1-385, wherein the HIF prolyl hydroxylase inhibitor or the HIF-alpha stabilizer has a structure of Formula (I), Formula (II) or Formula (III)

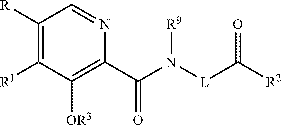

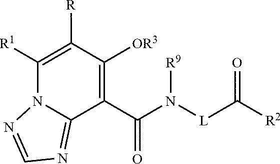

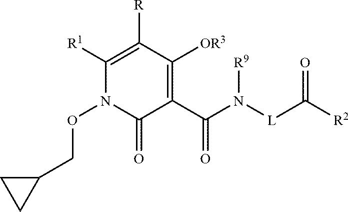

or a pharmaceutically acceptable salt thereof, wherein R and R¹ are each independently:
(i) hydrogen
(ii) substituted or unsubstituted phenyl;
(iii) substituted or unsubstituted heteroaryl; or
(iv) substituted or unsubstituted alkyl;
said substitution selected from:
   (i) C₁-C₄ alkyl;
   (ii) C₃-C₄ cycloalkyl;
   (iii) C₁-C₄ alkoxy;
   (iV) C₃-C₄ cycloalkoxy;
   (v) C₁-C₄ haloalkyl;
   (vi) C₃-C4 halocycloalkyl;
   (vii) halogen;
   (viii) cyano;
   (ix) NHC(O)R⁴;
   (x) C(O)NR^{5a}R^{5b}_{;}
   (xi) phenyl; and
   (xii) heteroaryl; or
   (xiii) two substituents are taken together to form a fused ring having from 5 to 7 atoms;
R⁴ is a C₁-C₄ alkyl or C₃-C₄ cycloalkyl;
R^{5a} and R^{5b} are each independently selected from:
   (i) hydrogen;
   (ii) C₁-C₄ alkyl;
   (iii) C₃-C₄ cycloalkyl; or
   (iv) R^{5a} and R^{5b} are taken together to form a ring having from 3 to 7 atoms;
R² is selected from:
   (i) OR⁶
   (ii) NR^{7a}R^{7b}; and
R⁶ is selected from hydrogen and C₁-C₄ alkyl or C₃-C₄ cycloalkyl; R^{7a} and R^{7b} are each independently selected from:
   (i) hydrogen;
   (ii) C₁-C₄ alkyl or C₃-C₄ cycloalkyl; or
   (iii) R^{7a} and R^{7b} are taken together to form a ring having from 3 to 7 atoms;
R³ is selected from hydrogen, methyl, and ethyl;
L is a linking unit having a structure -[C(R^{8a}R^{8b})]ₙ-R^{8a} and R^{8b} are each independently selected from hydrogen, methyl and ethyl;
n is an integer from 1 to 3; and
R⁹ is selected from hydrogen and methyl.

387. The method of any one of paragraphs 1-385, wherein the HIF prolyl hydroxylase inhibitor or the HIF-alpha stabilizer has a structure of Formula (IV)

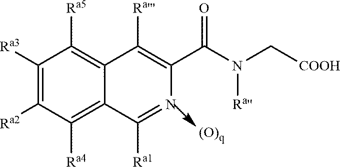

or a pharmaceutically acceptable salt thereof, wherein
q is zero or one;
R^{a1} is selected from the group consisting of hydrogen, alkyl, Substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted hetero cyclic, and R^{a7} is hydrogen, alkyl or aryl or, when X is —NR^{a7}— , then R^{a7} and R^{a6}, together with the nitrogen atom to which they are bound, can be joined to form a heterocyclic or substituted heterocyclic group;
R^{a2} and R^{a3} are independently selected from the group consisting of hydrogen, alkyl, substituted alkyl, aryl, substituted aryl, heteroaryl, substituted heteroaryl, halo, hydroxy, cyano. —S(O)ₙ—N(R^{a6})—R^{a6} where n is 0, 1, or 2, —NR^{a6}C(O)NR^{a6}R^{a6}, —XR^{a6} where X is oxygen, —S(O)ₙ—, or —NR^{a7}— where n is zero, one or two, each R^{a6} is independently selected from the group consisting of hydrogen, alkyl, Substituted alkyl, aryl, substituted aryl, cycloalkyl, substituted cycloalkyl, heteroaryl, substituted heteroaryl, heterocyclic and substituted heterocyclic provided that when when X is —SO— or —SO₂— , then R^{a6} is not hydrogen, and R^{a7} is selected from the group consisting of hydrogen, alkyl, aryl, or R^{a2}, R^{a3} together with the carbon atom pendent thereto, form an aryl substituted aryl, heteroaryl, or substituted heteroaryl;
R^{a4} and R^{a5} are independently selected from the group consisting of hydrogen, halo, alkyl, substituted alkyl, alkoxy, Substituted alkoxy, aryl, substituted aryl, heteroaryl, substituted heteroaryl and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl or, when X is —NR^{a7}—, then R^{a7} and R^{a6}, together with the nitrogen atom to which they are bound, can be joined to form a heterocyclic or Substituted heterocyclic group;
R^{a″} is selected from the group consisting of hydrogen, alkyl and substituted alkyl;
R^{a‴} is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)—R^{a10} wherein R^{a10} is selected from the group consisting of alkyl, substituted alkyl, cycloalkyl, substituted cycloalkyl, aryl, substituted aryl, heteroaryl and substituted heteroaryl and n is zero, one or two;
or a pharmaceutically acceptable salt, ester, or prodrug thereof;
with the proviso that when R^{a″} is hydrogen and q is zero, then at least one of the following occurs:
1) R^{a1} is fluoro, bromo, iodo, alkyl, substituted alkyl, alkoxy, aminoacyl, Substituted alkoxy, aryl, substituted aryl, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl; or
2) R^{a2} is substituted alkyl, aryl, substituted aryl, heteroaryl, substituted heteroaryl, fluoro, bromo, iodo, cyano, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a2} is substituted alkyl such a substituent does not include trifluoromethyl;
   b) —XR^{a6} is not alkoxy; and
   c) when —XR^{a6} is substituted alkoxy such a substituent does not include benzyl or benzyl substituted by a substituent selected from the group consisting of (C₁-C₅) alkyl and (C₁-C₅) alkoxy or does not include a fluoroalkoxy substituent of the formula:
   —O—[CH₂]ₓ—C_{f}H_{(2f+1-g)}F_{g}
where x is zero or one; f is an integer of from 1 to 5; and
g is an integer of from 1 to (2f+1); or
3) R^{a3} is substituted alkyl, aryl, substituted aryl, heteroaryl, substituted heteroaryl, bromo, iodo, —XR^{a6} where X is oxygen—S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a3} is substituted alkyl such a substituent does not include trifluoromethyl;
   b) —XR^{a6} is not alkoxy; and
   c) when XR^{a6} is substituted alkoxy such a substituent does not include benzyl or benzyl substituted by a substituent selected from the group consisting of (C₁-C₅) alkyl and (C-C) alkoxy or does not include a fluoroalkoxy substituent of the formula:
   —O—[CH2]_{x—}C_{f}H_{(2f+1-g)}F_{g}
where x is zero or one; f is an integer of from 1 to 5; and
g is an integer of from 1 to (2f+1); or
4) R^{a4} is iodo, substituted alkyl, aryl, substituted aryl, heteroaryl, substituted heteroaryl, —XR^{a6} where X is oxygen—S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a4} is substituted alkyl such a substituent does not include trifluoromethyl;
   b) —XR^{a6} is not alkoxy; and
   c) when XR^{a6} is substituted alkoxy such a substituent does not include benzyl or benzyl substituted by a substituent selected from the group consisting of (C₁-C₅) alkyl and (C-C) alkoxy or does not include a fluoroalkoxy substituent of the formula:
   —O—[CH₂]ₓ—C_{f}H_{(2f+1-g)}F_{g}
where x is zero or one; f is an integer of from 1 to 5; and
g is an integer of from 1 to (2f+1); or
5) R^{a5} is iodo, substituted alkyl, aryl, substituted aryl, heteroaryl, substituted heteroaryl, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R²⁵ is substituted alkyl such a substituent does not include trifluoromethyl;
   b) —XR^{a6} is not alkoxy; and
   c) when XR^{a6} is substituted alkoxy such a substituent does not include benzyl or benzyl substituted by a substituent selected from the group consisting of (C₁-C₅) alkyl and (C-C) alkoxy or does not include a fluoroalkoxy substituent of the formula:
   —O—[CH₂]ₓ—C_{f}H_{(2f+1-g)}F_{g}
where x is zero or one; f is an integer of from 1 to 5; and
g is an integer of from 1 to (2f+1);
and with the further following proviso:
that when R^{a1}, R^{a3}, R^{a4}, and R^{a5} are hydrogen, then R^{a2} is not bromo.

388. The method of any one of paragraphs 1-385, wherein the HIF prolyl hydroxylase inhibitor or the HIF-alpha stabilizer has a structure of Formula (V)

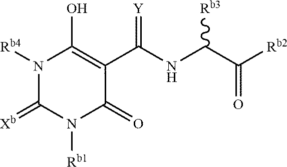

or a pharmaceutically acceptable salt thereof, wherein
R^{b1} and R^{b4} are each independently selected from the group consisting of hydrogen, — NR^{b5}R^{b6}, C₁-C₁₀ alkyl, C₂-C₁₀ alkenyl, C₂-C₁₀ alkynyl, C₃-C₈ cycloalkyl, C₃-C₈ cycloalkyl-C₁-C₁₀ alkyl, C₅-C₈ cycloalkenyl, C₅-C₈ cycloalkenyl- C₁-C₁₀ alkyl, C₃-C₈ heterocycloalkyl, C₃-C₈ heterocycloalkyl—C₁—Cₗo alkyl, aryl, aryl- C₁-C₁₀ alkyl, heteroaryl and heteroaryl- C₁-C₁₀ alkyl;
R^{b2} is —NR^{b7}R^{b8} or —OR^{b9};
R^{b3} is H or C₁-C₄ alkyl;
where R^{b5} and R^{b6} are each independently selected from the group consisting of hydrogen, C₁-C₁₀ alkyl, C₃-C₈ cycloalkyl, C₃-C₈ cycloalkyl-C₁-C₁₀ alkyl, C₃-C₈ heterocycloalkyl, C₃-C₈ heterocycloalkyl—C₁—C₁₀ alkyl, aryl, aryl- C₁-C₁₀ alkyl, heteroaryl and heteroaryl- C₁-C₁₀ alkyl, —C(O)C₁—C₄ alkyl, —C(O)C₃—C₆ cycloalkyl, —C(O)C₃—C₆ heterocycloalkyl, —C(O)aryl, —C(O)heteroaryl and —S(O)₂ C₁—C₄ alkyl, or, when R^{b5} and R^{b6} are attached to the same nitrogen, R^{b5} and R^{b6} taken together with the nitrogen to which they are attached forma 5- or 6- or 7-membered saturated ring optionally containing one other heteroatom selected from oxygen, nitrogen and sulphur,
R^{b7} and R^{b8} are each independently selected from the group consisting of hydrogen, C₁-C₁₀ alkyl, C₂-C₁₀ alkenyl, C₃-C₈ cycloalkyl, C₃-C₈ heterocycloalkyl, aryl, and heteroaryl, and
R^{b9} is H or a cation, or C₁-C₁₀ alkyl which is unsubstituted or substituted with one or more substituents independently selected from the group consisting of C₃-C₆ cycloalkyl, heterocycloalkyl, aryl and heteroaryl;
X^{b} is O or S; and
Y is O or S;
where any carbon or heteroatom of R^{b1}, R^{b2}, R^{b3}, R^{b4}, R^{b5}, R^{b6}, R^{b7}, R^{b8}, R^{b9} is unsubstituted or is substituted with one or more substituents independently selected from C₁-C₆ alkyl, C₁-C₆ haloalkyl, halogen, —OR^{b10}, —NR^{b5}R^{b6}, oxo, cyano, nitro, —C(O)R^{b10}, —C(O)OR^{b10}, —SR^{b10}, —S(O)R^{b10}, —S(O)₂R^{b10}, —CONR^{b5}R^{b6}, —N(R^{b5})C(O)R^{b10}, —N(R^{b5})C(O)OR^{b10},
—OC(O)NR^{b5}R^{b6}′, —N(R^{b5})C(O)NR^{b5}R^{b6}, —SO₂NR^{b5}R^{b6}, —N(R^{b5})SO₂R^{b10}, C₂-C₁₀ alkenyl,
C₂-C₁₀ alkynyl, C₃-C₆ cycloalkyl, C₃-C₆ heterocycloalkyl, aryl, C₁-C₆ alkyl-aryl, heteroaryl and C₁-C₆ alkyl-heteroaryl, wherein R^{b5} and R^{b6} are the same as defined above and R^{b10} is selected from hydrogen, C₁-C₁₀ alkyl, C₂-C₁₀ alkenyl, C₂-C₁₀ alkynyl. — C(O)C₁—C₄ alkyl, —C(O)aryl,
—C(O)heteroaryl, —C(O)C₃—C₆ cycloalkyl, —C(O)C₃—C₆ heterocycloalkyl, —S(O)₂C₁—C₄ alkyl, C₃-C₈ cycloalkyl, C₃-C₈ heterocycloalkyl, C₆-C₁₄ aryl, aryl-C₁-C₁₀ alkyl, heteroaryl and heteroaryl-C₁-C₁₀ alkyl;

389. The method of any one of paragraphs 1-385, wherein the HIF prolyl hydroxylase inhibitor or the HIF-alpha stabilizer has a structure of Formula (VI)

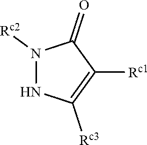

or a pharmaceutically acceptable salt thereof, wherein
R^{c1} represents a heteroaryl group of the formula

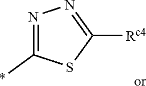

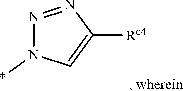

* denotes the linkage point with the dihydropyrazolone ring and
R^{c4} denotes hydrogen, fluorine, chlorine, bromine, cyano, C₁-C₄ alkyl, trifluoromethyl, hydroxymethyl, C₁-C₄ alkoxy, trifluoromethoxy, hydroxycarbonyl or C₁-C₄ alkoxycarbonyl;
R^{c2} represents a heteroaryl group of the formula

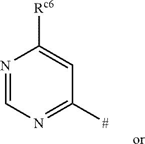

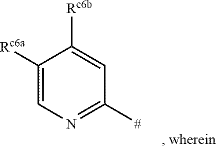

# denotes the linkage point with the dihydropyrazolone ring and
R^{c6}, R^{c6a} and R^{c6b} are identical or different and independently of one another denote hydrogen or a substituent chosen from the group consisting of fluorine, chlorine, bromine, cyano, C₁-C₆ alkyl, trifluoromethyl, hydroxyl, C₁-C₆ alkoxy, trifluoromethoxy, amino, mono-C₁-C₄ alkylamino, di-C₁-C₄ alkylamino, hydroxycarbonyl, C₁-C₄ alkoxycarbonyl, 4-to 6-membered heterocycloalkyl, phenyl and 5-or 6-membered heteroaryl, wherein
C₁-C₆ alkyl in its turn can be substituted by hydroxyl, C₁-C₄ alkoxy or amino and 4-to 6-membered heterocycloalkyl, phenyl and 5-or 6-membered heteroaryl in their turn can in each case be substituted once or twice in an identical or different manner by fluorine, chlorine, bromine, cyano, C₁-C₄ alkyl, trifluoromethyl, hydroxyl, C₁-C₄ alkoxy, trifluoromethoxy, oxo, amino, mono-C₁-C₄ alkylamino, di-C₁-C₄ alkylamino, hydroxycarbonyl or C₁-C₄ alkoxycarbonyl,
and R^{c3} represents hydrogen, or a salt thereof.

390. The method of any one of paragraphs 1-389, wherein the HIF prolyl hydroxylase inhibitor or a HIF-alpha stabilizer, or a pharmaceutically acceptable salt thereof, is administered orally, intravenously, intramuscularly, by inhalation or transdermally.

391. The method of paragraph 390, wherein the HIF prolyl hydroxylase inhibitor or a HIF-alpha stabilizer, or a pharmaceutically acceptable salt thereof, is administered orally.

392. The method of any one of paragraphs 1-391, wherein the HIF prolyl hydroxylase inhibitor or a HIF-alpha stabilizer, or a pharmaceutically acceptable salt thereof, is administered to a subject patient at a dose of about 100-1500, 100-1800, or 150-1800 mg per day.

393. The method of any one of paragraphs 1-389, wherein the HIF prolyl hydroxylase inhibitor or the HIF-alpha stabilizer is vadadustat (AKB-6548), roxadustat (FG-4592), daprodustat (GSK-12788363), molidustat (BAY 85-3934), enarodustat (JTZ-951) or desidustat (ZYAN1).

394. The method of paragraph 393, wherein the HIF prolyl hydroxylase inhibitor or the HIF-alpha stabilizer is vadadustat (AKB-6548).

395. The method of paragraph 394, wherein vadadustat (AKB-6548) is administered at a dose of 150 mg to 1000 mg or of 150 mg to 1800 mg per day.

396. The method of paragraph 395, wherein vadadustat (AKB-6548) is administered at an initial dose of 900 mg per day.

397. The method of paragraph 394 or 395, wherein vadadustat (AKB-6548) is administered at a dose of 900 mg per day throughout the treatment period.

398. The method of paragraph 394 or 395, comprising administration of vadadustat (AKB-6548) a dose of 900-1800 mg per day.

399. The method of paragraph 398, comprising administration of vadadustat (AKB-6548) at a dose of 900, 1050, 1200, 1350, 1500, 1650, or 1800 mg per day.

400. The method of any one of paragraphs 1-399, wherein the HIF prolyl hydroxylase inhibitor or the HIF-alpha stabilizer is administered to the patient for up to about one month.

401. The method of paragraph 400, wherein the HIF prolyl hydroxylase inhibitor or the HIF-alpha stabilizer is administered to the patient for up to about four weeks (about 28 days), three weeks (about 21 days), two weeks (about 14 days), or one week (about 7 days).

402. The method of any one of paragraphs 1-401, wherein the HIF prolyl hydroxylase inhibitor or the HIF-alpha stabilizer is administered in combination with another treatment.

403. The method of paragraph 402, wherein the treatment comprises an antiviral agent or oxygen therapy.

404. The method of paragraph 403, wherein oxygen therapy comprises mechanical ventilation.

405. The method of any one of paragraphs 1-404, wherein the severity of disease is maintained or reduced following commencement of administering the HIF prolyl hydroxylase inhibitor or the HIF-alpha stabilizer to the patient.

406. The method of aragraph 405, wherein the severity of disease is assessed according to the following ordinal scale where increasing numbers denote increased severity:

| Score | Descriptor | Patient State | |
|---|---|---|---|
| 0 | No clinical or virological evidence of infection | Uninfected | Uninfected/ Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | | |
| Score | Descriptor | Patient State | |
| 6 | Patient is hospitalized and receives oxygen therapy that is non-invasive ventilation or high flow oxygen devices | Infected -Hospitalized (Severe Disease) | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |

407. The method of paragraph 405 or 406, wherein the severity of disease is maintained and the patient’s condition stabilizes following commencement of administering the HIF prolyl hydroxylase inhibitor or the HIF-alpha stabilizer to the patient.

408. The method of paragraph 406, wherein the patient’s score does not change and the patient’s condition stabilizes following commencement of administering the HIF prolyl hydroxylase inhibitor or the HIF-alpha stabilizer to the patient.

409. The method of paragraph 405 or 406, wherein the severity of disease decreases following commencement of administering the HIF prolyl hydroxylase inhibitor or the HIF-alpha stabilizer to the patient.

410. The method of paragraph 406, wherein the patient’s score decreases by at least one point following commencement of administering the HIF prolyl hydroxylase inhibitor or the HIF-alpha stabilizer to the patient.

411. The method of paragraph 406, wherein the patient’s severity of disease is assessed as uninfected/ambulatory.

412. The method of paragraph 406, wherein the patient’s severity of disease is assessed as hospitalized but not intubated.

413. The method of paragraph 406, wherein the patient’s severity of disease is assessed as hospitalized and intubated.

414. The method of paragraph 406, wherein the patient’s category decreases by at least one category following commencement of administering the HIF prolyl hydroxylase inhibitor or the HIF-alpha stabilizer to the patient

415. The method of any one of paragraphs 405-410, wherein the patient is hypoxic.

416. The method of paragraph 415, wherein the patient has an oxygen saturation value that is about 94% or less.

417. The method of aragraph 415 or 416, wherein the patient’s oxygen saturation value improves to a value of about 95% or greater following commencement of administering the HIF prolyl hydroxylase inhibitor or the HIF-alpha stabilizer to the patient.

418. The method of any one of paragraphs 405-417, wherein the patient has hypotension.

419. The method of paragraph 418, wherein the patient receives vasopressor therapy.

420. The method of paragraph 419, wherein vasopressor therapy is reduced or discontinued following commencement of administering the HIF prolyl hydroxylase inhibitor or the HIF-alpha stabilizer to the patient.

421. The method of any one of paragraphs 405-420, wherein the patient is hospitalized.

422. The method of paragraph 421, wherein the patient does not receive oxygen therapy.

423. The method of paragraph 421, wherein the patient has a score of 1, 2, and/or 3 according to the ordinal scale of paragraph 406.

424. The method of paragraph 421, wherein the patient receives oxygen therapy.

425. The method of paragraph 424, wherein the patient has a score of 4, 5, 6, or 7 according to the ordinal scale of paragraph 406.

426. The method of paragraph 424 or 425, wherein the patient receives oxygen therapy by mask or nasal prongs.

427. The method of paragraph 424 or 425, wherein the patient receives oxygen therapy by intubation and mechanical ventilation.

428. The method of paragraph 427, wherein the patient has multi-organ injuries and receives additional organ support therapy.

429. The method of paragraph 427, wherein the patient has any of a cardiovascular injury, a neurological injury, a liver injury, a pancreas injury, and/or a kidney disease.

430. The method of paragraph 429, wherein the patient has a heart injury and/or a lung injury.

431. The method of any one of paragraphs 427-430, wherein the patient receives organ support therapy that is vapopressor therapy, renal replacement therapy (RRT), and/or extracorporeal membrane oxygenation (ECMO).

432. The method of any one of aragraphs 427-431, wherein the organ support therapy received by the patient is discontinued following commencement of administering the HIF prolyl hydroxylase inhibitor or the HIF-alpha stabilizer to the patient.

433. The method of any one of paragraphs 424-432, wherein the oxygen therapy received by the patient is discontinued following commencement of administering the HIF prolyl hydroxylase inhibitor or the HIF-alpha stabilizer to the patient.

434. The method of any one of paragraphs 427-433, wherein the patient has increased ventilator-free survival following discontinuation of mechanical ventilation.

435. The method of any one of paragraphs 405-434, wherein the patient receives vadadustat, or a pharmaceutically acceptable salt thereof.

### SECOND SET OF CERTAIN EXEMPLARY EMBODIMENTS

Still further exemplary embodiments are described in the following numbered paragraphs 1-197 of this second set of exemplary embodiments.

1. A method of treating a respiratory viral infection, comprising administering to a patient in need thereof a compound that is a HIF prolyl hydroxylase inhibitor or a HIF-alpha stabilizer, or a pharmaceutically acceptable salt thereof.

2. The method of paragraph 1, wherein the respiratory viral infection is induced by or associated with a virus that could lead to viral pneumonia.

3. The method of paragraph 1 or 2, wherein the respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus.

4. The method of any one of paragraphs 1-3, wherein the respiratory viral infection is induced by or associated with a coronavirus.

5. The method of paragraph 4, wherein the coronavirus is the SARS-CoV, MERS-CoV, or SARS-CoV-2.

6. The method of any one of paragraphs 1-5, wherein the respiratory viral infection is lower respiratory infection.

7. The method of any one of paragraphs 1-6, wherein the respiratory viral infection is pulmonary viral infection.

8. The method of any one of paragraphs 1-5, wherein the respiratory viral infection is upper respiratory infection.

9. The method of any one of paragraphs 1-8, wherein the respiratory viral infection is COVID-19.

10. The method of any one of paragraphs 1-9, wherein the respiratory viral infection induces a lung disease.

11. The method of paragraph 10, wherein the lung disease is selected from acute lung injury (ALI), bronchitis, pneumonia, pulmonary fibrosis, asthma, or acute respiratory distress syndrome (ARDS).

12. The method of paragraph 10 or 11, wherein the patient develops pulmonary hypertension.

13. The method of paragraph 10 or 11, wherein the patient develops multi-organ failure.

14. The method of paragraph 13, wherein the multi-organ failure comprise heart failure, liver failure, lung failure kidney failure, and gastrointestinal (GI) system failure.

15. A method of treating a coronavirus infection, comprising administering to a patient in need thereof a compound that is a HIF prolyl hydroxylase inhibitor or a HIF-alpha stabilizer, or a pharmaceutically acceptable salt thereof.

16. A method of treating or preventing a lung disease that is acute lung injury (ALI) in a patient in need thereof, comprising administering to the patient a compound that is a HIF prolyl hydroxylase inhibitor or a HIF-alpha stabilizer, or a pharmaceutically acceptable salt thereof, optionally wherein said ALI is induced by or associated with a ventilator.

17. The method of paragraph 16, wherein said patient has a viral infection, sepsis, pneumonia, aspiration, trauma, pancreatitis, blood transfusion, and/or smoke or toxic gas inhalation.

18. The method of paragraph 16 or 17, wherein said acute lung injury (ALI) is induced by or associated with a viral infection, sepsis, pneumonia, aspiration, trauma, pancreatitis, blood transfusion, and/or smoke or toxic gas inhalation.

19. The method of any one of paragraphs 16-18, wherein said patient is mechanically ventilated.

20. A method of treating or preventing a lung disease that is acute lung injury (ALI) in a patient having a respiratory viral infection, comprising administering to the patient a compound that is a HIF prolyl hydroxylase inhibitor or a HIF-alpha stabilizer, or a pharmaceutically acceptable salt thereof.

21. A method of treating or preventing a lung disease that is acute respiratory distress syndrome (ARDS) in a patient having a respiratory viral infection, comprising administering to the patient a compound that is a HIF prolyl hydroxylase inhibitor or a HIF-alpha stabilizer, or a pharmaceutically acceptable salt thereof.

22. The method of paragraph 20 or 21, wherein the respiratory viral infection is a pulmonary infection.

23. The method of any one of paragraphs 20-22, wherein the respiratory viral infection is infection by a coronavirus, an influenza A, an influenza B, a Rhinovirus, a respiratory syncytial virus (RSV), an adenovirus, a human parainfluenza virus (HPIV) or an enterovirus.

24. The method of any one of paragraphs 20-23, wherein the respiratory viral infection is infection by a coronavirus.

25. The method of paragraph 24, wherein the coronavirus is the SARS-CoV, MERS-CoV, or SARS-CoV-2.

26. The method of any one of paragraphs 20-25, wherein the respiratory viral infection is COVID-19.

27. The method of paragraph 20, wherein the patient with acute lung injury develops acute respiratory distress syndrome (ARDS).

28. The method of any one of paragraphs 20-27, wherein the patient with ALI or ARDS develops pulmonary hypertension.

29. The method of any one of paragraphs 20-27, wherein the patient with ALI or ARDS develops multi-organ failure.

30. The method of paragraph 29, wherein the multi-organ failure comprise heart failure, liver failure, lung failure kidney failure, and gastrointestinal (GI) system failure.

31. The method of any one of paragraphs 20-30, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

32. The method of any one of paragraphs 20-31, wherein ALI or ARDS is induced by or associated with the viral infection.

33. A method of treating or preventing a lung disease that is acute lung injury (ALI) in a patient infected with a coronavirus, comprising administering to the patient a compound that is a HIF prolyl hydroxylase inhibitor or a HIF-alpha stabilizer, or a pharmaceutically acceptable salt thereof.

34. A method of treating or preventing a lung disease that is acute respiratory distress syndrome (ARDS) in a patient infected with a coronavirus, comprising administering to the patient a compound that is a HIF prolyl hydroxylase inhibitor or a HIF-alpha stabilizer, or a pharmaceutically acceptable salt thereof.

35. The method of paragraph 33 or 34, wherein the coronavirus is the SARS-CoV, MERS-CoV, or SARS-CoV-2.

36. The method of any one of paragraphs 33-35, wherein the patient is infected with SARS-CoV-2.

37. The method of any one of paragraphs 33-36, wherein the patient with ALI or ARDS develops pulmonary hypertension and/or multi-organ failure, optionally wherein the multi-organ failure comprise heart failure, liver failure, lung failure kidney failure, and gastrointestinal (GI) system failure.

38. The method of any one of paragraphs 33-37, wherein ALI or ARDS is characterized by accumulation of inflammatory cells into the lungs, cytokine release, inflammatory activation of recruited or resident cells, disruption of the alveolar-capillary barrier function, pulmonary edema, attenuated gas exchange, or lung inflammation, or any combination thereof.

39. The method of any one of paragraphs 33-38, wherein ALI or ARDS is induced by or associated with the viral infection.

40. A method of reducing mortality and morbidity related to acute lung injury (ALI) or acute respiratory distress syndrome (ARDS) or reducing incidence, severity, or risk of acute lung injury (ALI) or acute respiratory distress syndrome (ARDS) in a patient having a respiratory viral infection, said method comprising administering to a patient in need thereof a compound that is a HIF prolyl hydroxylase inhibitor or a HIF-alpha stabilizer, or a pharmaceutically acceptable salt thereof, optionally wherein the respiratory viral infection is a pulmonary viral infection.

41. The method of paragraph 40, wherein the patient has a lower respiratory infection.

42. The method of paragraph 40, wherein the patient has an upper respiratory infection.

43. The method of any one of paragraphs 40-42, wherein the viral infection is induced by or associated with a virus that could lead to viral pneumonia.

44. The method of any one of paragraphs 40-43, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

45. A method of reducing mortality and morbidity related to acute lung injury (ALI) or acute respiratory distress syndrome (ARDS) in a patient infected with a coronavirus or reducing incidence, severity, or risk of acute lung injury (ALI) or acute respiratory distress syndrome (ARDS), said method comprising administering to a patient in need thereof a compound that is a HIF prolyl hydroxylase inhibitor or a HIF-alpha stabilizer, or a pharmaceutically acceptable salt thereof.

46. The method of any one of paragraphs 40-45, wherein the acute lung injury (ALI) or acute respiratory distress syndrome (ARDS) is induced by or associated with the virus.

47. The method of any one of paragraphs 40-46, wherein the patient is infected by a coronavirus that is SARS-CoV, MERS-CoV, or SARS-CoV-2.

48. The method of any one of paragraphs 40-47, wherein the patient has COVID-19.

49. The method of any one of paragraphs 40-48, wherein the patient is receiving mechanical ventilation.

50. The method of any one of paragraphs 40-49, wherein the patient has ventilator induced ALI.

51. A method of treating or preventing lung inflammation or pneumonia, reducing mortality and morbidity related to lung inflammation or pneumonia, or reducing incidence, severity, or risk of lung inflammation or pneumonia in a patient having a respiratory viral infection, said method comprising administering to a patient in need thereof a compound that is a HIF prolyl hydroxylase inhibitor or a HIF-alpha stabilizer, or a pharmaceutically acceptable salt thereof, and optionally wherein the respiratory viral infection is a pulmonary viral infection.

52. The method of paragraph 51, wherein the patient has a lower respiratory infection.

53. The method of paragraph 51, wherein the patient has an upper respiratory infection.

54. The method of any one of aragraphs 51-53, wherein the viral infection is induced by or associated with a virus that could lead to viral pneumonia.

55. The method of any one of paragraphs 51-54, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

56. A method of treating or preventing lung inflammation or pneumonia, reducing mortality and morbidity related to lung inflammation or pneumonia, or reducing incidence, severity, or risk of lung inflammation or pneumonia in a patient infected with a coronavirus, said method comprising administering to a patient in need thereof a compound that is a HIF prolyl hydroxylase inhibitor or a HIF-alpha stabilizer, or a pharmaceutically acceptable salt thereof.

57. The method of any one of paragraphs 51-56, wherein the patient is infected by a coronavirus that is SARS-CoV, MERS-CoV, or SARS-CoV-2.

58. The method of any one of paragraphs 51-57, wherein the patient has COVID-19.

59. The method of any one of paragraphs 51-58, wherein the lung inflammation or pneumonia is induced by or associated with the viral infection, optionally wherein the lung inflammation or pneumonia is induced by or associated with COVID-19.

60. A method of treating or preventing COVID-19 related pneumonia, comprising administering to a patient in need thereof a compound that is a HIF prolyl hydroxylase inhibitor or a HIF-alpha stabilizer, or a pharmaceutically acceptable salt thereof, and

wherein the patient has acute lung injury(ALI), acute respiratory distress syndrome (ARDS), and/or organ failure, optionally wherein any of the acute lung injury (ALI), acute respiratory distress syndrome (ARDS), and/or organ failure is COVID-19 related.

61. A method of treating or preventing COVID-19 related acute lung injury (ALI) or COVID-19 related acute respiratory distress syndrome (ARDS) in a patient having pneumonia, comprising administering to a patient in need thereof a compound that is a HIF prolyl hydroxylase inhibitor or a HIF-alpha stabilizer, or a pharmaceutically acceptable salt thereof.

62. A method of treating or preventing COVID-19 related organ failure, comprising administering to a patient in need thereof a compound that is a HIF prolyl hydroxylase inhibitor or a HIF-alpha stabilizer, or a pharmaceutically acceptable salt thereof.

63. The method of any one of paragraphs 51-62, wherein the patient is receiving mechanical ventilation.

64. A method of treating or preventing cardiac dysfunction, reducing mortality and morbidity related to an adverse cardiac event, or reducing incidence, severity, or risk of an adverse cardiac event in a patient having a respiratory viral infection, said method comprising administering to a patient in need thereof a compound that is a HIF prolyl hydroxylase inhibitor or a HIF-alpha stabilizer, or a pharmaceutically acceptable salt thereof, optionally wherein the respiratory viral infection is a pulmonary viral infection.

65. A method of treating or preventing hypotension in a patient having a respiratory viral infection, reducing mortality and morbidity related to hypotension, or reducing incidence, severity, or risk of hypotension
said method comprising administering to a patient in need thereof a compound that is a HIF prolyl hydroxylase inhibitor or a HIF-alpha stabilizer, or a pharmaceutically acceptable salt thereof,
optionally wherein the respiratory viral infection is a pulmonary viral infection.

66. A method of reducing incidence, severity, or risk of disseminated intravascular coagulation, treating or preventing disseminated intravascular coagulation, or reducing mortality and morbidity related to disseminated intravascular coagulation in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein the respiratory viral infection is a pulmonary viral infection.

67. A method of treating or preventing kidney diseases, reducing mortality and morbidity related to kidney diseases, or reducing incidence, severity, or risk of kidney diseases in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein the respiratory viral infection is a pulmonary viral infection.

68. A method of treating or preventing liver failure, reducing mortality and morbidity related to liver failure, or reducing incidence, severity, or risk of liver failure in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein said respiratory viral infection is a pulmonary viral infection.

69. A method of treating or preventing pancreas injury, reducing mortality and morbidity related to pancreas injury, or reducing incidence, severity, or risk of pancreas injury in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein said respiratory viral infection is a pulmonary viral infection.

70. A method of treating or preventing multi-organ failure, reducing mortality and morbidity related to multi-organ failure, or reducing incidence, severity, or risk of multi-organ failure in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein the respiratory viral infection is a pulmonary viral infection.

71. A method of treating or preventing septic shock, reducing mortality and morbidity related to septic shock, or reducing incidence, severity, or risk of septic shock in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein the respiratory viral infection is a pulmonary viral infection.

72. A method of treating or preventing sepsis, reducing mortality and morbidity related to sepsis, or reducing incidence, severity, or risk of sepsis in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein said respiratory viral infection is a pulmonary viral infection.

73. A method of treating or preventing cytokine release syndrome, reducing mortality and morbidity related to cytokine release syndrome, or reducing incidence, severity, or risk of cytokine release syndrome in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein the respiratory viral infection is a pulmonary viral infection.

74. A method of treating or preventing neurological disorder, reducing mortality and morbidity related to neurological disorder, or reducing incidence, severity, or risk of neurological disorder in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein said respiratory viral infection is a pulmonary viral infection.

75. A method of treating or preventing pulmonary barotrauma, reducing mortality and morbidity related to pulmonary barotrauma, or reducing incidence, severity, or risk related to pulmonary barotrauma in a patient having a respiratory viral infection,
said method comprising administering to a patient in need thereof a compound that is a HIF prolyl hydroxylase inhibitor or a HIF-alpha stabilizer, or a pharmaceutically acceptable salt thereof,
optionally wherein said respiratory viral infection is a pulmonary viral infection.

76. The method of any one of paragraphs 64-75, wherein the patient has a lower respiratory infection.

77. The method of any one of paragraphs 64-75, wherein the patient has an upper respiratory infection.

78. The method of any one of paragraphs 64-77, wherein the viral infection is induced by or associated with a virus that could lead to viral pneumonia.

79. The method of any one of paragraphs 64-78, wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

80. A method of treating or preventing cardiac dysfunction, reducing mortality and morbidity related to an adverse cardiac event, or reducing incidence, severity, or risk of an adverse cardiac event in a patient infected with a coronavirus, said method comprising administering to a patient in need thereof a compound that is a HIF prolyl hydroxylase inhibitor or a HIF-alpha stabilizer, or a pharmaceutically acceptable salt thereof.

81. A method of treating or preventing hypotension, reducing mortality and morbidity related to hypotension, or reducing incidence, severity, or risk of hypotension in a patient infected with a coronavirus, said method comprising administering to a patient in need thereof a compound that is a HIF prolyl hydroxylase inhibitor or a HIF-alpha stabilizer, or a pharmaceutically acceptable salt thereof.

82. A method of treating or preventing disseminated intravascular coagulation, reducing mortality and morbidity related to disseminated intravascular coagulation, or reducing incidence, severity, or risk of disseminated intravascular coagulation in a patient infected with a coronavirus, said method comprising administering to a patient in need thereof a compound that is a HIF prolyl hydroxylase inhibitor or a HIF-alpha stabilizer, or a pharmaceutically acceptable salt thereof.

83. A method of treating or preventing kidney diseases, reducing mortality and morbidity related to kidney diseases, or reducing incidence, severity, or risk of kidney diseases in a patient infected with a coronavirus, said method comprising administering to a patient in need thereof a compound that is a HIF prolyl hydroxylase inhibitor or a HIF-alpha stabilizer, or a pharmaceutically acceptable salt thereof.

84. A method of treating or preventing liver failure, reducing mortality and morbidity related to liver failure, or reducing incidence, severity, or risk of liver failure in a patient infected with a coronavirus, said method comprising administering to a patient in need thereof a compound that is a HIF prolyl hydroxylase inhibitor or a HIF-alpha stabilizer, or a pharmaceutically acceptable salt thereof.

85. A method of treating or preventing pancreas injury, reducing mortality and morbidity related to pancreas injury, or reducing incidence, severity, or risk of pancreas injury in a patient infected with a coronavirus, said method comprising administering to a patient in need thereof a compound that is a HIF prolyl hydroxylase inhibitor or a HIF-alpha stabilizer, or a pharmaceutically acceptable salt thereof.

86. A method of treating or preventing multi-organ failure, reducing mortality and morbidity related to multi-organ failure, or reducing incidence, severity, or risk of multi-organ failure in a patient having a coronavirus infection, said method comprising administering to a patient in need thereof a compound that is a HIF prolyl hydroxylase inhibitor or a HIF-alpha stabilizer, or a pharmaceutically acceptable salt thereof.

87. A method of treating or preventing septic shock, reducing mortality and morbidity related to septic shock, or reducing incidence, severity, or risk of septic shock in a patient infected with a coronavirus, said method comprising administering to a patient in need thereof a compound that is a HIF prolyl hydroxylase inhibitor or a HIF-alpha stabilizer, or a pharmaceutically acceptable salt thereof.

88. A method of treating or preventing sepsis, reducing mortality and morbidity related to sepsis, or reducing incidence, severity, or risk of sepsis in a patient infected with a coronavirus, said method comprising administering to a patient in need thereof a compound that is a HIF prolyl hydroxylase inhibitor or a HIF-alpha stabilizer, or a pharmaceutically acceptable salt thereof.

89. A method of treating or preventing cytokine release syndrome, reducing mortality and morbidity related to cytokine release syndrome, or reducing incidence, severity, or risk of cytokine release syndrome in a patient infected with a coronavirus, said method comprising administering to a patient in need thereof a compound that is a HIF prolyl hydroxylase inhibitor or a HIF-alpha stabilizer, or a pharmaceutically acceptable salt thereof.

90. A method of treating or preventing neurological disorder, reducing mortality and morbidity related to neurological disorder, or reducing incidence, severity, or risk of neurological disorder in a patient infected with a coronavirus, said method comprising administering to a patient in need thereof a compound that is a HIF prolyl hydroxylase inhibitor or a HIF-alpha stabilizer, or a pharmaceutically acceptable salt thereof.

91. A method of treating or preventing pulmonary barotrauma, reducing mortality and morbidity related to pulmonary barotrauma, or reducing incidence, severity, or risk of pulmonary barotrauma in a patient infected with a coronavirus, said method comprising administering to a patient in need thereof a compound that is a HIF prolyl hydroxylase inhibitor or a HIF-alpha stabilizer, or a pharmaceutically acceptable salt thereof.

92. The method of any one of paragraphs 64-91, wherein the patient is infected by a coronavirus that is SARS-CoV, MERS-CoV, or SARS-CoV-2.

93. The method of any one of paragraphs 64-92, wherein the patient has COVID-19.

94. The method of paragraph 64 or 80, wherein the cardiac dysfunction is induced by or associated with the viral infection, optionally wherein the cardiac dysfunction is induced by or associated with COVID-19.

95. The method of paragraph 64 or 80 wherein an adverse cardiac event is induced by or associated with the viral infection, optionally wherein an adverse cardiac event is induced by or associated with COVID-19.

96. The method of paragraph 65 or 81, wherein the hypotension is induced by or associated with the viral infection, optionally wherein the hypotension is induced by or associated with COVID-19.

97. The method of paragraph 66 or 82, wherein the disseminated intravascular coagulation is induced by or associated with the viral infection, optionally wherein the disseminated intravascular coagulation is induced by or associated with COVID-19.

98. The method of paragraph 67 or 83, wherein the kidney diseases is induced by or associated with the viral infection, optionally wherein the kidney diseases is induced by or associated with COVID-19.

99. The method of any paragraph 68 or 84, wherein the liver failure is induced by or associated with the viral infection, optionally wherein the liver failure is induced by or associated with COVID-19.

100. The method of paragraph 69 or 85, wherein the pancreas injury is induced by or associated with the viral infection, optionally wherein the pancreas injury is induced by or associated with COVID-19.

101. The method of paragraph 70 or 86, wherein the multi-organ failure is induced by or associated with the viral infection, optionally wherein the multi-organ failure is induced by or associated with COVID-19.

102. The method of paragraph 71 or 87, wherein the septic shock is induced by or associated with the viral infection, optionally wherein the septic shock is induced by or associated with COVID-19.

103. The method of paragraph 72 or 88, wherein the sepsis is induced by or associated with the viral infection, optionally wherein the sepsis is induced by or associated with COVID-19.

104. The method of paragraph 73 or 89, wherein the cytokine release syndrome is induced by or associated with the viral infection, optionally wherein the cytokine release syndrome is induced by or associated with COVID-19.

105. The method of paragraph 74 or 90, wherein the neurological disorder is induced by or associated with the viral infection, optionally wherein the neurological disorder is induced by or associated with COVID-19.

106. The method of paragraph 75 or 91, wherein the pulmonary barotrauma is induced by or associated with the viral infection, optionally wherein the pulmonary barotrauma is induced by or associated with COVID-19.

107. The method of any one of paragraphs 64-106, wherein the patient is receiving mechanical ventilation.

108. A method of treating or preventing organ dysfunction induced by or associated with acute lung injury (ALI), acute respiratory distress syndrome (ARDS), pneumonia, lung inflammation, or any combination thereof, comprising administering to a patient in need thereof a compound that is a HIF prolyl hydroxylase inhibitor or a HIF-alpha stabilizer, or a pharmaceutically acceptable salt thereof.

109. The method of paragraph 108, wherein the patient is infected with coronavirus.

110. The method of paragraph 108 or 109, wherein the patient is infected by a coronavirus that is SARS-CoV, MERS-CoV, or SARS-CoV-2.

111. The method of any one of paragraphs 108-110, wherein the patient has COVID-19.

112. The method of any one of paragraphs 108-111, wherein the patient has a respiratory viral infection, optionally wherein the respiratory viral infection is a pulmonary infection.

113. The method of paragraph 112, wherein the patient has a lower respiratory infection.

114. The method of paragraph 112, wherein the patient has an upper respiratory infection.

115. A method for improving lung volumes in a patient in need thereof, comprising administering to a patient in need thereof a compound that is a HIF prolyl hydroxylase inhibitor or a HIF-alpha stabilizer, or a pharmaceutically acceptable salt thereof.

116. A method for improving pulmonary compliance in a patient in need thereof, comprising administering to a patient in need thereof a compound that is a HIF prolyl hydroxylase inhibitor or a HIF-alpha stabilizer, or a pharmaceutically acceptable salt thereof.

117. A method for promoting fibroproliferative repair in a patient in need thereof, comprising administering to a patient in need thereof a compound that is a HIF prolyl hydroxylase inhibitor or a HIF-alpha stabilizer, or a pharmaceutically acceptable salt thereof.

118. A method for regulating innate and adaptive immunity, comprising administering to a patient in need thereof a compound that is a HIF prolyl hydroxylase inhibitor or a HIF-alpha stabilizer, or a pharmaceutically acceptable salt thereof.

119. A method for promoting regulatory T-cells, lymphocytes and B cells, comprising administering to a patient in need thereof a compound that is a HIF prolyl hydroxylase inhibitor or a HIF-alpha stabilizer, or a pharmaceutically acceptable salt thereof.

120. A method for treating/preventing bronchial inflammation, comprising administering to a patient in need thereof a compound that is a HIF prolyl hydroxylase inhibitor or a HIF-alpha stabilizer, or a pharmaceutically acceptable salt thereof.

121. A method for reducing cytokine production, comprising administering to a patient in need thereof a compound that is a HIF prolyl hydroxylase inhibitor or a HIF-alpha stabilizer, or a pharmaceutically acceptable salt thereof.

122. A method for improving dyspnea, comprising administering to a patient in need thereof a compound that is a HIF prolyl hydroxylase inhibitor or a HIF-alpha stabilizer, or a pharmaceutically acceptable salt thereof.

123. A method for improving arterial PaO2, comprising administering to a patient in need thereof a compound that is a HIF prolyl hydroxylase inhibitor or a HIF-alpha stabilizer, or a pharmaceutically acceptable salt thereof.

124. The method of any one of paragraphs 115-123, wherein the patient has a viral infection, optionally wherein the viral infection is induced by or associated with a coronavirus, an influenza A, an influenza B, a Rhinovirus, a respiratory syncytial virus (RSV), an adenovirus, a human parainfluenza virus (HPIV) or an enterovirus.

125. The method of paragraph 124, wherein the viral infection is induced by or associated with coronavirus, optionally wherein the coronavirus is the SARS-CoV, MERS-CoV, or SARS-CoV-2.

126. The method of any one of paragraphs 115-125, wherein the patient has COVID-19.

127. The method of any one of paragraphs 1-126, wherein said patient is an adult.

128. The method of any one of paragraphs 1-127, wherein said patient exhibits one or more symptoms of hypoxia, optionally wherein the one or more symptoms of hypoxia include changes in the color of the skin, confusion, cough, fast heart rate, rapid breathing, shortness of breath, sweating, and/or wheezing; and/or said patient has a low oxygen saturation of hemoglobin, optionally an oxygen saturation of hemoglobin that is about 94% or less.

129. A method of treating or preventing acute respiratory distress syndrome (ARDS) in a hospitalized patient with COVID-19, the method comprising administering to said patient a compound that is a HIF prolyl hydroxylase inhibitor or a HIF-alpha stabilizer, or a pharmaceutically acceptable salt thereof.

130. The method of paragraph 129, wherein said patient is an adult.

131. The method of paragraph 129 or 130, wherein said patient exhibits one or more symptoms of hypoxia.

132. The method of paragraph 131, wherein the patient has a lower oxygen saturation of hemoglobin compared to normal oxygen saturation.

133. The method of any one of paragraphs 129-132, wherein the patient has an oxygen saturation of hemoglobin that is about 94% or less.

134. The method of any one of paragraphs 1-133, wherein the patient has a pre-existing respiratory or pulmonary disease.

135. The method of paragraph 134, wherein the pre-existing respiratory or pulmonary disease is acute lung injury, bronchitis, pneumonia, pulmonary fibrosis, asthma, and acute respiratory distress syndrome, or pulmonary hypertension.

136. The method of any one of paragraphs 1-133, wherein the patient does not have a pre-existing respiratory or pulmonary disease.

137. The method of any one of paragraphs 1-136, wherein the patient is an adult aged 45 years and above, aged 65 years and above, aged 80 years and above, over 80 years of age, aged 45-60 years, aged 60-80 years, or has underlying medical conditions.

138. The method of paragraph 137, wherein the underlying medical conditions comprise liver disease, heart conditions, kidney disease, obesity, diabetes, or being immunocompromised, optionally wherein the patient has a body mass index (BMI) that is 25 or above or is 30 or above.

139. The method of any one of paragraphs 1-138, wherein the patient has one or more clinically-recognized symptoms associated with the respiratory viral infection or the coronavirus infection, wherein the respiratory viral infection or the coronavirus infection is COVID-19, optionally wherein the one or more symptoms associated with COVID-19 are fever, dry cough, fatigue, coughing up sputum from the lungs, bone or joint pain, sore throat, headache, chills, nausea or vomiting, stuffy nose, pressure or pain in the chest, shortness of breath, sudden confusion, digestive issues, conjunctivitis, bluish face or lips, or loss of smell or taste.

140. The method of any one of paragraphs 1-139, wherein the patient has decreased oxygen saturation relative to normal levels, optionally wherein the decreased oxygen saturation is an oxygen saturation of about 94% or less.

141. The method of any one of paragraphs 1-140, wherein the patient has an elevated level of an inflammation marker.

142. The method of paragraph 141, wherein the elevated level of an inflammation marker is detected in the serum of the patient.

143. The method of paragraph 142, wherein the elevated level of an inflammation marker is detected in the lung of the patient.

144. The method of paragraph 143, wherein the inflammation marker is a presence of ground-glass opacity.

145. The method of paragraphs 1-144, wherein the patient has a positive diagnostic test for SARS-CoV-2.

146. The method of any one of paragraphs 1-145, wherein the compound stabilizes HIFα.

147. The method of any one of paragraphs 1-146, wherein the compound inhibits hydroxylation of HIFα.

148. The method of any one of paragraphs 1-147, wherein the HIF prolyl hydroxylase inhibitor or the HIF-alpha stabilizer has a structure of Formula (I), Formula (II) or Formula (III)

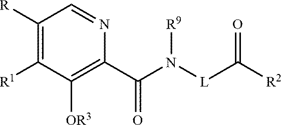

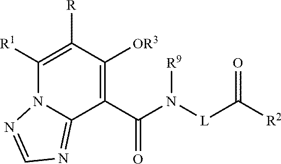

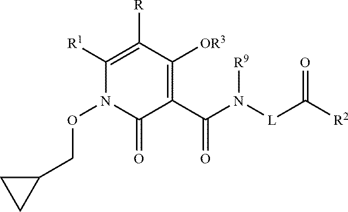

or a pharmaceutically acceptable salt thereof, wherein
R and R¹ are each independently:
(i) hydrogen
(ii) substituted or unsubstituted phenyl;
(iii) substituted or unsubstituted heteroaryl; or
(iv) substituted or unsubstituted alkyl; said substitution selected from:
   (i) C₁-C₄ alkyl;
   (ii) C₃-C₄ cycloalkyl; (iii) C₁-C₄ alkoxy;
   (iV) C₃-C₄ cycloalkoxy;
   (v) C₁-C₄ haloalkyl;
   (vi) C₃-C4 halocycloalkyl;
   (vii) halogen;
   (viii) cyano;
   (ix) NHC(O)R⁴;
   (x) C(O)NR^{5a}R^{5b}_{;}
   (xi) phenyl; and
   (xii) heteroaryl; or
   (xiii) two substituents are taken together to form a fused ring having from 5 to 7 atoms;
R⁴ is a C₁-C₄ alkyl or C₃-C₄ cycloalkyl;
R^{5a} and R^{5b} are each independently selected from:
   (i) hydrogen;
   (ii) C₁-C₄ alkyl;
   (iii) C₃-C₄ cycloalkyl; or
   (iv) R^{5a} and R^{5b} are taken together to form a ring having from 3 to 7 atoms; R² is selected from:
   (i) OR⁶
   (ii) NR^{7a}R^{7b}; and
R⁶ is selected from hydrogen and C₁-C₄ alkyl or C₃-C₄ cycloalkyl;
R^{7a} and R^{7b} are each independently selected from:
   (i) hydrogen;
   (ii) C₁-C₄ alkyl or C₃-C₄ cycloalkyl; or
   (iii) R^{7a} and R^{7b} are taken together to form a ring having from 3 to 7 atoms;
R³ is selected from hydrogen, methyl, and ethyl;
L is a linking unit having a structure -[C(R^{8a}R^{8b})]ₙ-R^{8a} and R^{8b} are each independently selected from hydrogen, methyl and ethyl;
n is an integer from 1 to 3; and
R⁹ is selected from hydrogen and methyl.

149. The method of any one of paragraphs 1-147, wherein the HIF prolyl hydroxylase inhibitor or the HIF-alpha stabilizer has a structure of Formula (IV) 3

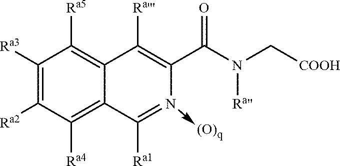

or a pharmaceutically acceptable salt thereof, wherein
q is zero or one;
R^{a1} is selected from the group consisting of hydrogen, alkyl, Substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted hetero cyclic, and R^{a7} is hydrogen, alkyl or aryl or, when X is —NR^{a7}— , then R^{a7} and R^{a6}, together with the nitrogen atom to which they are bound, can be joined to form a heterocyclic or substituted heterocyclic group;
R^{a2} and R^{a3} are independently selected from the group consisting of hydrogen, alkyl, substituted alkyl, aryl, substituted aryl, heteroaryl, substituted heteroaryl, halo, hydroxy, cyano. —S(O)ₙ—N(R^{a6})—R^{a6} where n is 0, 1, or 2, —NR^{a6}C(O)NR^{a6}R^{a6}, —XR^{a6} where X is oxygen, —S(O)ₙ—, or —NR^{a7}— where n is zero, one or two, each R^{a6} is independently selected from the group consisting of hydrogen, alkyl, Substituted alkyl, aryl, substituted aryl, cycloalkyl, substituted cycloalkyl, heteroaryl, substituted heteroaryl, heterocyclic and substituted heterocyclic provided that when when X is —SO— or —SO₂— , then R^{a6} is not hydrogen, and R^{a7} is selected from the group consisting of hydrogen, alkyl, aryl, or R^{a2}, R^{a3} together with the carbon atom pendent thereto, form an aryl substituted aryl, heteroaryl, or substituted heteroaryl;
R^{a4} and R^{a5} are independently selected from the group consisting of hydrogen, halo, alkyl, substituted alkyl, alkoxy, Substituted alkoxy, aryl, substituted aryl, heteroaryl, substituted heteroaryl and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl or, when X is —NR^{a7}—, then R^{a7} and R^{a6}, together with the nitrogen atom to which they are bound, can be joined to form a heterocyclic or Substituted heterocyclic group;
R^{a″} is selected from the group consisting of hydrogen, alkyl and substituted alkyl;
R^{a‴} is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)—R^{a10} wherein R^{a10} is selected from the group consisting of alkyl, substituted alkyl, cycloalkyl, substituted cycloalkyl, aryl, substituted aryl, heteroaryl and substituted heteroaryl and n is zero, one or two;
or a pharmaceutically acceptable salt, ester, or prodrug thereof;
with the proviso that when R^{a″} is hydrogen and q is zero, then at least one of the following occurs:
1) R^{a1} is fluoro, bromo, iodo, alkyl, substituted alkyl, alkoxy, aminoacyl, Substituted alkoxy, aryl, substituted aryl, heteroaryl, substituted heteroaryl, heterocyclic, substituted heterocyclic, and —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl; or
2) R^{a2} is substituted alkyl, aryl, substituted aryl, heteroaryl, substituted heteroaryl, fluoro, bromo, iodo, cyano, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a2} is substituted alkyl such a substituent does not include trifluoromethyl;
   b) —XR^{a6} is not alkoxy; and
   c) when —XR^{a6} is substituted alkoxy such a substituent does not include benzyl or benzyl substituted by a substituent selected from the group consisting of (C₁-C₅) alkyl and (C₁-C₅) alkoxy or does not include a fluoroalkoxy substituent of the formula:
   —O—[CH₂]ₓ—C_{f}H_{(2f+1-g)}F_{g}
where x is zero or one; f is an integer of from 1 to 5; and
g is an integer of from 1 to (2f+1); or
3) R^{a3} is substituted alkyl, aryl, substituted aryl, heteroaryl, substituted heteroaryl, bromo, iodo, —XR^{a6} where X is oxygen—S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a3} is substituted alkyl such a substituent does not include trifluoromethyl;
   b) —XR^{a6} is not alkoxy; and
   c) when XR^{a6} is substituted alkoxy such a substituent does not include benzyl or benzyl substituted by a substituent selected from the group consisting of (C₁-C₅) alkyl and (C-C) alkoxy or does not include a fluoroalkoxy substituent of the formula:
   —O—[CH₂]ₓ—CfH_{(2f+1-g)}F_{g}
   where x is zero or one; f is an integer of from 1 to 5; and
   g is an integer of from 1 to (2f+1); or
4) R^{a4} is iodo, substituted alkyl, aryl, substituted aryl, heteroaryl, substituted heteroaryl, —XR^{a6} where X is oxygen—S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R^{a4} is substituted alkyl such a substituent does not include trifluoromethyl;
   b) —XR^{a6} is not alkoxy; and
   c) when XR^{a6} is substituted alkoxy such a substituent does not include benzyl or benzyl substituted by a substituent selected from the group consisting of (C₁-C₅) alkyl and (C-C) alkoxy or does not include a fluoroalkoxy substituent of the formula:
   —O—[CH₂]ₓ—C_{f}H_{(2f+1-g)}F_{g} where x is zero or one; f is an integer of from 1 to 5; and
   g is an integer of from 1 to (2f+1); or
5) R^{a5} is iodo, substituted alkyl, aryl, substituted aryl, heteroaryl, substituted heteroaryl, —XR^{a6} where X is oxygen, —S(O)ₙ—, or —NR^{a7}— where n is zero, one or two, R^{a6} is selected from the group consisting of alkyl, substituted alkyl, aryl, substituted aryl, heteroaryl, substituted heteroaryl, heterocyclic and substituted heterocyclic, and R^{a7} is hydrogen, alkyl or aryl provided that:
   a) when R²⁵ is substituted alkyl such a substituent does not include trifluoromethyl;
   b) —XR^{a6} is not alkoxy; and
   c) when XR^{a6} is substituted alkoxy such a substituent does not include benzyl or benzyl substituted by a substituent selected from the group consisting of (C₁-C₅) alkyl and (C-C) alkoxy or does not include a fluoroalkoxy substituent of the formula:
   —O—[CH₂]ₓ—C_{f}H_{(2f+1-g)}F_{g} where x is zero or one; f is an integer of from 1 to 5; and
   g is an integer of from 1 to (2f+1);
   and with the further following proviso:
   that when R^{a1}, R^{a3}, R^{a4}, and R^{a5} are hydrogen, then R^{a2} is not bromo.

150. The method of any one of paragraphs 1-147, wherein the HIF prolyl hydroxylase inhibitor or the HIF-alpha stabilizer has a structure of Formula (V)

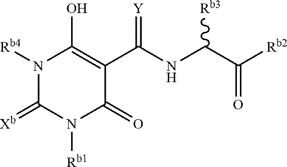

or a pharmaceutically acceptable salt thereof, wherein
R^{b1} and R^{b4} are each independently selected from the group consisting of hydrogen, — NR^{b5}R^{b6}, C₁-C₁₀ alkyl, C₂-C₁₀ alkenyl, C₂-C₁₀ alkynyl, C₃-C₈ cycloalkyl, C₃-C₈ cycloalkyl-C₁-C₁₀ alkyl, C₅-C₈ cycloalkenyl, C₅-C₈ cycloalkenyl- C₁-C₁₀ alkyl, C₃-C₈ heterocycloalkyl, C₃-C₈ heterocycloalkyl—C₁—C₁₀ alkyl, aryl, aryl- C₁-C₁₀ alkyl, heteroaryl and heteroaryl- C₁-C₁₀ alkyl;
R^{b2} is —NR^{b7}R^{b8} or —OR^{b9};
R^{b3} is H or C₁-C₄ alkyl;
where R^{b5} and R^{b6} are each independently selected from the group consisting of hydrogen, C₁-C₁₀ alkyl, C₃-C₈ cycloalkyl, C₃-C₈ cycloalkyl-C₁-C₁₀ alkyl, C₃-C₈ heterocycloalkyl, C₃-C₈ heterocycloalkyl—C₁—C₁₀ alkyl, aryl, aryl- C₁-C₁₀ alkyl, heteroaryl and heteroaryl- C₁-C₁₀ alkyl, —C(O)C₁-C₄ alkyl, —C(O)C₃-C₆ cycloalkyl, —C(O)C₃-C₆ heterocycloalkyl, —C(O)aryl, —C(O)heteroaryl and —S(O)₂ C₁-C₄ alkyl, or, when R^{b5} and R^{b6} are attached to the same nitrogen, R^{b5} and R^{b6} taken together with the nitrogen to which they are attached forma 5- or 6- or 7-membered saturated ring optionally containing one other heteroatom selected from oxygen, nitrogen and sulphur,
R^{b7} and R^{b8} are each independently selected from the group consisting of hydrogen, C₁-C₁₀ alkyl, C₂-C₁₀ alkenyl, C₃-C₈ cycloalkyl, C₃-C₈ heterocycloalkyl, aryl, and heteroaryl, and
R^{b9} is H or a cation, or C₁-C₁₀ alkyl which is unsubstituted or substituted with one or more substituents independently selected from the group consisting of C₃-C₆ cycloalkyl, heterocycloalkyl, aryl and heteroaryl;
X^{b} is O or S; and
Y is O or S;
where any carbon or heteroatom of R^{b1}, R^{b2}, R^{b3}, R^{b4}, R^{b5}, R^{b6}, R^{b7}, R^{b8}, R^{b9} is unsubstituted or is substituted with one or more substituents independently selected from C₁-C₆ alkyl, C₁-C₆ haloalkyl, halogen, —R^{b10}, —NR^{b5}R^{b6}, oxo, cyano, nitro, —C(O)R^{b10}, —C(O)OR^{b10}, —SR^{b10}, —S(O)R^{b10}, —S(O)₂R^{b10}, —CONR^{b5}R^{b6}, —N(R^{b5})C(O)R^{b10}, —N(R^{b5})C(O)OR^{b10},
—OC(O)NR^{b5}R^{b6}', —N(R^{b5})C(O)NR^{b5}R^{b6}, —SO₂NR^{b5}R^{b6}, —N(R^{b5})SO₂R^{b10}, C₂-C₁₀ alkenyl,
C₂-C₁₀ alkynyl, C₃-C₆ cycloalkyl, C₃-C₆ heterocycloalkyl, aryl, C₁-C₆ alkyl-aryl, heteroaryl and C₁-C₆ alkyl-heteroaryl, wherein R^{b5} and R^{b6} are the same as defined above and R^{b10} is selected from hydrogen, C₁-C₁₀ alkyl, C₂-C₁₀ alkenyl, C₂-C₁₀ alkynyl. — C(O)C₁-C₄ alkyl, —C(O)aryl,
—C(O)heteroaryl, —C(O)C₃-C₆ cycloalkyl, —C(O)C₃-C₆ heterocycloalkyl, —S(O)₂C₁-C₄ alkyl, C₃-C₈ cycloalkyl, C₃-C₈ heterocycloalkyl, C₆-C₁₄ aryl, aryl-C₁-C₁₀ alkyl, heteroaryl and heteroaryl-C₁-C₁₀ alkyl.

151. The method of any one of paragraphs 1-147, wherein the HIF prolyl hydroxylase inhibitor or the HIF-alpha stabilizer has a structure of Formula (VI)

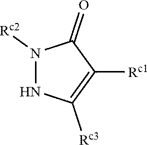

or a pharmaceutically acceptable salt thereof, wherein
R^{c1} represents a heteroaryl group of the formula

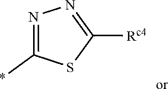

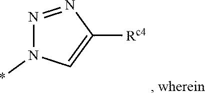

# denotes the linkage point with the dihydropyrazolone ring and
R^{c4} denotes hydrogen, fluorine, chlorine, bromine, cyano, C₁-C₄ alkyl, trifluoromethyl, hydroxymethyl, C₁-C₄ alkoxy, trifluoromethoxy, hydroxycarbonyl or C₁-C₄ alkoxycarbonyl;
R^{c2} represents a heteroaryl group of the formula

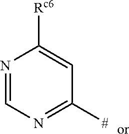

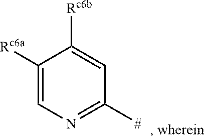

# denotes the linkage point with the dihydropyrazolone ring and
R^{c6}, R^{c6a} and R^{c6b} are identical or different and independently of one another denote hydrogen or a substituent chosen from the group consisting of fluorine, chlorine, bromine, cyano, C₁-C₆ alkyl, trifluoromethyl, hydroxyl, C₁-C₆ alkoxy, trifluoromethoxy, amino, mono-C₁-C₄ alkylamino, di-C₁-C₄ alkylamino, hydroxycarbonyl, C₁-C₄ alkoxycarbonyl, 4-to 6-membered heterocycloalkyl, phenyl and 5-or 6-membered heteroaryl, wherein
C₁-C₆ alkyl in its turn can be substituted by hydroxyl, C₁-C₄ alkoxy or amino and
4-to 6-membered heterocycloalkyl, phenyl and 5-or 6-membered heteroaryl in their turn can in each case be substituted once or twice in an identical or different manner by fluorine, chlorine, bromine, cyano, C₁-C₄ alkyl, trifluoromethyl, hydroxyl, C₁-C₄ alkoxy, trifluoromethoxy, oxo, amino, mono-C₁-C₄ alkylamino, di-C₁-C₄ alkylamino, hydroxycarbonyl or C₁-C₄ alkoxycarbonyl, and
R^{c3} represents hydrogen, or a salt thereof.

152. The method of any one of paragraphs 1-151, wherein the HIF prolyl hydroxylase inhibitor or a HIF-alpha stabilizer, or a pharmaceutically acceptable salt thereof, is administered orally, intravenously, intramuscularly, by inhalation or transdermally.

153. The method of paragraph 152, wherein the HIF prolyl hydroxylase inhibitor or a HIF-alpha stabilizer, or a pharmaceutically acceptable salt thereof, is administered orally.

154. The method of any one of paragraphs 1-153, wherein the HIF prolyl hydroxylase inhibitor or a HIF-alpha stabilizer, or a pharmaceutically acceptable salt thereof, is administered to a subject patient at a dose of about 100-1500, 100-1800, or 150-1800 mg per day.

155. The method of any one of paragraphs 1-151, wherein the HIF prolyl hydroxylase inhibitor or the HIF-alpha stabilizer is vadadustat (AKB-6548), roxadustat (FG-4592), daprodustat (GSK-12788363), molidustat (BAY 85-3934), enarodustat (JTZ-951) or desidustat (ZYAN1).

156. The method of paragraph 155, wherein the HIF prolyl hydroxylase inhibitor or the HIF-alpha stabilizer is vadadustat (AKB-6548).

157. The method of paragraph 156, wherein vadadustat (AKB-6548) is administered at a dose of 150 mg to 1000 mg or of 150 mg to 1800 mg per day.

158. The method of paragraph 157, wherein vadadustat (AKB-6548) is administered at an initial dose of 900 mg per day.

159. The method of paragraph 156 or 157, wherein vadadustat (AKB-6548) is administered at a dose of 900 mg per day throughout the treatment period.

160. The method of paragraph 156 or 157, comprising administration of vadadustat (AKB-6548) a dose of 900-1800 mg per day.

161. The method of paragraph 160, comprising administration of vadadustat (AKB-6548) at a dose of 900, 1050, 1200, 1350, 1500, 1650, or 1800 mg per day.

162. The method of any one of paragraphs 1-161, wherein the HIF prolyl hydroxylase inhibitor or the HIF-alpha stabilizer is administered to the patient for up to about one month.

163. The method of paragraph 162, wherein the HIF prolyl hydroxylase inhibitor or the HIF-alpha stabilizer is administered to the patient for up to about four weeks (about 28 days), three weeks (about 21 days), two weeks (about 14 days), or one week (about 7 days).

164. The method of any one of paragraphs 1-163, wherein the HIF prolyl hydroxylase inhibitor or the HIF-alpha stabilizer is administered in combination with another treatment.

165. The method of paragraph 164, wherein the treatment comprises an antiviral agent or oxygen therapy.

166. The method of paragraph 165, wherein oxygen therapy comprises mechanical ventilation.

167. The method of any one of paragraphs 1-166, wherein the severity of disease is maintained or reduced following commencement of administering the HIF prolyl hydroxylase inhibitor or the HIF-alpha stabilizer to the patient.

168. The method of paragraph 167, wherein the severity of disease is assessed according to the following ordinal scale where increasing numbers denote increased severity:

| Score | Descriptor | Patient State | |
|---|---|---|---|
| 0 | No clinical or virological evidence of infection | Uninfected | Uninfected/ Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | | |

| Score | Descriptor | Patient State | |
|---|---|---|---|
| 6 | Patient is hospitalized and receives oxygen therapy that is non-invasive ventilation or high flow oxygen devices | Infected -Hospitalized (Severe Disease) | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |

169. The method of paragraph 167 or 168, wherein the severity of disease is maintained and the patient’s condition stabilizes following commencement of administering the HIF prolyl hydroxylase inhibitor or the HIF-alpha stabilizer to the patient.

170. The method of paragraph 168, wherein the patient’s score does not change and the patient’s condition stabilizes following commencement of administering the HIF prolyl hydroxylase inhibitor or the HIF-alpha stabilizer to the patient.

171. The method of paragraph 167 or 168, wherein the severity of disease decreases following commencement of administering the HIF prolyl hydroxylase inhibitor or the HIF-alpha stabilizer to the patient.

172. The method of paragraph 168, wherein the patient’s score decreases by at least one point following commencement of administering the HIF prolyl hydroxylase inhibitor or the HIF-alpha stabilizer to the patient.

173. The method of paragraph 168, wherein the patient’s severity of disease is assessed as uninfected/ambulatory.

174. The method of paragraph 168, wherein the patient’s severity of disease is assessed as hospitalized but not intubated.

175. The method of paragraph 168, wherein the patient’s severity of disease is assessed as hospitalized and intubated.

176. The method of paragraph 168, wherein the patient’s category decreases by at least one category following commencement of administering the HIF prolyl hydroxylase inhibitor or the HIF-alpha stabilizer to the patient.

177. The method of any one of paragraphs 167-172, wherein the patient is hypoxic.

178. The method of paragraph 177, wherein the patient has an oxygen saturation value that is about 94% or less.

179. The method of paragraph177 or 178, wherein the patient’s oxygen saturation value improves to a value of about 95% or greater following commencement of administering the HIF prolyl hydroxylase inhibitor or the HIF-alpha stabilizer to the patient.

180. The method of any one of paragraphs 167-179, wherein the patient has hypotension.

181. The method of paragraph 180, wherein the patient receives vasopressor therapy.

182. The method of paragraph 181, wherein vasopressor therapy is reduced or discontinued following commencement of administering the HIF prolyl hydroxylase inhibitor or the HIF-alpha stabilizer to the patient.

183. The method of any one of paragraphs 167-182, wherein the patient is hospitalized.

184. The method of paragraph 183, wherein the patient does not receive oxygen therapy.

185. The method of paragraph 183, wherein the patient has a score of 1, 2, and/or 3 according to the ordinal scale of paragraph 406.

186. The method of paragraph 183, wherein the patient receives oxygen therapy.

187. The method of paragraph 186, wherein the patient has a score of 4, 5, 6, or 7 according to the ordinal scale of paragraph 406.

188. The method of paragraph 186 or 187, wherein the patient receives oxygen therapy by mask or nasal prongs.

189. The method of paragraph 186 or 187, wherein the patient receives oxygen therapy by intubation and mechanical ventilation.

190. The method of paragraph 189, wherein the patient has multi-organ injuries and receives additional organ support therapy.

191. The method of paragraph 189, wherein the patient has any of a cardiovascular injury, a neurological injury, a liver injury, a pancreas injury, and/or a kidney disease.

192. The method of paragraph 191, wherein the patient has a heart injury and/or a lung injury.

193. The method of any one of paragraphs 189-192, wherein the patient receives organ support therapy that is vapopressor therapy, renal replacement therapy (RRT), and/or extracorporeal membrane oxygenation (ECMO).

194. The method of any one of paragraphs 189-193, wherein the organ support therapy received by the patient is discontinued following commencement of administering the HIF prolyl hydroxylase inhibitor or the HIF-alpha stabilizer to the patient.

195. The method of any one of paragraphs 186-194, wherein the oxygen therapy received by the patient is discontinued following commencement of administering the HIF prolyl hydroxylase inhibitor or the HIF-alpha stabilizer to the patient.

196. The method of any one of paragraphs 189-195, wherein the patient has increased ventilator-free survival following discontinuation of mechanical ventilation.

197. The method of any one of paragraphs 167-196, wherein the patient receives vadadustat, or a pharmaceutically acceptable salt thereof.

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

1. A method of treating a respiratory viral infection, comprising administering to a patient in need thereof an oral, once daily dose of vadadustat, or a pharmaceutically acceptable salt thereof.

2. The method of claim 1, wherein the respiratory viral infection is induced by or associated with a virus that could lead to viral pneumonia.

3. The method of claim 1, wherein the respiratory viral infection is induced by or associated with a coronavirus, an influenza A, an influenza B, a Rhinovirus, a respiratory syncytial virus (RSV), an adenovirus, a human parainfluenza virus (HPIV) or an enterovirus.

4. The method of claim 1, wherein the respiratory viral infection is induced by or associated with a coronavirus.

5. The method of claim 4, wherein the coronavirus is the SARS-CoV, MERS-CoV, or SARS-CoV-2.

6-8. (canceled)

9. The method of any claim 1, wherein the respiratory viral infection is COVID-19.

10. The method of claim 9, wherein the respiratory viral infection induces a lung disease.

11. The method of claim 10, wherein the lung disease is selected from acute lung injury (ALI), bronchitis, pneumonia, pulmonary fibrosis, asthma, or acute respiratory distress syndrome (ARDS).

12-19. (canceled)

20. A method of treating or preventing a lung disease that is acute lung injury (ALI) in a patient having a respiratory viral infection, comprising administering to the patient an oral, once daily dose of vadadustat, or a pharmaceutically acceptable salt thereof.

21. A method of treating or preventing a lung disease that is acute respiratory distress syndrome (ARDS) in a patient having a respiratory viral infection, comprising administering to the patient an oral, once daily dose of vadadustat, or a pharmaceutically acceptable salt thereof.

22-23. (canceled)

24. The method of claim 21, wherein the respiratory viral infection is infection by a coronavirus.

25. The method of claim 24, wherein the coronavirus is the SARS-CoV, MERS-CoV, or SARS-CoV-2.

26. The method of claim 20, wherein the respiratory viral infection is COVID-19.

27. The method of claim 20, wherein the patient with acute lung injury develops acute respiratory distress syndrome (ARDS).

28-155. (canceled)

156. The method of claim 1, wherein the patient has a positive diagnostic test for SARS-CoV-2.

157-158. (canceled)

159. The method of claim 1, wherein vadadustat is administered at an initial dose of 900 mg per day.

160. The method of claim 1, wherein vadadustat is administered at a dose of 900 mg per day throughout the treatment period.

161. The method of claim 1, comprising administration of vadadustat a dose of 900-1800 mg per day.

162-164. (canceled)

165. The method of claim 1, wherein vadadustat is administered in combination with another treatment.

166-167. (canceled)

168. The method of claim 1, wherein the severity of disease is maintained or reduced following commencement of administering vadadustat to the patient.

169. The method of claim 168, wherein the severity of disease is assessed according to the following ordinal scale where increasing numbers denote increased severity:

| Score | Descriptor | Patient State | |
|---|---|---|---|
| 0 | No clinical or virological evidence of infection | Uninfected | Uninfected/ Ambulatory |
| 1 | Not hospitalized, no limitation of patient’s activities | Infected (Ambulatory) | |
| 2 | Not hospitalized, limitation of patient’s activities and/or requiring home oxygen | | |
| 3 | Patient is hospitalized but does not receive oxygen therapy - no longer requires ongoing medical care | Infected -Hospitalized (Mild Disease) | Hospitalized but not intubated |
| 4 | Patient is hospitalized but does not require oxygen therapy -requiring ongoing care (COVID-19 related or otherwise) | | |
| 5 | Patient is hospitalized and requiring supplemental oxygen | Infected -Hospitalized (Severe Disease) | |
| 6 | Patient is hospitalized and receives oxygen therapy that is non-invasive ventilation or high flow oxygen devices | | Hospitalized and intubated |
| 7 | Patient is hospitalized and receives invasive mechanical ventilation and additional organ support such as pressors, renal replacement therapy (RRT), and/or extracorporeal membrane oxygenation (ECMO) | | |

.

170-192. (canceled)

193. The method of claim 169, wherein the patient has a heart injury and/or a lung injury.

194-198. (canceled)
